SEQUENCE LISTING (51 sequences)

SEQ ID NO: 1 - DNA, 1275 bp, Mus musculus
  source 1..1275: mol_type = genomic DNA
atgcccttgctttggctgagaggatttctgttggcaagttgctggattatagtgaggagttcccccaccccaggatccgaggggcacggctcagccccggactgcccgtcctgtgcgctggccacccttccgaaggatggacctaactctcagccagagatggtagaggctgtcaagaagcacatcttaaacatgctgcacttgaagaagagacccgatgtcacccagccggtgcccaaggcggcgcttctcaacgcgatcagaaagcttcatgtgggtaaagtgggggagaacgggtatgtggagatagaggacgacattggcaggagggccgaaatgaatgaactcatggagcagacctcggagatcatcacctttgccgagtcaggcacagccaggaagacactgcactttgagatttccaaggaaggcagtgacctgtcagtagtggagcgtgcagaagtgtggctcttcctgaaagtccccaaggctaacagaaccaggaccaaagtcaccatccgtctatttcagcagcagaagcacccacagggcagcttggacacgggggatgaggccgaggaaatgggcttaaagggggagaggagtgaactgttgctatcagagaaagtagttgatgctcggaagagtacctggcacatctttccagtgtccagcagcatccagcgcctgctggaccagggaaagagttccctggacgtgcggattgcttgtgagcagtgccaggagagtggtgccagtctagtgcttctgggcaagaagaagaagaaagaggtggatggagatgggaagaagaaagatgggagtgacggagggctggaagaggaaaaggaacagtcacatagacctttcctcatgctgcaggctaggcagtccgaagaccaccctcatcgcaggcgtaggcggggcttggagtgcgacggcaaggtcaacatttgctgtaagaaacagttctttgtcagcttcaaggacattggctggaatgactggatcattgctccctctggctatcacgccaattattgtgagggggagtgcccaagccacatagcaggcacctctgggtcctcgctctccttccactcaacagtcattaaccactaccgcatgaggggtcacagcccctttgccaaccttaagtcatgctgtgtgcccaccaagctgagacccatgtccatgctgtattacgatgatggtcaaaacatcatcaaaaaggacattcaaaacatgattgtggaggagtgtggctgctcctag

SEQ ID NO: 2 - DNA, 1281 bp, Homo sapiens
  source 1..1281: mol_type = genomic DNA
atgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatag

SEQ ID NO: 3 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgtctttcttcttcttcttgccctgttctggtggtacccagggcaagatcaggaagaaagacacagaggcctgcctggccctcgagagactgccctgactgaaggccctatcaggtgggggaggggatcctgatagagggcactgctgccactgttggggcccaag

SEQ ID NO: 4 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgtttgccactgtcttctctggatgttctggtggtacccatccagagatcatagtggcaaacacagaggcctgcctggccctcgagagactgccctgactgaaggccctatcaggtgggggaggggatcctgatagagggcactgctgccactgttggggcccaag

SEQ ID NO: 5 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgttcctttcaagtatctctccttgttctggtggtacccaaggagagaatcctgaaaggaacacagaggcctgcctggccctcgagagactgccctgactgaaggccctatcaggtgggggaggggatcctgatagagggcactgctgccactgttggggcccaag

SEQ ID NO: 6 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaag

SEQ ID NO: 7 - DNA, 269 bp, synthetic construct
  source 1..269: mol_type = other DNA
ggcagccttggagtgggttcctgccccctcgggcacacaaacagagctgaagaccaccctgggcacctccttggctggccgcatacctcctggcgggcagctgtgttttgatgatgttttgaccattgttctggtggtacccaatggtcaattcgtcatcaaaacacagaggcctgcctggccctcgagagactgccctgactgaaggccctatcaggtgggggaggggatcctgatagagggcactgctgccactgttggggcccaag

SEQ ID NO: 8 - DNA, 269 bp, synthetic construct; identical to SEQ ID NO: 3
  source 1..269: mol_type = other DNA

SEQ ID NO: 9 - DNA, 2371 bp, synthetic construct
  source 1..2371: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggcagccttggagtgggttcctgccccctcgggcacacaaacagagctgaagaccaccctgggcacctccttggctggccgcatacctcctggcgggcagctgtgtctttcttcttcttcttgccctgttctggtggtacccagggcaagatcaggaagaaagacacagaggcctgcctggccctcgagagactgccctgactgaaggccctatcaggtgggggaggggatcctgatagagggcactgctgccactgttggggcccaaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 2371 bp, synthetic construct
  source 1..2371: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggcagccttggagtgggttcctgccccctcgggcacacaaacagagctgaagaccaccctgggcacctccttggctggccgcatacctcctggcgggcagctgtgtttgccactgtcttctctggatgttctggtggtacccatccagagatcatagtggcaaacacagaggcctgcctggccctcgagagactgccctgactgaaggccctatcaggtgggggaggggatcctgatagagggcactgctgccactgttggggcccaaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 2371 bp, synthetic construct
  source 1..2371: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggcagccttggagtgggttcctgccccctcgggcacacaaacagagctgaagaccaccctgggcacctccttggctggccgcatacctcctggcgggcagctgtgttcctttcaagtatctctccttgttctggtggtacccaaggagagaatcctgaaaggaacacagaggcctgcctggccctcgagagactgccctgactgaaggccctatcaggtgggggaggggatcctgatagagggcactgctgccactgttggggcccaaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 2371 bp, synthetic construct
  source 1..2371: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 2371 bp, synthetic construct
  source 1..2371: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggcagccttggagtgggttcctgccccctcgggcacacaaacagagctgaagaccaccctgggcacctccttggctggccgcatacctcctggcgggcagctgtgttttgatgatgttttgaccattgttctggtggtacccaatggtcaattcgtcatcaaaacacagaggcctgcctggccctcgagagactgccctgactgaaggccctatcaggtgggggaggggatcctgatagagggcactgctgccactgttggggcccaaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 2371 bp, synthetic construct; identical to SEQ ID NO: 9
  source 1..2371: mol_type = other DNA

SEQ ID NO: 15 - DNA, 3167 bp, synthetic construct
  source 1..3167: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagcactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgagggcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctagacgggagcaagctttcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcagtcaccgtcgccgccagggctctgcgtttgctccaggtagtccgctgctcccttgggcctgggcccactgacagccctggtgcctctggccggctgcacacctcctggcgggcagctgtgtgtaatctggtgagccactgttgttctggcaatacctgacagtggcagatcagattacacacggaggcctgccctgactgcccacggtgccgtggccaaagaggatctaagggcaccgctgagggcctacctaaccatcgtggggaataaggacagtgtcacccatttaaatggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaagaccggtgaattccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctgaacaaacaccattgtcacactccaacaaacaccattgtcacactccaacaaacaccattgtcacactccatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 3767 bp, synthetic construct
  source 1..3767: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctttcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcaagggctctgcgtttgctccaggtagtccgctgctcccttgggcctgggcccactgacagccctggtgcctctggccggctgcacacctcctggcgggcagctgtgtgtaatctggtgagccactgttgttctggcaatacctgacagtggcagatcagattacacacggaggcctgccctgactgcccacggtgccgtggccaaagaggatctaagggcaccgctgagggcctacctaaccatcgtggggaataaggacagtgtcaccctgcagtcaccgtcgccgccaccggtggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaagtgaattccgccaccatggtcgatggagtgatgatcctgcctgtcctgattatgattgccctgcccagccccagcatggaagatgaaaaacctaaagtcaaccctaagctgtatatgtgcgtgtgcgagggcctgagctgcggaaacgaggatcactgcgagggccagcagtgtttcagctccctgtccatcaatgacggcttccacgtgtaccagaagggctgctttcaggtgtatgagcagggcaagatgacctgtaagacaccaccttccccaggacaggcagtggagtgctgtcagggcgattggtgtaaccggaatatcaccgcccagctgccaacaaagggcaagtctttccccggcacacagaactttcacctggaagtgggcctgatcatcctgagcgtggtgttcgccgtgtgcctgctggcatgtctgctgggagtggccctgagaaagtttaagcggagaaaccaggagcggctgaatccaagagatgtggagtacggcaccatcgagggcctgatcaccacaaatgtgggcgactctacactggccgacctgctggatcacagctgcaccagcggctccggatctggcctgccctttctggtgcagaggaccgtggcccggcagatcaccctgctggagtgcgtgggcaagggccggtacggagaagtgtggagaggatcctggcagggagagaacgtggcagtgaagatcttctctagccgggatgagaagtcttggtttagagagacagagctgtataacacagtgatgctgaggcacgagaatatcctgggcttcatcgcctccgacatgacctctcgccactcctctacacagctgtggctgatcacccactaccacgagatgggctccctgtacgattacctccagctgaccacactggacacagtgtcttgcctgcggatcgtgctgtctatcgccagcggcctggcacacctgcacatcgagatctttggaacccagggcaagccagcaatcgcacacagagatctgaagtctaagaacatcctggtgaagaagaatggccagtgctgtatcgccgatctgggcctggccgtgatgcacagccagtccaccaaccagctggacgtgggcaacaatcctcgggtgggcacaaagagatacatggccccagaggtgctggatgagacaatccaggtggactgcttcgatagctataagagggtggacatctgggcctttggcctggtgctgtgggaggtggcaaggaggatggtgagcaacggcatcgtggaggactacaagccacccttctatgacgtggtgcctaatgatccatcctttgaggacatgcgcaaggtggtgtgcgtggatcagcagaggcccaacatccctaatcgctggttcagcgaccccaccctgacatccctggccaagctgatgaaggagtgttggtatcagaatcctagcgccaggctgaccgccctgcgcatcaagaaaactctgactaaaatcgacaatagcctggataaactgaaaaccgactgctgacctgaacaaacaccattgtcacactccaacaaacaccattgtcacactccaacaaacaccattgtcacactccatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 3240 bp, synthetic construct
  source 1..3240: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagcactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgagggcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctagacgggagcaagctttcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcagtcaccgtcgccgccagggctctgcgtttgctccaggtagtccgctgctcccttgggcctgggcccactgacagccctggtgcctctggccggctgcacacctcctggcgggcagctgtgtgtaatctggtgagccactgttgttctggcaatacctgacagtggcagatcagattacacacggaggcctgccctgactgcccacggtgccgtggccaaagaggatctaagggcaccgctgagggcctacctaaccatcgtggggaataaggacagtgtcacccatttaaatggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaagaccggtgccaccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagtttgatgtacaagtaacctgaacaaacaccattgtcacactccaacaaacaccattgtcacactccaacaaacaccattgtcacactccatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 2988 bp, synthetic construct
  source 1..2988: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagcactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgagggcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctagacgggagcaagctttcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcagtcaccgtcgccgccagggctctgcgtttgctccaggtagtccgctgctcccttgggcctgggcccactgacagccctggtgcctctggccggctgcacacctcctggcgggcagctgtgtgtaatctggtgagccactgttgttctggcaatacctgacagtggcagatcagattacacacggaggcctgccctgactgcccacggtgccgtggccaaagaggatctaagggcaccgctgagggcctacctaaccatcgtggggaataaggacagtgtcacccatttaaatggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaagaccggtgccaccatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtaatgtacaagtaacctgaacaaacaccattgtcacactccaacaaacaccattgtcacactccaacaaacaccattgtcacactccatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 3834 bp, synthetic construct
  source 1..3834: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagcactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgagggcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctagacgggagcaagctttcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcagtcaccgtcgccgccagggctctgcgtttgctccaggtagtccgctgctcccttgggcctgggcccactgacagccctggtgcctctggccggctgcacacctcctggcgggcagctgtgtgtaatctggtgagccactgttgttctggcaatacctgacagtggcagatcagattacacacggaggcctgccctgactgcccacggtgccgtggccaaagaggatctaagggcaccgctgagggcctacctaaccatcgtggggaataaggacagtgtcacccatttaaatggcagccttggagtgggttcctgccccctcgggcacacaaacagagctgaagaccaccctgggcacctccttggctggccgcatacctcctggcgggcagctgtgtgatctccgaggtctgctccatgttctggtggtacccatggagcagtgcccggagatcacacagaggcctgcctggccctcgagagactgccctgactgaaggccctatcaggtgggggaggggatcctgatagagggcactgctgccactgttggggcccaagaccggtgccaccatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagggatccggagagggcagaggaagtctgctaacatgcggtgacgtcgaggagaatcctggacctatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagtttgatgtacaagtaacctgaacaaacaccattgtcacactccaacaaacaccattgtcacactccaacaaacaccattgtcacactccatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 4201 bp, synthetic construct
  source 1..4201: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagcactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgagggcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctagacgggagcaagctttcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgtttgctgcccgtgaccagcacgtcaacgattttgtgggcacgggcgacaccgcagtgtagtctgagcagtactcgttgctgccgcgcgcgccaccagacataatagctgacagactaacagactgttcctttccatgggtcttttctgcagtcaccgtcgccgccaccggtgaattccgccaccatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagggaagcggagagggcagaggaagtctgctaacatgcggtgacgtcgaggagaatcctggacctatggtcgatggagtgatgatcctgcctgtcctgattatgattgccctgcccagccccagcatggaagatgaaaaacctaaagtcaaccctaagctgtatatgtgcgtgtgcgagggcctgagctgcggaaacgaggatcactgcgagggccagcagtgtttcagctccctgtccatcaatgacggcttccacgtgtaccagaagggctgctttcaggtgtatgagcagggcaagatgacctgtaagacaccaccttccccaggacaggcagtggagtgctgtcagggcgattggtgtaaccggaatatcaccgcccagctgccaacaaagggcaagtctttccccggcacacagaactttcacctggaagtgggcctgatcatcctgagcgtggtgttcgccgtgtgcctgctggcatgtctgctgggagtggccctgggtggaggcgggtctggaggcgggggtagtggcgggggtggaagcatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggcgtacaggcatcacaagatggcctaccctcctgtacttgttccaactcaagacccaggaccacccccaccttctccattactaggtttgaaaccactgcagttattagggggtggaggcgggtcgggtggcggaggtagcggaggcggtggaatgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctgtaatgtacaagtaacctgaacaaacaccattgtcacactccaacaaacaccattgtcacactccaacaaacaccattgtcacactccatgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 756 bp, synthetic construct
  source 1..756: mol_type = other DNA
tacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcacgcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctaga

SEQ ID NO: 22 - DNA, 2091 bp, synthetic construct
  source 1..2091: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgccgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcacgcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaaggtgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctagagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatctagcatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactagctcgacgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaaggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 3767 bp, synthetic construct; identical to SEQ ID NO: 16
  source 1..3767: mol_type = other DNA

SEQ ID NO: 24 - DNA, 135 bp, synthetic construct
  source 1..135: mol_type = other DNA
acaagctttttgctcgtcttatacaagctttttgctcgtcttatacaagctttttgctcgtcttatacaaacaccattgtcacactccaacaaacaccattgtcacactccaacaaacaccattgtcacactcca

SEQ ID NO: 25 - protein, 509 aa, Homo sapiens
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 26 - protein, 509 aa, synthetic construct
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVAHQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 27 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 28 - DNA, 1530 bp, synthetic construct
  source 1..1530: mol_type = other DNA
atggtcgatggagtgatgatcctgcctgtcctgattatgattgccctgcccagccccagcatggaagatgaaaaacctaaagtcaaccctaagctgtatatgtgcgtgtgcgagggcctgagctgcggaaacgaggatcactgcgagggccagcagtgtttcagctccctgtccatcaatgacggcttccacgtgtaccagaagggctgctttcaggtgtatgagcagggcaagatgacctgtaagacaccaccttccccaggacaggcagtggagtgctgtcagggcgattggtgtaaccggaatatcaccgcccagctgccaacaaagggcaagtctttccccggcacacagaactttcacctggaagtgggcctgatcatcctgagcgtggtgttcgccgtgtgcctgctggcatgtctgctgggagtggccctgagaaagtttaagcggagaaaccaggagcggctgaatccaagagatgtggagtacggcaccatcgagggcctgatcaccacaaatgtgggcgactctacactggccgacctgctggatcacagctgcaccagcggctccggatctggcctgccctttctggtgcagaggaccgtggcccggcagatcaccctgctggagtgcgtgggcaagggccggtacggagaagtgtggagaggatcctggcagggagagaacgtggcagtgaagatcttctctagccgggatgagaagtcttggtttagagagacagagctgtataacacagtgatgctgaggcacgagaatatcctgggcttcatcgcctccgacatgacctctcgccactcctctacacagctgtggctgatcacccactaccacgagatgggctccctgtacgattacctccagctgaccacactggacacagtgtcttgcctgcggatcgtgctgtctatcgccagcggcctggcacacctgcacatcgagatctttggaacccagggcaagccagcaatcgcacacagagatctgaagtctaagaacatcctggtgaagaagaatggccagtgctgtatcgccgatctgggcctggccgtgatgcacagccagtccaccaaccagctggacgtgggcaacaatcctcgggtgggcacaaagagatacatggccccagaggtgctggatgagacaatccaggtggactgcttcgatagctataagagggtggacatctgggcctttggcctggtgctgtgggaggtggcaaggaggatggtgagcaacggcatcgtggaggactacaagccacccttctatgacgtggtgcctaatgatccatcctttgaggacatgcgcaaggtggtgtgcgtggatcagcagaggcccaacatccctaatcgctggttcagcgaccccaccctgacatccctggccaagctgatgaaggagtgttggtatcagaatcctagcgccaggctgaccgccctgcgcatcaagaaaactctgactaaaatcgacaatagcctggataaactgaaaaccgactgctga

SEQ ID NO: 29 - DNA, 3332 bp, synthetic construct
  source 1..3332: mol_type = other DNA
gctctttgcagccgccgccgccgccgcagcctccccctcggcgcagggggaacttttctactctctgtgacggtctcccccgccgcccccgggggcaagcccagctggtccgcccgccccgcccgcggtcgtgctcccagcccgagcctctgcgcctcgggaagtttattctgcccgccctcctccccttcctcctcctcttcaccccgcctccccgccctcccccggctctttcccccgggtggctgccgctgccgccgctgccgctgccagcctgctcctcggccagccgcgccggccccagggccgggaagctcccggccccacgcaccgctccgctgcagccaccgcagccgccgccgctctgggccggccctcggcccccggctcccggccccgcagagttccgggctccctcgccggctgcaccgccccgccccgccccgcgccgccccgcgccgcgccgacctgcagcgcccggctgcctcgcactccgcctcccccggctcagcccccggccgcggcgggacccgagcctggagcattggtaagcgtcacactgccaaagtgagagctgctggagaactcataatcccaggaacgcctcttctactcttcgagtaccccagtgaccagagtgagagaagctctgaacgagggcacgcggcttgaaggactgtgggcagatgtgaccaagagcctgcattaagttgtacaatggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttgacattttcatagtgtcaagaaggaagatttgacgttgttgtcattgtccagctgggacctaatgctggcctgactggttgtcagaatggaatccatctgtctccctccccaaatggctgctttgacaaggcagacgtcgtacccagccatgtgttggggagacatcaaaaccaccctaacctcgctcgatgactgtgaactgggcatttcacgaactgttcacactgcagagactaatgttggacagacactgttgcaaaggtagggactggaggaacacagagaaatcctaaaagagatctgggcattaagtcagtggctttgcatagctttcacaagtctcctagacactccccacgggaaactcaaggaggtggtgaatttttaatcagcaatattgcctgtgcttctcttctttattgcactaggaattctttgcattccttacttgcactgttactcttaattttaaagacccaacttgccaaaatgttggctgcgtactccactggtctgtctttggataataggaattcaatttggcaaaacaaaatgtaatgtcagactttgctgcattttacacatgtgctgatgtttacaatgatgccgaacattaggaattgtttatacacaactttgcaaattatttattacttgtgcacttagtagtttttacaaaactgctttgtgcatatgttaaagcttatttttatgtggtcttatgattttattacagaaatgtttttaacactatactctaaaatggacattttcttttattatcagttaaaatcacattttaagtgcttcacatttgtatgtgtgtagactgtaactttttttcagttcatatgcagaacgtatttagccattacccacgtgacaccaccgaatatattactgatttagaagcaaagatttcagtagaattttagtcctgaacgctacggggaaaatgcattttcttcagaattatccattacgtgcatttaaactctgccagaaaaaaataactattttgttttaatctactttttgtatttagtagttatttgtataaattaaataaactgttttcaagtcaaa

SEQ ID NO: 30 - DNA, 260 bp, Gallus gallus
  source 1..260: mol_type = genomic DNA
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 31 - DNA, 382 bp, unidentified
  source 1..382: mol_type = genomic DNA; note = cytomegalovirus
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 32 - DNA, 23 bp, synthetic construct
  source 1..23: mol_type = other DNA
acaaacaccattgtcacactcca

SEQ ID NO: 33 - DNA, 22 bp, synthetic construct
  source 1..22: mol_type = other DNA
acaagctttttgctcgtcttat

SEQ ID NO: 34 - DNA, 247 bp, synthetic construct
  source 1..247: mol_type = other DNA
tgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctaga

SEQ ID NO: 35 - DNA, 269 bp, synthetic construct
  source 1..269: mol_type = other DNA
agggctctgcgtttgctccaggtagtccgctgctcccttgggcctgggcccactgacagccctggtgcctctggccggctgcacacctcctggcgggcagctgtgtgtaatctggtgagccactgttgttctggcaatacctgacagtggcagatcagattacacacggaggcctgccctgactgcccacggtgccgtggccaaagaggatctaagggcaccgctgagggcctacctaaccatcgtggggaataaggacagtgtcaccc

SEQ ID NO: 36 - protein, 262 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPV

SEQ ID NO: 37 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 38 - DNA, 220 bp, synthetic construct
  source 1..220: mol_type = other DNA
gcttcgcgccctaagtctgcaggtgacgggctcaggggcgggggctgggtgggggggagcggagaatgctccagcccagtttgccgtctccatggcgaccgcccgcgcggcgccagcctgacagcccgtccgggttttatgaatgggtgacgtcacgggcctggcgtctaacggtctgagccgcttgttcagacgctgacacagaccagcccgggaaagg

SEQ ID NO: 39 - DNA, 528 bp, synthetic construct
  source 1..528: mol_type = other DNA
gctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggcgtacaggcatcacaagatggcctaccctcctgtacttgttccaactcaagacccaggaccacccccaccttctccattactaggtttgaaaccactgcagttatta

SEQ ID NO: 40 - DNA, 525 bp, synthetic construct
  source 1..525: mol_type = other DNA
tctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctg

SEQ ID NO: 41 - DNA, 21 bp, synthetic construct
  source 1..21: mol_type = other DNA
tctttcttcttcttcttgccc

SEQ ID NO: 42 - DNA, 21 bp, synthetic construct
  source 1..21: mol_type = other DNA
tttgccactgtcttctctgga

SEQ ID NO: 43 - DNA, 21 bp, synthetic construct
  source 1..21: mol_type = other DNA
ttcctttcaagtatctctcct

SEQ ID NO: 44 - DNA, 21 bp, synthetic construct
  source 1..21: mol_type = other DNA
tgatctccgaggtctgctcca

SEQ ID NO: 45 - DNA, 21 bp, synthetic construct
  source 1..21: mol_type = other DNA
ttttgatgatgttttgaccat

SEQ ID NO: 46 - DNA, 21 bp, synthetic construct
  source 1..21: mol_type = other DNA
tgtaatctggtgagccactgt

SEQ ID NO: 47 - DNA, 47 bp, synthetic construct
  source 1..47: mol_type = other DNA
nnnggtacaaagaacagtggctcaccagattacactgttggagtnnn

SEQ ID NO: 48 - protein, 9 aa, synthetic construct
RTVAHQITL

SEQ ID NO: 49 - DNA, 47 bp, synthetic construct
  source 1..47: mol_type = other DNA
nnnggtacaaagaacagtggctcgccagattacactgttggagtnnn

SEQ ID NO: 50 - protein, 9 aa, synthetic construct
RTVARQITL

SEQ ID NO: 51 - DNA, 23 bp, synthetic construct
  source 1..23: mol_type = other DNA
ggctcaccagattacactgttgg

(19) United States
(12) Patent Application Publication
(10) Pub. No.: US 2025/0327080 A1
(43) Pub. Date: Oct. 23, 2025

(54) RNAS TARGETING ACTIVIN A SUBUNITS

(71) Applicant: University of Massachusetts, Westborough, MA (US)

(72) Inventors: Jae-Hyuck Shim, Worcester, MA (US); Julia Alterman, Worcester, MA (US); Yeon-Suk Yang, Worcester, MA (US); Nozomi Yamada, Worcester, MA (US); Katherine Gross, Worcester, MA (US); Jillian Caiazzi, Worcester, MA (US)

(73) Assignee: University of Massachusetts, Westborough, MA (US)

(21) Appl. No.: 19/248,996

(22) Filed: Jun. 25, 2025

Related U.S. Application Data
(63) Continuation of application No. PCT/US2024/025340, filed on Apr. 19, 2024.
(60) Provisional application No. 63/497,269, filed on Apr. 20, 2023.
(60) Provisional application No. 63/595,460, filed on Nov. 2, 2023.

(51) Int. Cl. C12N 15/113; A61K 9/00; A61P 19/00; C12N 15/86

(52) U.S. Cl. CPC C12N 15/1138; A61K 9/0019; A61P 19/00; C12N 15/1136; C12N 15/86; C12N 2310/141; C12N 2320/31; C12N 2750/14143; C12N 2750/14145

(57) ABSTRACT

Aspects of the disclosure relate to compositions and methods for treating fibrodysplasia ossificans progressiva (FOP) in a subject. In some aspects, the disclosure provides isolated nucleic acids, and vectors such as rAAV vectors, configured to express transgenes that inhibit (e.g., decrease) expression of an INHBA and/or inhibit (e.g., decrease) expression of an mutated ACVR1 gene and/or promote (e.g., increase) expression of wild-type ACVR1 protein in muscle cells, bone cells or connective tissues.

### RELATED APPLICATIONS

This application claims the benefit under 35 U.S.C. § 119 (c) of the filing date of U.S. Provisional Application No. 63/497,269, filed on Apr. 20, 2023, and U.S. Provisional Application No. 63/595,460, filed on Nov. 2, 2023, the entire content of each of which is incorporated herein by reference.

### REFERENCE TO AN ELECTRONIC SEQUENCE LISTING

The content of the electronic sequence listing (U012070182WO00-SEQ-MSB.xml; Size: 100,866 bytes; and Date of Creation: Apr. 15, 2024) is herein incorporated by reference in its entirety.

### BACKGROUND

Activin A is a known ligand for the Activin A Receptor, type I (ACVR1). Overexpression of Activin A has been associated with shortened survival in ovarian, colon, gastric, breast, and non-small cell lung cancers, as well as neuroblastoma. Likewise, functional experiments have shown that Activin A promotes ovarian tumor proliferation and invasion. Experimental data also support a role for Activin A signaling in mediating cancer stem cell survival. Moreover, in animal models of breast cancer bone metastasis and of multiple myeloma with osteolytic lesions, tumor-secreted Activin A acts as a stimulator of bone degradation, inhibiting osteoblast differentiation and stimulating osteoclast differentiation.

### SUMMARY

The inventors of the disclosure have identified that gene therapy strategies that utilize a combination of genetic knockdown of Activin A and a mutant form of its receptor, ACVR1^{R206H}, (e.g., using artificial miRNAs) with a genetic introduction of a human wild-type ACVR1 (e.g., codon-optimized wild-type ACVR1) provide a successful strategy for suppressing heterotopic ossification (HO) and treating Fibrodysplasia ossificans progressiva (FOP).

Accordingly, aspects of the disclosure relate to compositions and methods for treating fibrodysplasia ossificans progressiva (FOP) in a subject. In some aspects, the disclosure provides isolated nucleic acids, and vectors such as rAAV vectors, configured to express transgenes that inhibit (e.g., decrease) expression of mutated ACVR1 gene and/or INIIBA gene in muscle cells or connective tissues. A transgene generally refers to a nucleic acid encoding one or more gene products (e.g., one or more peptides, proteins, functional RNAs such as miRNAs, or a combination thereof).

Accordingly, in some aspects, the disclosure provides an isolated nucleic acid comprising a transgene comprising a nucleic acid sequence encoding one or more artificial microRNAs (amiR) targeting an INHBA RNA transcript (e.g., as set forth in SEQ ID NOs: 1 or 2), flanked by adeno-associated virus (AAV) inverted terminal repeats (ITRs). In some embodiments, one or more amiRs targeting an INHBA RNA transcript comprises the sequence set forth in any one of SEQ ID NOs: 3-8. In some embodiments, a transgene further comprises one or more amiRs targeting an ACVR1 gene. In some embodiments, one or more amiRs target an ACVR1^{R206H} allele.

In some embodiments, a transgene further comprises a promoter operably linked to a nucleic acid sequence encoding one or more amiRs. In some embodiments, a promoter is a chicken beta actin (CBA) promoter or a flare-up-responsive promoter. In some embodiments, a flare-up-responsive promoter comprises a first portion comprising a NF-κB promoter, and a second portion comprising a bone morphogenic protein (BMP) signaling-responsive promoter (pBRE).

In some embodiments, a transgene further comprises a nucleic acid sequence encoding an ACVR1 protein, a nucleic acid sequence encoding a soluble TNFR2 (sTNFR2) protein, a nucleic acid sequence encoding a soluble IL-1Rα (sIL-1Rα) protein, or a combination thereof. In some embodiments, a nucleic acid sequence encoding an ACVR1 protein, a soluble TNFR2, or a soluble IL-1Rα is codon-optimized.

In some embodiments, a transgene further comprises ene or more miRNA binding sites.

In some aspects, the disclosure provides an isolated nucleic acid comprising a transgene flanked by adeno-associated virus (AAV) inverted terminal repeats (ITRs), wherein the transgene comprises a flare-up-responsive promoter comprising a first portion comprising a NF-κB promoter, and a second portion comprising a bone morphogenic protein (BMP) signaling-responsive promoter (pBRE); a nucleic acid sequence encoding one or more artificial microRNAs (amiR) targeting an INHBA RNA transcript; a nucleic acid sequence encoding one or more artificial microRNAs (amiR) targeting an ACVR1^{R206H} RNA transcript; a nucleic acid sequence encoding a soluble IL-1Rα (sIL-1 Rα) protein; a codon-optimized nucleic acid sequence encoding a wild-type ACVR1 protein; a nucleic acid sequence encoding an Activin A trap comprising an ACVR2A kinase deletion mutant protein and an ACVR2B kinase deletion mutant protein; and one or more miRNA binding sites.

In some embodiments, one or more miRNA binding sites comprise one or more miR-122 binding sites, one or more miR-208a binding sites, or a combination thereof.

In some embodiments, AAV ITRs are AAV2 ITRs.

In some aspects, the disclosure provides a vector comprising an isolated nucleic acid as described herein. In some embodiments, the vector is a plasmid.

In some aspects, the disclosure provides a recombinant adeno-associated virus (rAAV) comprising an isolated nucleic acid as described herein and one or more AAV capsid proteins.

In some embodiments, a capsid protein has a tropism for muscle or bone. In some embodiments, an AAV capsid protein is of a serotype selected from AAV1, AAV2, AAV3, AAV4, AAV5, AAV6, AAV7, AAV8, AAV9, AAV10, or a variant thereof.

In some aspects, the disclosure provides an engineered T cell comprising a chimeric antigen receptor (CAR-T), wherein the T cell has been obtained from a subject having an R206H mutation in an ACVR1 gene, and the cell has been engineered by base-editing to revert the R206H mutation in the ACVR1 gene. In some embodiments, the CAR comprises an antigen binding domain that binds to one or more AAV capsid antigens.

In some aspects, the disclosure provides a method for treating a disease or disorder associated with bone in a subject in need thereof, the method comprising administering an isolated nucleic acid, rAAV, and/or engineered T cell as described herein to the subject.

In some embodiments, a subject is a human.

In some embodiments, administration comprises administration to the muscle of a subject, bone of a subject, or systemic administration to a subject.

In some aspects, the disclosure provides an isolated nucleic acid comprising the sequence set forth in any one of SEQ ID NOs: 3-8, 21, and 24. In some embodiments, an isolated nucleic acid further comprises a miR-33 backbone.

In some aspects, the disclosure provides an rAAV vector comprising the sequence set forth in any one of SEQ ID NOs: 9-20, 22, and 23, with the proviso that the rAAV vector does not comprise an EGFP or luciferase protein coding sequence. In some aspects, the disclosure provides an rAAV comprising the rAAV vector and one or more AAV capsid proteins.

In some aspects, the disclosure provides a composition comprising: (a) a first nucleic acid sequence encoding an inhibitory nucleic acid that targets an INHBA transcript; and (b) a second nucleic acid sequence encoding a wild-type Activin A Receptor, type 1 (ACVR1) protein.

In some aspects, the disclosure provides a composition comprising: (a) a first nucleic acid sequence encoding an inhibitory nucleic acid that targets a mutant ACVR1 transcript; and (b) a second nucleic acid sequence encoding a wild-type Activin A Receptor, type 1 (ACVR1) protein.

In some aspects, the disclosure provides a composition comprising: (a) a first nucleic acid sequence encoding an inhibitory nucleic acid that targets an INHBA transcript; (b) a second nucleic acid sequence encoding a wild-type Activin A Receptor, type 1 (ACVR1) protein; and (c) a third nucleic acid sequence encoding an inhibitory nucleic acid that targets a mutant ACVR1 transcript.

In some embodiments, a mutant ACVR1 transcript is an ACVR1^{R206H}/transcript.

In some embodiments, an inhibitory nucleic acid that targets an INHBA transcript is a double-stranded RNA (dsRNA), small interfering RNA (siRNA), short hairpin RNA (shRNA), microRNA (miRNA), or artificial microRNA (amiR) that targets an INHBA transcript. In some embodiments, an amiR that targets an INHBA transcript comprises a nucleic acid sequence having at least 90%, at least 95%, at least 97.5%, at least 99%, or 100% identity to any one of SEQ ID NOs: 3-8. In some embodiments, an amiR that targets an INHBA transcript comprises a nucleic acid sequence having at least 70%, at least 80% at least 90%, at least 95%, at least 97.5%, or at least 99% identity to SEQ ID NO: 6.

In some embodiments, an inhibitory nucleic acid that targets a mutant ACVR1 transcript is a dsRNA, siRNA, shRNA, miRNA, or artificial microRNA (amiR) that targets a mutant ACVR1 transcript. In some embodiments, an amiR that targets a mutant ACVR1 transcript comprises the sequence set forth in SEQ ID NO: 35.

In some embodiments, an isolated nucleic acid further comprises a promoter operably linked to a first nucleic acid sequence, a second nucleic acid sequence, and/or a third nucleic acid sequence. In some embodiments, a promoter is a chicken beta actin (CBA) promoter. In some embodiments, a promoter is a flare-up-responsive promoter. In some embodiments, a flare-up-responsive promoter comprises a first portion comprising a NF-κB promoter, and a second portion comprising a bone morphogenic protein (BMP) signaling-responsive promoter (pBRE).

In some embodiments, a nucleic acid sequence encoding an ACVR1 protein is codon-optimized. In some embodiments, a nucleic acid sequence encoding the ACVR1 protein comprises a nucleic acid sequence having at least 90%, at least 95%, at least 97.5%, or at least 99% identity to SEQ ID NO: 30.

In some embodiments, a first nucleic acid sequence, a second nucleic acid sequence, and/or a third nucleic acid sequence further comprises one or more miRNA binding sites, optionally wherein the one or more miRNA binding sites are de-targeting miRNA binding sites. In some embodiments, or more miRNA binding sites comprise one or more miR-122 binding sites, one or more miR-208a binding sites, or a combination thereof. In some embodiments, one or more miR-122 binding sites comprise or consist of the nucleic acid sequence set forth in SEQ ID NO: 32. In some embodiments, one or more miR-208a binding sites comprise or consist of the nucleic acid sequence set forth in SEQ ID NO: 33. In some embodiments, the one or more miRNA binding sites comprise or consists of the nucleic acid sequence set forth in SEQ ID NO: 24.

In some embodiments, an ACVR1 protein comprises an amino acid sequence having at least 90%, at least 95%, at least 97.5%, at least 99%, or 100% identity to SEQ ID NO: 25.

In some embodiments, a first nucleic acid sequence, a second nucleic acid sequence, and/or a third nucleic acid sequence are encoded within a single nucleic acid.

In some embodiments, single nucleic acid further comprises one or more adeno-associated virus (AAV) inverted terminal repeats (ITRs).

In some aspects, the disclosure provides a vector comprising the composition of any of the embodiments described herein.

In some aspects, the disclosure provides a recombinant adeno-associated (rAAV) comprising (a) a composition of any of the aspects or embodiments described herein; and (b) an AAV capsid protein. In some embodiments, a rAAV targets skeletal tissue or bone. In some embodiments, a capsid protein is a AAV1, AAV6, AAV7, AAV8, AAV9, AAV-DJ/8, AAV-Rh10, AAV-retro, AAV-PHP.B, AAV8-PHP.eB, or AAV-PHP.S capsid protein.

In some aspects, the disclosure provides a pharmaceutical composition comprising (i) a composition of any of the aspects or embodiments described herein, a vector as described herein, a rAAV as described herein, and (ii) a pharmaceutically acceptable excipient. In some embodiments, a composition is formulated for injection, optionally wherein the injection is transdermal (t.d.) injection.

In some aspects, the disclosure provides a method for preventing or reducing heterotopic ossification (HO) in a subject comprising administering to the subject an effective amount of the isolated nucleic acid, vector, rAAV, engineered T cell, composition, or pharmaceutical composition of any embodiment described herein.

In some aspects, the disclosure provides a method for treating fibrodysplasia ossificans progressiva (FOP) in a subject comprising administering to the subject an effective amount of the isolated nucleic acid, vector, rAAV, engineered T cell, composition, or pharmaceutical composition of any embodiment described herein.

In some aspects, the disclosure provides a method for reducing BMP-Smad1/5 signaling in a subject comprising administering to the subject an effective amount of the isolated nucleic acid, vector, rAAV, engineered T cell, composition, or pharmaceutical composition of any embodiment described herein.

In some embodiments, a subject is a human, optionally wherein the subject has at least one copy of a ACVR1^{R206H} allele.

### BRIEF DESCRIPTION OF DRAWINGS

FIG. 1 shows representative data for development of INHBA-targeting artificial microRNAs (amiR-INHBA), and a combination rAAV vector comprising amiRs targeting INHBA and ACVR1^{R206H}.

FIG. 2 shows representative data indicating inclusion af miR-122 and miR208a binding sites into rAAV vectors de-targets the rAAVs from liver and cardiac tissue in vivo.

FIG. 3 shows a schematic depicting flare-up-responsive promoter comprising an inflammation-responsive promoter (pNF-κB) and a bone morphogenic protein (BMP) signaling-responsive promoter (pBRE).

FIG. 4 shows representative data showing injury-induced activity of flare-up-responsive promoters relative to chicken beta-actin promoter (pCBA) in vivo.

FIG. 5 shows a schematic depicting incorporation of flare-up-responsive promoters into rAAV vectors encoding anti-inflammatory gene products (e.g., soluble TNFR2 (sTNFR2) and soluble IL-1Rα (sIL-1Rα), alone or in combination with amiRs targeting ACVR1^{R206H} and INHBA)

FIG. 6 shows a schematic depicting an rAAV vector comprising a flare-up-responsive promoter, an amiR targeting ACVR^{206H}, an IL-1β inhibitor (sIL-1Rα), and an Activin A trap comprising a codon-optimized ACVR1, ACVR2A (kinase del), and ACVR2B (kinase del). The vector also comprises a miR-122 binding site.

FIG. 7 shows a schematic depicting an immunotherapy protocol in which T-cells are isolated from a patient having fibrodysplasia ossificans progressiva (FOP) characterized by one or more mutations in the ACVR1 gene.

FIG. 8 shows a schematic depicting a procedure for production of CAR-Treg cells that have been gene-edited to correct an ACVR1^{R206H} mutation. From top to bottom, SEQ ID NOs: 47-51 are shown.

FIGS. 9A-9E show that levels of Activin A are elevated in heterotopic ossification (HO) tissues of FOP mice. FIG. 9A shows the assessment of trauma-induced HO of 8-week-old ACVR1^{R206H} mice four weeks after pinch injury was introduced into the right-side gastrocnemius muscle. Trauma-induced HO was assessed by radiography (left) and by measuring protein levels of Activin A in tissue lysates via an enzyme-linked immunosorbent assay (ELISA) (right, n=3). HB: heterotopic bone. FIG. 9B shows protein levels of Activin A as measured by ELISA in the supernatant of bone marrow-derived stromal cells (BMSCs) from ACVR1^{+/+};Prx1 (WT) and ACVR1^{R206H};Prx1 (FOP) mice (n=4). FIGS. 9C-9D show the expression of Activin A as measured by ELISA in the supernatant of mouse (n=4; FIG. 9C) and human (n=3; FIG. 9D) BMSCs cultured under growth (ND) or osteogenic (OBD) conditions in the presence of phosphate-buffered saline (PBS), lipopolysaccharide (LPS), tumor necrosis factor (TNF), or interleukin 1β (IL-1β) for 24 hours. FIG. 9E shows Activin A expression as measured by ELISA in the supernatant of BMSCs from ACVR1^{R206H};Prx1 mice (n=3) transduced by AAV9 carrying a control gene (control) or a gene encoding an inhibitory nucleic acid that targets a mutant ACVR1 transcript and a wild-type ACVR1 protein (amiR-ACVR1^{R206H}, ACVR1^{Opt}). The BMSCs were cultured under osteogenic conditions in the presence of PBS, LPS, TNF, or IL-1β for 24 hours.

FIGS. 10A-10C show the generation of an exemplary nucleic acid comprising a first nucleic acid sequence encoding an inhibitory nucleic acid that targets an INHBA transcript; a second nucleic acid sequence encoding a wild-type ACVR1 protein; and a third nucleic acid sequence encoding an inhibitory nucleic acid that targets a mutant ACVR1 transcript (“AAV9.ACVR1/INHBA.MIR”) for FOP therapy. FIG. 10A depicts a schematic showing AAV vector genome and capsid, wherein the AAV vector genome comprises a chicken β-actin (CBA) promoter, an amiR-ACVR1^{R206H}, an amiR-INHBA, a codon-optimized human ACVR1 (ACVR1^{Opt}), and miR-122 target sequences (TS) packaged into an AAV9 capsid. PA: poly-A sequence(s). FIG. 10B shows reverse transcriptase-PCR (RT-PCR) quantitation of INHBA expression from mouse ACVR1^{R206H};Prx1 BMSCs (left) or human BMSCs (right) transduced by AAV9 carrying an artificial miRNA targeting a control sequence (amiR-ctrl) or an artificial miRNA targeting a INHBA sequence (amiR-INHBA #4) (n=4). FIG. 10C shows the expression of Activin A as measured by ELISA in the supernatant of mouse ACVR1^{R206H};Prx1 BMSCs (left) or human BMSCs (right) transduced with AAV9 carrying amiR-ctrl or amiR-INHBA #4 (n=3). BMSCs were cultured in the presence of PBS, LPS, TNF, IL-1β or PMA for 24 hours. *, P<0.05; **, P<0.01; ***, P<0.001; and ****, P<0.0001 by an unpaired two-tailed Student's t-test (FIG. 10B) and one-way ANOVA test (FIG. 10C).

FIGS. 11A-11F show that delivery of an exemplary nucleic acid comprising a first nucleic acid sequence encoding an inhibitory nucleic acid that targets an INHBA transcript; a second nucleic acid sequence encoding a wild-type ACVR1 protein; and a third nucleic acid sequence encoding an inhibitory nucleic acid that targets a mutant ACVR1 transcript suppresses Activin A signaling and osteogenic differentiation of FOP skeletal progenitors. Mouse ACVR1^{R206H};Prx1 BMSCs were transduced by AAV9 carrying control (ctrl) or amiR-ACVR1^{R206H}.amiR-INHBA.ACVR1^{Opt}.MIR (ACVR1/INHBA) and cultured under osteogenic conditions for 6 days. FIG. 11A shows RT-PCR quantitation of ACVR1^{R206H}, ACVR1^{Opt}; and INHBA expression in said BMSCs; gene expression normalized to Gapdh (n=4). FIG. 11B shows osteogenic differentiation as assessed by ALP activity in said BMSCs (n=5). FIG. 11C shows RT-PCR quantitation of osteogenic genes (Tnalp, Osx, Ibsp, and Bglap) in said BMSCs; gene expression normalized to Gapdh (n=4). Mouse ACVR1^{R206H};Prx1 BMSCs were transduced by ctrl or ACVR1/INHBA AAVs and cultured under osteogenic conditions in the presence of PBS or Activin A (50 ng/ml). FIG. 11D shows the quantitation of ALP activity after 3 days of culture (top) and alizarin red staining after 10 days of culture (bottom) of said BMSCs to assess early and late osteogenic differentiation, respectively (n=5). FIG. 11E shows RT-PCR quantitation of Id1 (left) and Msx2 (right) expression in ctrl and ACVR1/INHBA transduced mouse BMSCs treated with PBS or Activin A (50 ng/mL) for 24 hours; gene expression normalized to Gapdh (n=4). FIG. 11F shows an immunoblot of lysates from ctrl- and ACVR1/INHBA-transduced mouse BMSCs stimulated with Activin A (50 ng/ml) at different time points. Lysates were immunoblotted for phospho-Smad1/5; Hsp90 was used as a loading control.

FIGS. 12A-12F show that local delivery of an exemplary nucleic acid comprising a first nucleic acid sequence encoding an inhibitory nucleic acid that targets an INHBA transcript; a second nucleic acid sequence encoding a wild-type ACVR1 protein; and a third nucleic acid sequence encoding an inhibitory nucleic acid that targets a mutant ACVR1 transcript prevents trauma-induced HO in FOP mice. FIG. 12A depicts a diagram of the study design and treatment methods. Either an AAV9 carrying gfp.MIR or an AAV9 carrying ACVR1/INHBA.MIR was injected transdermally (t.d) into the gastrocnemius muscle of 8-week-old ACVR1^{R206H/+}; Pdgfrα-GFP mice 3 days prior to pinch injury. 4 weeks later. ACVR1^{206H}, ACVR1^{Opt}, and INHBA expression was assessed by RT-PCR (FIG. 12B, n=4), and HO was assessed in the injured muscle by X-ray (FIG. 12C; arrow indicates heterotopic bone) and microCT (FIG. 12D). FIG. 12D shows 3D reconstruction images (left) and quantification of HO volume (right, n=?˜10). The frequency of GFP⁺Scal⁺CD31⁻CD45⁻ FAPs in the injured muscle was assessed by flow cytometry (FIG. 12E, n=4), or Pdgfrα-GFP FAPs were FACS sorted from the injured muscle and subjected to RT-PCR analysis to measure expression of Id1, Sox9, and Colla (FIG. 12F. n=4).

FIGS. 13A-13E show that local delivery of an exemplary nucleic acid comprising a first nucleic acid sequence encoding an inhibitory nucleic acid that targets an INHBA transcript; a second nucleic acid sequence encoding a wild-type ACVR1 protein; and a third nucleic acid sequence encoding an inhibitory nucleic acid that targets a mutant ACVR1 transcript suppresses progression of traumatic HO in FOP mice. FIG. 13A depicts a diagram of the study design and treatment methods. Pinch injury was performed on the gastrocnemius muscle of 8-week-old ACVR1^{R206H/+} mice and 1, 3, and 6 days later, 5×10¹² vg/kg of AAV9 carrying gfp.MIR or ACVR1/INHBA.MIR was injected t.d. into the injured muscle. ACVR1^{R206H}, ACVR1^{Opt}, and INHBA expression was assessed by RT-PCR 4 weeks post-injury (FIG. 13B, n=4). HO in the injured muscle was assessed by X-ray (FIG. 13C, top; arrows indicate heterotopic bone), microCT (FIG. 13C, bottom), and histology on a paraffin section of AAV-treated hindlimbs (FIG. 13D, n=9˜10). 3D reconstruction images and quantification of HO volume are shown (FIGS. 13C-13D, respectively). Longitudinal sections of the injured muscle were stained with alcian blue/hematoxylin/orange G (FIG. 13E). HB=heterotopic bone; F=fibrotic tissue: C=chondrogenic analgen; M=skeletal muscle. Scale bars=100 μm. ***, P<0.0001 by one-way ANOVA test.

### DETAILED DESCRIPTION

Aspects of the disclosure relate to methods and compositions for treating fibrodysplasia ossificans progressiva (FOP) and associated flare-up conditions (e.g., heterotopic ossification). The disclosure is based, in part, on compositions (e.g., compositions comprising one or more nucleic acid sequences, vectors, rAAVs, etc.) that reduce the expression of Activin A protein (e.g., via reduction of expression of INHBA transcripts). In some embodiments, the compositions disclosed herein reduce the expression of a mutated activin A receptor (e.g., ACVR1^{R206H}), alone or in combination with reduction of activin A expression. In some embodiments, the compositions disclosed herein increase the expression of a wild-type ACVR1 receptor (e.g., a codon-optimized ACVR1 receptor), alone or in combination with reduction of INHBA (which encodes a subunit of the homodimeric Activin A protein) and/or ACVR1^{R206H} expression. In some embodiments, the compositions disclosed herein at least inhibit heterotopic ossification (HO) and/or the flare up conditions when delivered to an affected subject. Accordingly, methods and compositions described by the disclosure are useful, in some embodiments, for the treatment of diseases and disorders associated with FOP.

Compositions and methods for delivering a nucleic acid (e.g., a nucleic acid encoding an inhibitory RNA, such as an amiRNA, shRNA, miRNA, etc.) to a subject are provided in the disclosure. The compositions typically comprise an isolated nucleic acid encoding one or more nucleic acid sequences (also referred to as one or more transgenes) (e.g., a protein, an inhibitory nucleic acid, etc.) capable of modulating genes associated with bone metabolism (e.g., INHBA and ACVR1) and/or treating FOP. For example, in some embodiments, a nucleic acid of the disclosure reduces expression of a target protein, such as a target protein associated with promoting bone formation. In some embodiments, a nucleic acid of the disclosure reduces expression of a target protein associated with aberrant signaling in FOP. For example, in some embodiments, a nucleic acid of the disclosure reduces expression of Activin A and/or ACVR1^{R206H}. In some embodiments, a nucleic acid of the disclosure encodes a wild-type ACVR1. In some embodiments, a transgene simultaneously reduces expression of Activin A and/or ACVR1^{R206H} (e.g., using genetic knockdown strategics) while simultaneously providing a wild-type ACVR1 (e.g., using genetic replacement strategies).

### Inhibitory Nucleic Acids

In some embodiments, the present disclosure provides a nucleic acid comprising an inhibitory nucleic acid sequence targeting an INHBA transcript (e.g., a gene transcript encoded by or within NCBI Gene ID: 3624), which encodes inhibin beta A (Activin A). In some embodiments, an inhibitory nucleic acid sequence comprises a region of complementarity with an INHBA mRNA transcript. In some embodiments, an INHBA mRNA transcript comprises or consists of the nucleic acid sequence set forth in NCBI Reference Sequence: NM_002192.4.

In some embodiments, the present disclosure provides a nucleic acid comprising an inhibitory nucleic acid sequence targeting a mutant ACVR1 gene transcript (e.g., a gene transcript encoded by or within NCBI Gene ID: 90). In some embodiments, the present disclosure provides a nucleic acid comprising an inhibitory nucleic acid sequence targeting a mutant ACVR1^{R206H} gene transcript having a substitution mutation at amino acid position 206 (Arginine to Histidine) (e.g., a gene transcript encoded by or within NCBI Gene ID: 90). In some embodiments, an inhibitory nucleic acid sequence comprises a region of complementarity with a mutant ACVR1 mRNA transcript. In some embodiments, a mutant ACVR1 mRNA transcript (e.g., an ACVR1^{R206H} mRNA transcript) comprises a nucleotide substitution at position 617 of a wild-type ACVR1 sequence (e.g., a wild-type ACVR1 sequence comprising or consisting of the nucleic acid sequence set forth in NCBI Reference Sequence: NM_001105.5, NM_001111067.4, NM_001347663.1. NM_001347664.1. NM_001347665.1. NM_001347666.1 or NM_001347667.2). In some embodiments, a mutant ACVR1 sequence comprises or consists of the nucleic acid sequence set forth in NCBI Reference Sequence: NM_001111067.4:c.617G>A.

In some embodiments, the present disclosure provides inhibitory nucleic acids (e.g., miRNA, artificial microRNA (amiRNA), dsRNA, siRNA, shRNA, etc.). In some embodiments, an inhibitory nucleic acid targets an INHBA transcript. In some embodiments, an inhibitory nucleic acid targets a mutant ACVR1 transcript (e.g., an ACVR1^{R206H} transcript).

Generally, an inhibitory nucleic acid specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a target gene. As used herein “continuous bases” refers to two or more nucleotide bases that are covalently bound (e.g., by one or more phosphodiester bond, etc.) to each other (e.g., as part of a nucleic acid molecule). In some embodiments, an inhibitory nucleic acid that targets an INHBA transcript specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of an INHBA transcript. In some embodiments, an inhibitory nucleic acid that targets a mutant ACVR1 transcript specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a mutant ACVR1 transcript. In some embodiments, an inhibitory nucleic acid that targets an ACVR1^{R206H} transcript specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of an ACVR1^{R206H} transcript.

In some embodiments, an inhibitory nucleic acid that targets an INHBA transcript comprises one or more inhibitory nucleic acid sequences provided in Table 1. In some embodiments, an inhibitory nucleic acid that targets an INHBA transcript comprises or consists of the nucleic acid sequence of any one of SEQ ID NOs: 41-45. In some embodiments, an inhibitory nucleic acid that targets an INHBA transcript comprises or consists of the nucleic acid sequence of SEQ ID NO: 41. In some embodiments, an inhibitory nucleic acid that targets an INHBA transcript comprises or consists of the nucleic acid sequence of SEQ ID NO: 42. In some embodiments, an inhibitory nucleic acid that targets an INHBA transcript comprises or consists of the nucleic acid sequence of SEQ ID NO: 43. In some embodiments, an inhibitory nucleic acid that targets an INHBA transcript comprises or consists of the nucleic acid sequence of SEQ ID NO: 44. In some embodiments, an inhibitory nucleic acid that targets an INHBA transcript comprises or consists of the nucleic acid sequence of SEQ ID NO: 45.

In some embodiments, an inhibitory nucleic acid that targets a mutant ACVR1 transcript (e.g., an ACVR1^{R206H} transcript) comprises or consists of the nucleic acid sequence of SEQ ID NO: 46. In some embodiments, an inhibitory nucleic acid that targets a mutant ACVR1 transcript (e.g., an ACVR1^{R206H} transcript) comprises or consists of the nucleic acid sequence of tgtaatctggtgagccactgt (SEQ ID NO: 46).

TABLE 1
Exemplary inhibitory nucleic acid sequences for
targcting an INHBA transcript

| | | SEQ | artificial miRNA sequence | SEQ |
|---|---|---|---|---|
| | Inhibitory nucleic | ID | comprising an inhibitory | ID |
| | acid sequence | NO: | nucleic acid sequence | NO: |
| INHBA | tctttcttcttcttcttgccc | 41 | GGCAGCCTTGGAGTGGGTTCCTGCCCCCTCG | 3 |
| #1 | | | GGCACACAAACAGAGCTGAAGACCACCCTG | |
| | | | GGCACCTCCTTGGCTGGCCGCATACCTCCTG | |
| | | | GCGGGCAGCTGTGtctttcttcttcttcttgcccTG | |
| | | | TTCTGGTGGTACCCAGGGCAAGATCAGGAAGAAAG | |
| | | | ACACAGAGGCCTGCCTGGCCCTCGAGAGAC | |
| | | | TGCCCTGACTGAAGGCCCTATCAGGTGGGGG | |
| | | | AGGGGATCCTGATAGAGGGCACTGCTGCCAC | |
| | | | TGTTGGGGCCCAAG | |
| INHBA | tttgccactgtcttctctgga | 42 | GGCAGCCTTGGAGTGGGTTCCTGCCCCCTCG | 4 |
| #2 | | | GGCACACAAACAGAGCTGAAGACCACCCTG | |
| | | | GGCACCTCCTTGGCTGGCCGCATACCTCCTG | |
| | | | GCGGGCAGCTGTGtttgccactgtcttctctggaT | |
| | | | GTTCTGGTGGTACCCATCCAGAGATCATAGTGGCAA | |
| | | | ACACAGAGGCCTGCCTGGCCCTCGAGAGAC | |
| | | | TGCCCTGACTGAAGGCCCTATCAGGTGGGGG | |
| | | | AGGGGATCCTGATAGAGGGCACTGCTGCCAC | |
| | | | TGTTGGGGCCCAAG | |
| INHBA | ttcctttcaagtatctctcct | 43 | GGCAGCCTTGGAGTGGGTTCCTGCCCCCTCG | 5 |
| #3 | | | GGCACACAAACAGAGCTGAAGACCACCCTG | |
| | | | GGCACCTCCTTGGCTGGCCGCATACCTCCTG | |
| | | | GCGGGCAGCTGTGttcctttcaagtatctctcctT | |
| | | | GTTCTGGTGGTACCCAAGGAGAGAATCCTGAAAGG | |
| | | | AACACAGAGGCCTGCCTGGCCCTCGAGAGA | |
| | | | CTGCCCTGACTGAAGGCCCTATCAGGTGGGG | |
| | | | GAGGGGATCCTGATAGAGGGCACTGCTGCC | |
| | | | ACTGTTGGGGCCCAAG | |
| INHBA | Tgatctccgaggtctgctc | 44 | GGCAGCCTTGGAGTGGGTTCCTGCCCCCTCG | 6 |
| #4 | ca | | GGCACACAAACAGAGCTGAAGACCACCCTG | |
| | | | GGCACCTCCTTGGCTGGCCGCATACCTCCTG | |
| | | | GCGGGCAGCTGTGtgatctccgaggtctgctccaT | |
| | | | GTTCTGGTGGTACCCATGGAGCAGTGCCCGGAGAT | |
| | | | CACACAGAGGCCTGCCTGGCCCTCGAGAGA | |
| | | | CTGCCCTGACTGAAGGCCCTATCAGGTGGGG | |
| | | | GAGGGGATCCTGATAGAGGGCACTGCTGCC | |
| | | | ACTGTTGGGGCCCAAG | |
| INHBA | ttttgatgatgttttgaccat | 45 | GGCAGCCTTGGAGTGGGTTCCTGCCCCCTCG | 7 |
| #5 | | | GGCACACAAACAGAGCTGAAGACCACCCTG | |
| | | | GGCACCTCCTTGGCTGGCCGCATACCTCCTG | |
| | | | GCGGGCAGCTGTGttttgatgatgttttgaccatT | |
| | | | GTTCTGGTGGTACCCAATGGTCAATTCGTCATCAAA | |
| | | | ACACAGAGGCCTGCCTGGCCCTCGAGAGAC | |
| | | | TGCCCTGACTGAAGGCCCTATCAGGTGGGGG | |
| | | | AGGGGATCCTGATAGAGGGCACTGCTGCCAC | |
| | | | TGTTGGGGCCCAAG | |
| INHBA | Tctttcttcttcttcttgccc | 41 | GGCAGCCTTGGAGTGGGTTCCTGCCCCCTCG | 8 |
| #6 | | | GGCACACAAACAGAGCTGAAGACCACCCTG | |
| | | | GGCACCTCCTTGGCTGGCCGCATACCTCCTG | |
| | | | GCGGGCAGCTGTGtctttcttcttcttcttgcccTG | |
| | | | TTCTGGTGGTACCCAGGGCAAGATCAGGAAGAAAG | |
| | | | ACACAGAGGCCTGCCTGGCCCTCGAGAGAC | |
| | | | TGCCCTGACTGAAGGCCCTATCAGGTGGGGG | |
| | | | AGGGGATCCTGATAGAGGGCACTGCTGCCAC | |
| | | | TGTTGGGGCCCAAG | |

**Ts may be substituted w/ Us

In some embodiments, a microRNA (miRNA) or an artificial miRNA (amiRNA) that targets an INHBA transcript comprises one or more inhibitory nucleic acid sequences provided in Table 1. In some embodiments, a miRNA or an amiRNA that targets an INHBA transcript comprises or consists of the nucleic acid sequence of any one of SEQ ID NOs: 41-45. In some embodiments, a miRNA or an amiRNA that targets an INHBA transcript comprises or consists of the nucleic acid sequence of SEQ ID NO: 41. In some embodiments, a miRNA or an amiRNA that targets an INHBA transcript comprises or consists of the nucleic acid sequence of SEQ ID NO: 42. In some embodiments, a miRNA or an amiRNA that targets an INHBA transcript comprises or consists of the nucleic acid sequence of SEQ ID NO: 43. In some embodiments, a miRNA or an amiRNA that targets an INHBA transcript comprises or consists of the nucleic acid sequence of SEQ ID NO: 44. In some embodiments, a miRNA or an amiRNA that targets an INHBA transcript comprises or consists of the nucleic acid sequence of SEQ ID NO: 45. In some embodiments, a miRNA or an amiRNA that targets an INHBA transcript comprises or consists of the nucleic acid sequence of any one of SEQ ID NO.: 3-8. In some embodiments, a miRNA or an amiRNA that targets an INHBA transcript comprises or consists of the nucleic acid sequence of SEQ ID NO: 3. In some embodiments, a miRNA or an amiRNA that targets an INHBA transcript comprises or consists of the nucleic acid sequence of SEQ ID NO: 4. In some embodiments, a miRNA or an amiRNA that targets an INIIBA transcript comprises or consists of the nucleic acid sequence of SEQ ID NO: 5. In some embodiments, a miRNA or an amiRNA that targets an INHBA transcript comprises or consists of the nucleic acid sequence of SEQ ID NO: 6. In some embodiments, a miRNA or an amiRNA that targets an INHBA transcript comprises or consists of the nucleic acid sequence of SEQ ID NO: 7. In some embodiments, a miRNA or an amiRNA that targets an INHBA transcript comprises or consists of the nucleic acid sequence of SEQ ID NO: 8.

In some embodiments, a miRNA or an amiRNA that targets a mutant ACVR1 transcript (e.g., an ACVR1^{R206H} transcript) comprises or consists of the nucleic acid sequence of SEQ ID NO: 35. In some embodiments, a miRNA or an amiRNA that targets a mutant ACVR1 transcript (e.g., an ACVR1^{R206H} transcript) comprises or consists of the nucleic acid sequence of

(SEQ ID NO: 35)
agggctctgcgtttgctccaggtagtccgctgctcccttgggcctgggcc
cactgacagccctggtgcctctggccggctgcacacctcctggcgggcag
ctgtgtgtaatctggtgagccactgttgttctggcaatacctgacagtgg
cagatcagattacacacggaggcctgccctgactgcccacggtgccgtgg
ccaaagaggatctaagggcaccgctgagggcctacctaaccatcgtgggg
aataaggacagtgtcaccc

A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length.

As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014) Methods Mol. Biol. 1062:211-224. For example, in some embodiments an artificial miRNA comprises a miR-155 pri-miRNA backbone into which a sequence encoding an inhibitory miRNA has been inserted in place of the endogenous miR-155 mature miRNA-encoding sequence. In some embodiments, miRNA (e.g., an artificial miRNA) as described by the disclosure comprises a miR-33 backbone sequence, a miR-155 backbone sequence, a miR-30 backbone sequence, a miR-30A backbone sequence, a miR-64 backbone sequence, or a miR-122 backbone sequence.

In some embodiments, an artificial microRNA is between 6-50 nucleotides in length. In some embodiments, an artificial microRNA is between 8-24 nucleotides in length. In some embodiments, an artificial microRNA is between 12-36 nucleotides in length. In some embodiments, an artificial microRNA is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an inhibitory nucleic acid sequence decreases expression of a target gene by between 50% and 99% (e.g., any integer between 50% and 99%, inclusive). In some embodiments, an inhibitory nucleic acid sequence decreases expression of a target gene by between 75% and 90%. In some aspects, an inhibitory nucleic acid sequence decreases expression of a target gene by between 80% and 99%. In some embodiments, an inhibitory nucleic acid sequence targeting an INHBA transcript decreases expression of an INHBA transcript by between 50% and 99% (e.g., any integer between 50% and 99%, inclusive). In some embodiments, an inhibitory nucleic acid sequence targeting an INHBA transcript decreases expression of an INHBA transcript by between 75% and 90%. In some aspects, an inhibitory nucleic acid sequence targeting an INHBA transcript decreases expression of an INHBA transcript by between 80% and 99%. In some embodiments, an inhibitory nucleic acid sequence targeting a mutant ACVR1 transcript (e.g., an ACVR1^{R206H} transcript) decreases expression of a mutant ACVR1 transcript (e.g., an ACVR1^{R206H} transcript) by between 50% and 99% (e.g., any integer between 50% and 99%, inclusive). In some embodiments, an inhibitory nucleic acid sequence targeting a mutant ACVR1 transcript (e.g., an ACVR1^{R206H} transcript) decreases expression of a mutant ACVR1 transcript (e.g., an ACVR1^{R206H} of transcript) by between 75% and 90%. In some aspects, an inhibitory nucleic acid sequence targeting a mutant ACVR1 transcript (e.g., an ACVR1^{R206H} transcript) decreases expression of a mutant ACVR1 transcript (e.g., an ACVR1^{R206H} transcript) by between 80% and 99%.

### Nucleic Acid Sequences Encoding a Protein

In some embodiments, the present disclosure provides nucleic acid sequences encoding an ACVR1 protein (e.g., a wild-type ACVR1 protein). In some embodiments, a nucleic acid sequence encoding an ACVR1 protein is flanked by adeno-associated virus (AAV) inverted terminal repeats (ITRs). An ACVR1 protein may be a human ACVR1 protein (e.g., comprising or consisting of the amino acid sequence of SEQ ID NO: 25). In some embodiments, an ACVR1 protein is a wild-type ACVR1 protein. In some embodiments, a wild-type ACVR1 protein comprises the amino acid sequence of SEQ ID NO: 25. In some embodiments, a wild-type ACVR1 protein has an arginine at position 206 (numbering relative to SEQ ID NO: 25) and comprises at least 90% identity, at least 95% identity, at least 97% identity, at least 98% identity, or at least 99% identity to the amino acid sequence of SEQ ID NO: 25. In some embodiments, a wild-type ACVR1 consists of the sequence as set forth in SEQ ID NO: 25.

A nucleic acid encoding an ACVR1 protein (e.g., a wild-type ACVR1 protein) may be a codon-optimized sequence. In some embodiments, a codon-optimized ACVR1 sequence encodes a wild-type ACVR1 protein. In some embodiments, the codon composition of an ACVR1 gene can be improved or optimized for expression in a subject (e.g., a human subject) without altering the encoded amino acid sequence of ACVR1. In some embodiments, a codon-optimized ACVR1 comprises a coding sequence as set forth in SEQ ID NO: 28. In some embodiments, a codon-optimized nucleic acid sequence encoding ACVR1 comprises at least 80% identity, at least 85% identity, at least 90% identity, at least 95% identity, at least 97% identity, at least 98% identity, or at least 99% identity to the nucleic acid sequence set forth in SEQ ID NO: 28. In some embodiments, a codon-optimized ACVR1 consists of the sequence as set forth in SEQ ID NO: 28.

As disclosed herein. “identity” of sequences refers to the measurement or calculation of the percent of identical matches between two or more sequences with gap alignments addressed by a mathematical model, algorithm, or computer program that is known to one of ordinary skill in the art. The percent identity of two sequences (e.g., nucleic acid or amino acid sequences) may, for example, be determined using Basic Local Alignment Search Tool (BLAST®) such as NBLAST® and XBLAST® programs (version 2.0). Alignment technique such as Clustal Omega may be used for multiple sequence alignments. Other algorithms or alignment methods may include but are not limited to the Smith-Waterman algorithm, the Needleman-Wunsch algorithm, or Fast Optimal Global Sequence Alignment Algorithm (FOGSAA).

In some embodiments, the present disclosure provides nucleic acid sequences encoding a gene associated with tumor necrosis factor (TNF) signaling. In some embodiments, a nucleic acid sequence encodes a soluble TNF receptor type 2 (sTNFR2) protein (e.g., a protein encoded by a TNFRSF1B gene). In some embodiments, a sTNFR2 protein comprises or consists of the sequence set forth in SEQ ID NO: 36. In some embodiments, a nucleic acid sequence comprises a nucleic acid sequence encoding a sTNFR2 protein flanked by AAV ITRs. A sTNFR2 protein may be a human sTNFR2 protein.

In some embodiments, the present disclosure provides a nucleic acid sequence encoding a gene associated with cytokine (e.g., interleukin-1 [IL-1]) signaling. Without wishing to be bound by theory, soluble IL-1Rα (sIL-1Rα) is an IL-1 receptor antagonist that has been shown to effectively slow the progression of FOP (Anakinra [Kineret]). In some embodiments, a nucleic acid sequence encodes a sIL-1Rα protein (e.g., a protein encoded by an IL1RN gene). In some embodiments, a sIL-1Rα protein comprises or consists of the sequence set forth in SEQ ID NO: 37.

A region comprising a nucleic acid sequence (also referred to herein as a “transgene”) encoding a protein (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA (e.g., an artificial miRNA targeting INHBA) may be positioned in an intron of the transgene and a nucleic acid sequence encoding a second miRNA (e.g., an artificial miRNA targeting ACVR1^{R206H}) may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, an enhancer, etc.). Expression control sequences include appropriate transcription initiation, termination, promoter and enhancer sequences; efficient RNA processing signals such as splicing and polyadenylation (poly-A) signals; sequences that stabilize cytoplasmic mRNA: sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the machinery of the cell, or introduced synthetic machinery, required to initiate the specific transcription of a gene. The phrases “operatively positioned,”. “operatively linked”, “under control” or “under transcriptional control” mean that the promoter is in the correct location and orientation in relation to the nucleic acid to control RNA polymerase initiation and expression of the gene.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, a transgene described herein comprises a Kozak sequence. A Kozak sequence is a nucleic acid motif comprising a consensus sequence that is found in eukaryotic mRNA and plays a role in initiation of protein translation. In some embodiments, the Kozak sequence is positioned between the intron and the transgene encoding transgene described herein.

For nucleic acids encoding proteins, a polyadenylation (poly-A) sequence generally is inserted following the transgene sequences and before the 3′ AAV ITR sequence. A rAAV construct useful in the present disclosure may also contain an intron, desirably located between the promoter/enhancer sequence and the transgene. As disclosed herein, one possible intron sequence is derived from SV-40 and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contains more than one polypeptide chains. Selection of these and other common vector elements are conventional and many such sequences are available (see, e.g., Sambrook et al., and references cited therein at, for example, pages 3.18 3.26 and 16.17 16.27 and Ausubel et al., Current Protocols in Molecular Biology. John Wiley & Sons, New York, 1989). In some embodiments, a Foot and Mouth Disease Virus 2A sequence is included in polyprotein: this is a small peptide (approximately 18 amino acids in length) that has been shown to mediate the cleavage of polyproteins (Ryan, M D et al., EMBO, 1994; 4, 928-933; Mattion, N M et al., J Virology. November 1996; p. 8124-8127; Furler. S et al., Gene Therapy, 2001; 8: 864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO. 1994; 4: 928-933; Mattion, N M et al., J Virology. November 1996; p. 8124-8127; Furler, S ct al., Gene Therapy, 2001; 8: 864-873; and Halpin, C et al., The Plant Journal. 1999; 4:453-459; de Felipe. P et al., Gene Therapy, 1999; 6: 198-208; de Felipe. P et al., Human Gene Therapy. 2000; 11:1921-1931; and Klump, H et al., Gene Therapy, 2001; 8:811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (sec. e.g., Boshart et al., Cell. 41:521-530 (1985)), the SV40 promoter, the dihydrofolate reductase promoter, the β-actin promoter (e.g., chicken β-actin promoter), the phosphoglycerol kinase (PGK) promoter, and the EF1α promoter [Invitrogen]. In some embodiments, a promoter is an enhanced chicken β-actin promoter. In some embodiments, the chicken β-actin promoter comprises or consists of the sequence set forth in SEQ ID NO: 30. In some embodiments, a promoter is a U6 promoter. In some embodiments, the enhancer is a CMV enhancer. In some embodiments, the CMV enhancer comprises or consists of the sequence set forth in SEQ ID NO: 31.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter (Sandig et al., Gene Ther., 3:1002-9 (1996)); alpha-fetoprotein (AFP) promoter, (Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal promoters such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron. 15:373-84 (1995)), among others which will be apparent to the skilled artisan.

In some embodiments, a tissue-specific promoter is a bone tissue-specific promoter. Examples of bone tissue-specific promoters include but are not limited to promoters of osterix, osteocalcin, type 1 collagen α1, DMP1, cathepsin K, Rank, Runx2, Prx1, Sox9, etc. In some embodiments, a tissue-specific promoter is a skeletal muscle-specific promoter. Examples of skeletal muscle-specific promoters include but are not limited to human skeletal actin (HSA) or alpha skeletal actin (ACTA1) or muscle creatine kinase (MCK), or engineered muscle specific promoters such as the CK1-7 collection or the hybrid MHCK7, or the C5-12 collection.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., artificial miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is an RNA polymerase III (polIII) promoter sequence. Non-limiting examples of polIII promoter sequences include U6 and HI promoter sequences. In some embodiments, the second promoter sequence (e.g., the promoter sequence driving expression of the inhibitory RNA) is an RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polll promoter sequence drives expression of a protein coding region.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the eedysone insect promoter (No et al., Proc. Natl. Acad. Sei. USA. 93:3346-3351 (1996)). the tetracycline-repressible system (Gossen et al., Proc. Natl. Acad. Sci. USA, 89:5547-5551 (1992)), the tetracycline-inducible system (Gossen et al., Science. 268: 1766-1769 (1995), see also Harvey et al., Curr. Opin. Chem. Biol., 2:512-518 (1998), the RU486-inducible system (Wang et al., Nat. Biotech . . . 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, an inducible promoter drives expression of a transgene under “flare-up” conditions (“flare-up responsive promoter”). A flare-up responsive promoter, as used herein, refers to a promoter sequence that drives expression of a transgene in response to signaling associated with FOP and/or its associated symptoms (e.g., BMP signaling). In some embodiments, a flare-up responsive promoter drives expression of a transgene in response to increased BMP signaling and/or NF-κB signaling. In some embodiments, a flare-up responsive promoter comprises a first portion comprising a NF-κB promoter and a second portion comprising a bone morphogenic protein (BMP) response element promoter (pBRE). In some embodiments, the NF-κB promoter comprises or consists of the sequence set forth in SEQ ID NO: 34. In some embodiments, the pBRE sequence comprises or consists of the sequence set forth in SEQ ID NO: 38. In some embodiments, the flare-up responsive promoter comprises or consists of the sequence as set forth in SEQ ID NO: 21. In some embodiments, the first promoter sequence (e.g., the promoter driving expression of the protein coding region) is a pBRE sequence and the second promoter sequence (e.g., a second promoter sequence operably linked to the inhibitory RNA encoding region) is a NF-κB promoter sequence. In some embodiments, a flare-up (e.g., inflammation) induces expression of a transgene, such as, for example, a transgene encoding ACVR1, sTNFR2. sIL-1Rα, or one or more inhibitory nucleic acids (e.g., amiRNAs).

In some embodiments, a nucleic acid comprises a transgene encoding one or more proteins that act as a “trap” for Activin A. As used herein an “Activin A trap” refers to a protein that competes with another protein for Activin A binding. Thus, in some embodiments, an Activin A trap is used to bind Activin A such that it may not hind another protein (e.g., an ACVR1 and/or ACVR1^{R206H} receptor). In some embodiments, an Activin A trap is a kinase-dead ACVR receptor (e.g., a kinase-dead human ACVR2A [hACVR2A Kinase-del] and/or ACVR2B [hACVR2B Kinase-del|receptor). As used herein, “kinase-dead” refers to a mutant form of a protein that normally has kinase activity (i.e., a “kinase”), wherein its mutations render it unable to catalyze the transfer of phosphate groups to a substrate. In some embodiments, a nucleic acid comprises a transgene encoding an Activin A trap comprising or consisting of a sequence set forth in SEQ ID NOS: 39 or 40. In some embodiments, an Activin A trap is provided to a cell with aberrant Activin A signaling (e.g., a FOP bone cell).

In some embodiments, a nucleic acid comprises a transgene comprising: (i) a first nucleic acid sequence encoding an ACVR1 protein (e.g., a human ACVR1 protein); and (ii) a second nucleic acid sequence encoding one or more inhibitory nucleic acids targeting an INHBA and/or an ACVR1^{R206H} sequence (e.g., a miRNA or ami-RNA that targets INHBA for inhibition comprises a antisense sequence comprising or consisting of a nucleic acid sequence of any one of SEQ ID NOs: 37-42). In some embodiments, the transgene is flanked by adeno-associated virus (AAV) inverted terminal repeats (ITRs) (e.g., AAV2 ITRs).

In some embodiments, the transgene comprises a 3′-untranslated region (3′-UTR). In some embodiments, the disclosure relates to isolated nucleic acids comprising a transgene which comprises one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in the 3′ untranslated region (3′-UTR) of a transgene, for example between the last codon of a nucleic acid sequence encoding a transgene and a poly A sequence.

In some embodiments, a transgene comprises one or more (e.g., 1, 2, 3, 4, 5, or more) miRNA binding sites that de-target expression of the transgene from specific cell types. In some embodiments, a transgene comprises one or more miRNA binding sites that de-target expression of the transgene from liver cells (e.g., cholangiocytes, hepatocytes, Kupffer cells, liver stellate cells, etc). In some embodiments, incorporation of miRNA binding sites for liver-associated miRNAs de-targets transgene expression from the liver and thus reduces or eliminates off-target effects in a subject (see, e.g., Xic J. Su Q. Xie Q, Mueller C, Zamore P, Gao G. MicroRNA regulated tissue specific transduction by rAAV vector. American Society of Gene Therapy Annual Meeting. San Diego; 2009). In some embodiments, a transgene comprises one or more miR-122 binding sites. In some embodiments, a miR-122 binding site comprises or consists of the sequence set forth in SEQ ID NO: 32. In some embodiments, a transgene comprises one or more miR-208a binding sites. In some embodiments, a miR-208a binding site comprises or consists of the sequence set forth in SEQ ID NO: 33. In some embodiments, a miRNA binding site that de-targets expression from a specific cell type comprises or consists of the sequence set forth in SEQ ID NO: 24.

As used herein, a “liver-associated miRNA” is a miRNA preferentially expressed in a liver cell, such as a hepatocyte. In some embodiments, a liver-associated miRNA is a miRNA expressed in liver cells that exhibits at least a 2-fold, 3-fold, 4-fold. 5-fold, 6-fold, 7-fold, 8-fold, 9-fold, 10-fold, or higher level of expression in a liver cell compared with a non-liver cell (e.g., a control cell, such as a HEK293 cell, a HeLa cell, etc.). In some embodiments, the liver-associated miRNA is selected from miR-122, miR-194, miR-192, and miR-29.

Recombinant Adeno-Associated Viruses (rAAVs)

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. Recombinant AAV (rAAV) vectors are typically composed of, at a minimum (i) a transgene and its regulatory sequences, and (ii) 5′ and 3′ AAV inverted terminal repeats (ITRs). The transgene may comprise, as disclosed elsewhere herein, one or more regions that encode one or more proteins and/or inhibitory nucleic acids (e.g., shRNA, miRNAs, amiRNAs, etc.) comprising a nucleic acid that targets an endogenous mRNA of a subject. The transgene may also comprise a region encoding, for example, a protein and/or an expression control sequence (e.g., a promoter, a poly-A tail, etc.), as described elsewhere in the disclosure. In some embodiments, nucleic acid coding sequence is operatively linked to regulatory components in a manner which permits transgene transcription, translation, and/or expression in a cell of a target tissue.

In some aspects, the instant disclosure provides a vector comprising a single, cis-acting wild-type ITR. In some embodiments, the ITR is a 5′ ITR. In some embodiments, the ITR is a 3′ ITR. Another example of such a molecule employed in the present disclosure is a “cis-acting” plasmid containing the transgene, in which the selected transgene sequence and associated regulatory elements are flanked by the 5′ AAV ITR sequence and a 3′ hairpin forming RNA sequence. Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory. New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVmh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV9 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV3, AAV4, AAV5, AAV6, AAV6.2, AAV7, AAV8, AAVrh8, AAV9, AAVrh10. AAVrh39, AAVrh43, AAV2/2-66, AAV2/2-84, AAV2/2-125, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (WT) AAV TR at each end and a mutated TR (mTR) in the middle. The instant invention is based, in part, on the recognition that DNA fragments encoding RNA hairpin structures (e.g., shRNA, miRNA, and AmiRNA) can serve a function similar to a mutant inverted terminal repeat (mTR) during viral genome replication, generating self-complementary AAV vector genomes. For example, in some embodiments, the disclosure provides rAAV (e.g., self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, ami-RNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US 2003/0138772, the contents of which are incorporated herein by reference in their entirety). Typically, the methods involve culturing a host cell which contains a nucleic acid sequence encoding an AAV capsid protein: a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1. VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, or AAV.rh43 serotype. In some embodiments, an AAV capsid protein is of an AAV9 serotype.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative. a selected stable host cell may contain selected component(s) under the control of a constitutive promoter and other selected component(s) under the control of one or more inducible promoters. For example, a stable host cell may be generated which is derived from HEK293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See. e.g., Sambrook et al., Molecular Cloning: A Laboratory Manual, Cold Spring Harbor Press, Cold Spring Harbor, N. Y. Similarly, methods of generating rAAV virions are well known and the selection of a suitable method is not a limitation on the present disclosure. See, e.g., K. Fisher ct al., J. Virol., 70: 520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function, vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing. AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, ycast cell, insect cell (Sf9), or a mammalian (e.g., human, rodent, non-human primate, etc.) cell. In some embodiments, the mammalian cell is a HEK293 cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically active polypeptide or production of a biologically active nucleic acid such as an RNA, has been introduced.

In some aspects, the present disclosure provides a recombinant AAV comprising a capsid protein and an isolated nucleic acid comprising a first region encoding an AAV ITR and a second region comprising a transgene, wherein the transgene encodes an artificial microRNA. The artificial microRNA may decrease the expression of a target gene in a cell or tissue (e.g. skeletal muscles, tendons, and cartilage) or a subject. In some embodiments, the rAAV comprises an artificial microRNA that decreases the expression of ACVR1 in a cell, tissue, or a subject.

The foregoing methods for packaging recombinant vectors in desired AAV capsids to produce the rAAVs of the disclosure are not meant to be limiting and other suitable methods will be apparent to the skilled artisan.

### Modes of Administration and Compositions

The isolated nucleic acids, nucleic acid sequences, compositions comprising nucleic acid sequences, rAAVs, engineered T cells, and pharmaceutical compositions of the present disclosure may be delivered to a subject in accordance with any appropriate methods known in the art. For example, a nucleic acid sequence, isolated nucleic acid or rAAV may be preferably suspended in a physiologically compatible carrier (e.g., in a composition), and administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, a subject is an adult. In some embodiments, a subject is a juvenile or infant.

In some embodiments, the nucleic acid sequence, isolated nucleic acid or rAAV comprises a transgene encoding ACVR1 having a sequence that is at least 70%, 71%, 72%, 73%, 74%, 75%, 76%, 77%, 78%, 79%, 80%, 81%, 82%, 83%, 84%, 85%, 86%, 87%, 38%, 89%, 90%, 91%, 92%, 93%, 94%, 95%, 96%, 97%, 98%, or 99% identical, or is 100% identical, including all values in between, to SEQ ID NO: 28. In some embodiments, the nucleic acid sequence, isolated nucleic acid or rAAV comprises a transgene encoding ACVR1 having the sequence set forth in SEQ ID NO: 28 (or the complementary sequence thereof), or a portion thereof.

In some embodiments, the nucleic acid sequence, isolated nucleic acid or rAAV comprises an inhibitory nucleic acid (e.g., an artificial miRNA) targeting an INHBA transcript having a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, or is 100% identical, including all values in between, to any one of SEQ ID NOs: 41-45. In some embodiments, the nucleic acid sequence, isolated nucleic acid or rAAV comprises an inhibitory nucleic acid (e.g., an artificial miRNA) targeting a mutant ACVR1 transcript having a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, or is 100% identical, including all values in between, to any one of SEQ ID NOs: 41-45.

In some embodiments, the nucleic acid sequence, isolated nucleic acid or rAAV comprises a promoter sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical, or is 100% identical, including all values in between, to SEQ ID NO: 21. In some embodiments, the nucleic acid sequence, isolated nucleic acid or rAAV comprises a promoter sequence having the sequence set forth in SEQ ID NO: 21 (or the complementary sequence thereof), or a portion thereof.

Delivery of the compositions, nucleic acid sequences, isolated nucleic acids, or rAAVs to a mammalian subject may be by, for example, intramuscular injection or by administration into the bloodstream of the mammalian subject. Administration into the bloodstream may be by injection into a vein, an artery, or any other vascular conduit. In some embodiments, the compositions, nucleic acid sequences, isolated nucleic acids, or rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the skeletal muscle and/or to the connective tissues of a subject. Recombinant AAVs may be delivered directly to the skeletal muscle (e.g., the gastrocnemius muscle) and/or to the connective tissues by injection into, e.g., directly into the muscle or the tissue, via intrasynovial injection, knee injection, etc., with a needle, catheter or related device, using surgical techniques known in the art.

In some embodiments, compositions, nucleic acid sequences, isolated nucleic acids, or rAAVs as described in the disclosure are administered by interdermal delivery or intradermal delivery. The delivery procedures and methods can be any techniques that are known in the art and/or suitable for the present disclosure.

In some embodiments, compositions, nucleic acid sequences, isolated nucleic acids, or rAAVs as described in the disclosure are administered by microneedle drug delivery such as transdermal application. In some embodiments, compositions, nucleic acid sequences, isolated nucleic acids, or rAAVs as described in the disclosure are administered by the use of dermal patches for providing controlled delivery. A dermal patch, skin patch, or the like as used herein refers to a medicated adhesive patch that is placed on the skin to deliver a specific dose of a composition into the skin. Dermal or skin patches can include but are not limited to single-layer drug-in-adhesive, multi-layer drug-in-adhesive, reservoir, matrix, and vapor patches. Alternatively. or additionally, the rate can be controlled by either providing a rate controlling membrane and/or by dispersing the active ingredient in a polymer matrix and/or gel. In some embodiments, permeation enhancers can be used for enhancing the permeation of compositions, nucleic acid sequences, isolated nucleic acids, or rAAVs in the patch. In some embodiments, compositions, nucleic acid sequences, isolated nucleic acids, or rAAVs as described in the disclosure are administered by intravenous injection. In some embodiments, the compositions, nucleic acid sequences, isolated nucleic acids, or rAAVs are administered by intramuscular injection.

Aspects of the instant disclosure relate to compositions comprising a recombinant AAV comprising a capsid protein and a nucleic acid encoding a transgene, wherein the transgene comprises a nucleic acid sequence encoding one or more codon-optimized ACVR1 genes and/or an artificial miRNA, for example. In some embodiments, the nucleic acid further comprises one or more AAV ITRs. In some embodiments, a composition further comprises a pharmaceutically acceptable carrier. In some embodiments, compositions comprise a recombinant AAV comprising a capsid protein and a nucleic acid comprising a first region encoding an AAV ITR and a second region comprising a transgene, wherein the transgene encodes (i) an artificial microRNA that targets Activin A and/or a mutant ACVR1 (e.g., ACVR1^{R206H}, and (ii) a codon-optimized ACVR1.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the composition, nucleic acid sequence, isolated nucleic acid, or rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions, nucleic acid sequences, isolated nucleic acids, or rAAVs are administered in sufficient amounts to transfect the cells of a desired tissue and to provide sufficient levels of gene transfer and expression without undue adverse effects. Conventional and pharmaceutically acceptable routes of administration include, but are not limited to. direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of compositions, nucleic acid sequences, isolated nucleic acids, or rAAVs required to achieve a particular “therapeutic effect,” e.g., the units of dose in genome copies/per kilogram of body weight (GC/kg), will vary based on several factors including, but not limited to: the route of administration, the level of gene or RNA expression required to achieve a therapeutic effect, the specific disease or disorder being treated, and the stability of the gene or RNA product. One of skill in the art can readily determine a dose range to treat a patient having a particular disease or disorder based on the aforementioned factors, as well as other factors that are well known in the art.

In some embodiments, an “effective amount” of a substance is an amount sufficient to produce a desired effect (e.g., to transduce bone cells or bone tissue). In some embodiments, an effective amount of an isolated nucleic acid is an amount sufficient to transfect (or infect in the context of rAAV-mediated delivery) a sufficient number of target cells of a target tissue of a subject. In some embodiments, a target tissue is skeletal muscle or connective tissues. In some embodiments, an effective amount of an isolated nucleic acid (e.g., which may be delivered via an rAAV) may be an amount sufficient to have a therapeutic benefit in a subject, e.g., to inhibit heterotopic ossification, to improve flare-up conditions, etc. The effective amount will depend on a variety of factors such as, for example, the species, age, weight, health of the subject, and the tissue to be targeted, and may thus vary among subject and tissue as described elsewhere in the disclosure. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments. 10¹² or 10¹³ rAAV genome copies is effective to target bone tissue.

In some embodiments, a dose of compositions, nucleic acid sequences, isolated nucleic acids, or rAAVs is administered to a subject no more than once per calendar day (e.g., a 24-hour period). In some embodiments, a dose is administered to a subject no more than once per 2, 3, 4, 5, 6, or 7 calendar days. In some embodiments, a dose is administered to a subject no more than once per calendar week (e.g., 7 calendar days). In some embodiments, a dose is administered to a subject no more than bi-weekly (e.g., once in a two calendar week period). In some embodiments, a dose is administered to a subject no more than once per calendar month (e.g., once in 30 calendar days). In some embodiments, a dose is administered to a subject no more than once per six calendar months. In some embodiments, a dose is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. (See, e.g., Wright F R. et al., Molecular Therapy (2005) 12, 171-178, the contents of which are incorporated herein by reference.)

In some embodiments, administering the isolated nucleic acid, the rAAV, and/or the vector as disclosed herein results in a decrease of the human Activin A protein by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, administering the isolated nucleic acid, the rAAV, the vector as disclosed herein results in a decrease of the human Activin A protein by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, and/or the vector as disclosed herein results in a decrease of mutant ACVR1 protein comprising a single base mutation of guanine to adenine at position 206 of the sequence of the wild type ACVR1 (ACVR1^{R206H}) by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, administering the isolated nucleic acid, the rAAV, the vector as disclosed herein results in a decrease of the human ACVR1^{R206H} protein by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, and/or the vector as disclosed herein results in an increase of the wild-type human ACVR1 protein by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, administering the isolated nucleic acid, the rAAV, the vector as disclosed herein results in an increase of the human ACVR1 protein by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering compositions, nucleic acid sequences, isolated nucleic acids, or rAAVs as disclosed herein results in an increase of an Activin A trap (e.g., hACVR2A Kinase-del and/or hACVR2B Kinase-del) by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, administering compositions, nucleic acid sequences, isolated nucleic acids, or rAAVs as disclosed herein results in an increase of an Activin A trap by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering compositions, nucleic acid sequences, isolated nucleic acids, or rAAVs as disclosed herein ameliorates heterotopic bone formation or heterotopic ossification by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the isolated nucleic acid, the rAAV, and/or the vector as disclosed herein ameliorates severe osteoarthritis by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the compositions, nucleic acid sequences, isolated nucleic acids, or rAAVs as disclosed herein decreases heterotopic bone mass by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the compositions, nucleic acid sequences, isolated nucleic acids, or rAAVs as disclosed herein decreases chondrogenic anlagen by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, administering the compositions, nucleic acid sequences, isolated nucleic acids, or rAAVs as disclosed herein decreases BMP-responsive genes by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control. In some embodiments, the BMP-responsive gene is ID1. In some embodiments, the BMP-responsive gene is MSX2. In some embodiments, the BMP-responsive gene is any gene that can be affected by the BMP signaling pathway.

As used herein, the improvement or stimulation is relative to a control. The control can be in a state that is prior to the administration of the isolated nucleic acid, the rAAV, and the vector. The improvement or stimulation is relative to a subject that has not been administered the compositions, nucleic acid sequences, isolated nucleic acids, or rAAVs.

In some embodiments, a “control” can refer to a subject or a tissue that contains human ACVR1^{R206H} proteins or the ACVR1 protein that comprises a single base mutation of guanine to adenine at position 206 of the sequence of the wild type ACVR1 while not being treated by the methods and compositions described in the present disclosure or any other methods.

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the therapeutic constructs in suitably formulated pharmaceutical compositions disclosed herein either subcutaneously, intraopancreatically, intranasally, parenterally, intravenously, intramuscularly, intradermally, intrathecally, femoral intramedullary, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used for delivery. In some embodiments, a preferred mode of administration is by portal vein injection. In some embodiments, a preferred mode of administration is by systemic injection, for example intravenous injection. In some embodiments, a preferred mode of administration is by intramuscular injection. In some embodiments, a preferred mode of administration is by intradermal injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery cither encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles ialso termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the compositions, nucleic acid sequences, isolated nucleic acids, or rAAVs may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for usc.

In addition to the methods of delivery described above, the following techniques are also contemplated as alternative methods of delivering the compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998). transdermal matrices (U.S. Pat. Nos. 5,770,219 and 5,783,208) and feedback-controlled delivery (U.S. Pat. No. 5,697,899).

In some aspects, disclosed herein are engineered T-cells for use in the treatment of FOP and/or its associated symptoms. As used herein, an “engineered T-cell” is a T lymphocyte that has been modified or engineered through genetic manipulation to enhance its therapeutic potential. In some embodiments, an engineered T cell comprises a chimeric antigen receptor (CAR). A “chimeric antigen receptor” or “CAR” as used herein refers to a recombinant cell receptor which directs the specificity or function of a cell (e.g., an immune cell) by providing both antigen-binding and cell-activating functions. Generally. CARs are fusion proteins comprising one or more antigen-binding domains (e.g., an extracellular domain), a transmembrane domain, and at least one cytoplasmic signaling domain (e.g., an intracellular domain), wherein the combination of extracellular domain, transmembrane domain and cytoplasmic signaling domain do not naturally occur together in nature. A cell expressing a CAR may produce, in some embodiments, an atypical cellular response (e.g., an increased, decreased, or different cellular response relative to a naturally-occurring cell).

A CAR may comprise one or more (e.g., 1, 2, 3, etc.) antigen-binding domains (e.g., an extracellular domain). As used herein, an “antigen-binding domain” refers to the domain of a protein or polypeptide external to the cellular membrane, wherein the domain's main function is to recognize (e.g., bind) and respond to a type of ligand (e.g., an antigen). An antigen-binding domain can be any domain that binds to the antigen including, but not limited to, monoclonal antibodies, single chain variable fragments (scFvs), polyclonal antibodies, synthetic antibodies, human antibodies, humanized antibodies, and fragments thereof. In some embodiments, the antigen-binding domain is derived from the same species in which the CAR will ultimately be used. For example, for usc in humans, in some embodiments, the antigen-binding domain of the CAR comprises a human antibody or fragment thereof.

In some embodiments, the antigen-binding domain comprises a human antibody or a fragment thereof. In some embodiments, the binding protein is an antibody, an antigen-binding portion of an antibody (e.g., a scFv), a ligand, a cytokine, or a receptor. In some embodiments, an antigen binding domain may comprise a site derived from a monoclonal antibody or a scFv. In some embodiments, the antigen-binding fragment is a scFv or a Fab fragment. In some embodiments, the antigen binding domain may bind an AAV capsid protein epitope. In some embodiments, the antigen binding domain targets an epitope of an AAV capsid protein having a serotype selected from AAV1, AAV2, AAV3, AAV3b, AAV4, AAV5, AAV6, AAV7, AAV8. AAVrh.8, AAV9, AAV10, AAVrh.10, AAVrh.39, AAVrh.43, AAV.PHPB, AAV.PHPB.e, AAVrh32.33, or a variant thereof. In some embodiments, the antigen-binding domain comprises a first portion comprising a monoclonal antibody or a scFv. In some embodiments, the antigen-binding domain is a monoclonal antibody (mAb).

In some embodiments, the mAb may be an mAb to, or targets, an epitope of an AAV capsid. In some embodiments, the mAb may be an mAb to, or targets, an epitope of an AAV capsid of serotype AAV1, AAV2, AAV3, AAV3b, AAV4, AAV5, AAV6, AAV7, AAV8, AAVrh.8, AAV9, AAV10, AAVrh.10. AAVrh.39, AAVrh.43, AAV.PHPB, AAV.PHPB.e, AAVrh32.33, or a variant thereof. In some embodiments, the mAb may be an mAb, or targets, an AAV9 capsid epitope, mAbs are antibodies that are made by immune cells (e.g., leukocytes) that are identical and bind to a shared epitope. Antibodies (e.g., immunoglobulins) are well known in the art, but generally are known to be large “Y” shaped proteins in the immune system which neutralize antigens. Each protein consists of two identical heavy chains and two identical light chains each having a variable (e.g., variable domain of heavy chain (VH) and variable domain of light chain (VL)). The variable domains contain complementarity determining regions, which specify the antigen the antibody will recognize (i.e. bind). In addition to the variable domains, the light chain comprises a constant domain (CL), and the heavy chain comprises three constant domains, which are numbered 1-3 as you move farther from the VH domain (e.g., CH1, CH2, CH3). Production of mAbs in all forms (e.g., humanized) are well known in the art and include traditional means such as animal based techniques. For example, the techniques generally entail: 1) exposing a subject (e.g., mouse, rabbit) to an antigen (e.g., capsid protein); 2) fusion into immortalized cell lines (e.g., myeloma cells); 3) cell culture and antibody screening; and 4) selection and recovery of cells presenting the mAb of interest) as well as other techniques such as phage display (generally: 1) the target proteins or nucleic acid sequences (e.g., capsid proteins) are immobilized to the wells of a microtiter plate; 2) a variety of nucleic acid sequences are expressed in a bacteriophage library in the form of fusions with the bacteriophage coat protein, so that they are displayed on the surface of the viral particle. The protein displayed corresponds to the genetic sequence within the phage; 3) this phage-display library is added to the microtiter plat allowing the phage time to bind, the dish is subsequently washed: 4) bound phage-displaying proteins remain attached to the dish, while all others are washed away; 5) attached phage may be enriched (through elution and culture, etc.) and steps 3 to 5 are optionally repeated one or more times, further enriching the phage library in binding proteins; 6) following further bacterial-based culture, the nucleic acids within in the interacting phage is sequenced to identify the interacting proteins or protein fragments). Any method of generating mAbs known in the art may be used to generate the CARs of the instant disclosure.

In some embodiments, a CAR-T cell is isolated from a subject having FOP. In some embodiments, a CAR-T cell is isolated from a subject having a mutation in an ACVR1 gene (e.g., an ACVR1^{R206H} mutation). In some embodiments, the CAR-T cell isolated from a subject with an ACVR1^{R206H} mutation has been genetically engineered to revert the R206H substitution to an Arginine at position 206 of the ACVR1^{R206H} protein. Methods of genetic engineering are well known in the art. Examples of genetic engineering techniques include, but are not limited to, CRISPR gene editing. Transcription Activator-like Effector Nucleases (TALENs), Zinc Finger Nucleases (ZFNs), engineered meganucleases, or re-engineered homing endonucleases.

In some embodiments, a CAR-T cell isolated from a subject having an ACVR1^{R206H} mutation is used for treatment of FOP in that same subject.

### Fibrodysplasia Ossificans Progressiva

Fibrodysplasia ossificans progressiva (FOP, #OMIM 135100) is an ultra-rare genetic disorder, characterized by progressive disabling heterotopic ossification (HO) of connective tissues, such as muscle, ligaments, tendons, fascia, and aponeuroses. HO is formed through endochondral ossification in childhood through adulthood, leading to immobility and severe pain (Pignolo et al., Orphanet J. Rare Dis. 2021, 16, 350). Flare-ups occur spontaneously in connective tissues or are triggered by injury, immunization via intramuscular injection, inflammation, or unknown reasons, followed by heterotopic bone formation. As a primary symptomatic management, high-dose corticosteroids for episodic flare-ups are used to decrease symptoms, including pain and edema (Pignolo et al., Pediatr. Endocrinol. Rev. 2013, 10 (Suppl. 2), 437-443), but there are no therapeutic options to suppressive the progressive HO in FOP.

Mutations in the activin A receptor, type 1 (ACVR1 or ALK2) cause FOP. Approximately 97% of identified patients with classic FOP have the same heterozygous single nucleotide change in ACVR1:c.617G>A; p.R206H (NCBI Reference Sequence NM_001111067.4:c.617G>A) (Pignolo et al., Orphanet J. Rare Dis. 2021, 16, 350), which lics within the glycine serine-rich domain of the ACVR1 protein. ACVR1 (encoded by the ACVR1 gene; NCBI Reference Sequence NG_008004.1) is a membrane-bound receptor of the BMP/TGFβ receptor family that canonically transduces signals of bone morphogenic protein (BMP) ligands. Under normal conditions, ACVR1 binds activin A, a member of the TGRβ superfamily, to initiate signaling through the SMAD2/3 pathway (Olsen et al., J. Cell Sci. 2018: 131 (11)). Treatment and prevention of FOP is challenging due to the difficulty of selectively inhibiting the aberrant activation of ACVR1^{R206H} signaling and the early onset of extra-skeletal formation. Currently, a retinoic acid receptor γ agonist (palovarotene capsules, Sohonos) was approved for treating FOP in Canada and USA but not in Europe, while an anti-Activin A antibody (REGN 2477), an immunosuppressant (rapamycin), and ACVR1 kinase inhibitors (IPN60130, Saractinib, INCB000928) are in clinical trials (Wentworth et al., Br. J. Clin. Pharmacol. 2019, 85, 1180-1187).

Recombinant adeno-associated viruses (rAAVs) are highly effective in transducing the liver, skeletal muscle, and the skeleton in vivo (Yang et al., Nat. Commun. 2019, 10, 2958), as well as the long-term expression of therapeutic gene (Herzog. R. W. Mol. Ther. 2020, 28, 341-341) and the robust safety profiles in both pre-clinical and clinical studies. Since gene therapy using rAAVs holds promise for treating many mono-genetic disorders, AAV gene therapy could be an attractive therapeutic approach for FOP in that approximately 97% of FOP patients harbor the recurrent missense ACVR1^{R206H} mutation (c.617G>A;p.R206H) (Shore et al., Nat. Genet. 2006, 38, 525-527). It was previously demonstrated that the AAV9 serotype is effective for the transduction of fibro-adipogenic progenitors (FAPs), major cells-of-origin of HO (Lees-Shepard et al., Nat. Commun. 2018, 9, 471; Wosczyna et al., J. Bone Miner. Res. 2012, 27, 1004-1017). Treatment with the AAV9 vector carrying ACVR1^{R206H}-specific silencer and codon-optimized human ACVR1 reduced the aberrant activation of bone morphogenetic protein (BMP)-Smad1/5 signaling and the chondrogenic/osteogenic differentiation of ACVR1^{R206H} skeletal progenitors. Accordingly, the systemic delivery of AAV gene therapy prevented spontaneous HO in FOP mice while trauma-induced HO was also decreased when administered transdermally to injured muscle (Yang et al., Nat. Commun. 2022, 13, 6175). However, since a high dose administration of rAAVs and/or AAV expression in non-HO tissues could potentially induce untoward immune responses and side effects in FOP patients, further vector improvement to reduce an injection dose by enhancing therapeutic efficacy and repress AAV expression in non-HO tissues is needed.

The ACVR1^{R206H} mutation has been reported to confer neoactivity to Activin A, which confers the aberrant activation of BMP-pSmad 1/5 signaling (Hino et al., Proc. Natl. Acad. Sci. USA 2015, 112, 15438-15443; Aykul et al., eLife 2020, 9, c54582), suggesting that Activin A functions as a gain-of-function ligand of the ACVR1^{R206H} receptor in FOP (Hatsell et al., Sci. Transl. Med. 2015, 7, 303ra137; Alessi Wolken et al., Bone 2018, 109, 210-217). Thus, in some aspects, provided herein are methods and compositions for treating FOP and its associated conditions using AAV vector that blocks the expression of the mutant ACVR1^{R206H} receptor and/or activin A. In some embodiments, mutant ACVR1^{R206H} is silenced and replaced with a wild-type or codon-optimized ACVR1 receptor. In some embodiments, the expression of AAV gene therapy is selectively repressed in the liver via the liver-abundant miR-122-mediated degradation.

FOP can lead to other ailments, such as alopecia, subcutaneous lipodystrophy, hearing loss, myelination effects, osteoarthritis, heightened inflammation, menstrual abnormalities and nephrolithiasis. In some embodiments, treatment of FOP includes treating one or more of these symptoms, or any other symptoms associated with FOP. In some embodiments, a subject (e.g., a human subject) experiencing FOP (or predisposed to developing FOP) has a nucleotide substitution (c.617G>A) that induces an amino acid substitution in the ACVR1 protein (p.R206H).

In some aspects, the disclosure provides compositions and methods for silencing Activin A (e.g., an INHBA allele). In some embodiments, the Activin A is a wild-type Activin A. In some embodiments, compositions and methods described herein comprise providing isolated nucleic acids that silence Activin A using a recombinant adeno-associated virus (rAAV) particle.

In some aspects, the disclosure provides compositions and methods for silencing a mutant allele of ACVR1 (e.g., ACVR1^{R206H}). In some embodiments, the mutant allele comprises at least one amino acid substitution. In some embodiments, compositions and methods described herein comprise providing isolated nucleic acids that silence a mutant allele of ACVR1 using a recombinant adeno-associated virus (rAAV) particle.

In some aspects, the present disclosure provides compositions and methods for providing a construct encoding a wild-type ACVR1 protein. In some aspects, the present disclosure provides compositions and methods for silencing a mutant allele of ACVR1 (e.g., ACVR1^{R206H}) and/or an INHBA allele and for providing a construct encoding a wild-type ACVR1 protein. In some embodiments, these compositions and methods comprise providing isolated nucleic acids using an rAAV delivery system.

### Therapeutic Methods

As disclosed herein, nucleic acids, rAAVs, and compositions described herein are useful for treating a subject having or suspected of having FOP and/or its associated symptoms. In some embodiments, aspects of the present disclosure provide methods of inhibiting heterotopic ossification in a subject. In some embodiments, aspects of the present disclosure provide methods of improving flare-up conditions in a subject having or suspected of having FOP. In some embodiments, aspects of the present disclosure provide methods of inhibiting Activin A and/or ACVR1^{R206H} expression in a cell.

As used herein, the term “treating” refers to the application or administration of a composition as described herein to a subject, who has a disease associated with heterotopic ossification (HO), such as FOP, a symptom of a disease associated with heterotopic ossification (HO), such as FOP, or a predisposition toward a disease associated with heterotopic ossification (HO), such as FOP (e.g., one or more mutations in a gene associated with FOP, such as AVCR1), with the purpose to cure, heal, alleviate, relieve, alter, remedy, ameliorate, improve, or affect the disorder, the symptom of the disease, or the predisposition toward the disease.

Alleviating a disease includes delaying the development or progression of the disease, or reducing disease severity. Alleviating the disease does not necessarily require curative results. As used therein, “delaying” the development of a disease (such as a disease associated with HO) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with heterotopic ossification (HO), such as FOP, includes initial onset and/or recurrence.

In some embodiments, the therapeutic methods as disclosed in this section comprise administering to a subject an isolated nucleic acid, a recombinant AAV (rAAV), a recombinant gene editing complex, or a vector, comprising a transgene as disclosed herein. A rAAV may comprise a modification that promotes its targeting to skeletal muscle or connective tissues. In some embodiments, the therapeutic methods as disclosed herein comprise administering to a subject a rAAV comprising a capsid protein and an isolated nucleic acid encoding an inhibitory nucleic acid. The rAAV may comprise an inhibitory nucleic acid (e.g., siRNA, shRNA, miRNA, or amiRNA). The inhibitory nucleic acid may decrease or increase expression of a target gene associated with FOP. In some embodiments, the rAAV or isolated nucleic acid comprises a transgene encoding an artificial microRNA that targets a gene associated with heterotopic ossification or the development of FOP. In some embodiments, the target gene is an Activin A gene (e.g., INHBA). In some embodiments, the target gene is a mutated AVCR1 (e.g., ACVR1^{R206H}). In some embodiments, the rAAV or isolated nucleic acid comprises a transgene encoding a wild-type ACVR1 protein. In some embodiments, the rAAV or isolated nucleic acid comprises a transgene encoding an Activin A trap (e.g., hACVR2A Kinase-del and/or hACVR2B Kinase-del).

In some aspects, the CAR-T cells of the disclosure are used as adjuvant therapies or with an adjuvant therapy. The term “adjuvant,” as may be used herein, refers to any therapy or treatment (e.g., composition, drug, or method based) which is used as an adjunct to the primary or initial therapy or treatment. Adjuvants may be administered concurrently (e.g., at the same time, simultaneously) with the primary or initial treatment or shortly after the administration of the primary or initial treatment. In some, but not all, cases an adjuvant modulates (e.g., increases, decreases) the effect of the primary or initial treatment. In some, but not all, cases an adjuvant is used to modulate (e.g., increase, decrease) a side effect of the primary or initial treatment. In some, but not all, cases an adjuvant is used to prepare (e.g., condition) a subject in anticipation of the primary or initial treatment or aid in the primary or initial treatment's effects or sustain or aid in the recovery of the subject after the primary or initial treatment.

In an aspect, the disclosure relates to a method comprising administering to a subject a recombinant T-regulatory cell comprising a CAR, such that the immune response of the subject to the AAV capsid protein is induced. In some embodiments a CAR-T cell is administered to a subject such that the immune response to an AAV capsid protein is inhibited. By administering a CAR-T cell recognizing an epitope of an AAV capsid protein, the immune response may be modulated by recognizing antigen binding sites on circulating immune cells (e.g. antigen-presenting cells) comprising the AAV epitope. It is believed immune cells expressing the AAV epitope will reduce in number, thus increasing or restoring baseline levels of APCs, and thus increasing the immune response to any contemporaneously or subsequently administered cell comprising an AAV capsid protein epitope. The duration of the reduced immunity state with respect to the targeted epitope may be temporary or long lasting, the time to administration of any subsequent therapy will be timed to exploit the reduced immunity state, which will be readily apparent to one of ordinary skill without undue experimentation.

Heterotopic ossification may be inhibited by between 50% and 99% (e.g., any integer between 50% and 99%, inclusive) using methods of the present disclosure. Flare-up conditions in a subject having or suspected of having FOP may be inhibited by between 50% and 99% (e.g., any integer between 50% and 99%, inclusive) using methods of the present disclosure. Without wishing to be bound by any theory, flare-up conditions refer to an exacerbation of a chronic disease such as FOP.

Exemplary embodiments of the invention will be described in more detail by the following examples. These embodiments are exemplary of the invention, which one skilled in the art will recognize is not limited to the exemplary embodiments.

### EXAMPLES

### Example 1

Activin A, a member of the TGF-β superfamily, was originally isolated from gonadal fluids and found to stimulate the secretion of pituitary follicle-stimulating hormone. TGF-βs play pivotal roles in regulation of tissue homeostasis, organ development, inflammation, cell proliferation, and apoptosis. In particular, Activin A has been recognized as a multifunctional cytokine expressed in a wide range of tissues and cells, and growing evidence implicates Activin A in the pathogenesis of a variety of disorders ranging from rheumatoid arthritis, bone disorders, sepsis, inflammatory bowel disease, atherosclerosis, and chronic heart failure to certain malignancies. Activin A systemic abundance rapidly increases during inflammation, and its overall roles are modulated at the tissue level by interactions with natural inhibitors, such as follistatin, and through the formation of diffusion gradients.

After the pro-proteins of Inhibins A (INHBA) are synthesized, the prodomains aid in folding and assembling of their mature peptides, by holding them in a dimerization-competent conformation, and results in homodimeric Activin A. Activin A, secreted from the cell, interacts extracellularly with specific components of the extracellular matrix, perlecan and agrin, by binding to their heparan sulfate side chains, thereby protecting Activin A from proteolysis, and perhaps presenting them at higher concentrations to their receptors. Binding of the mature dimers to their receptors displaces the prodomains. At the cellular level, one mechanism of activin A action involves its interaction with the cell surface type II kinase receptors ACVR2A and ACVR2B in association with the type I receptor ALK4 or ALK7 and signaling through the phosphorylation of the canonical effectors SMAD2 and SMAD3, which translocate into the nucleus and, in collaboration with transcriptional coactivators or corepressors (e.g., CBP/p300), regulating the expression of the target genes. In addition, Activin A may also activate SMAD-independent signaling pathway, such as the mitogen-activated protein kinases p38 and JNK.

Circulating levels of Activin A in the scrum of breast or prostate cancer patients with bone metastases are significantly higher compared with those of patients without bone metastases, indicating that targeting Activin A with ActRIIA-Fe may be useful for treating cancer-associated osteolytic lesions. In bisphosphonate-naïve multiple myeloma patients, anabolic improvements in bone mineral density and in bone formation were observed with ActRIIA-Fc, compared with placebo.

Finally, INHBA-deficient mice are born without whiskers, incisors, and mandibular molars. Approximately 30% also have cleft palate. They show neonatal lethality. Aberrant (absent) whisker development leads to secondary defects in whisker-associated trigeminal sensory function. Specific decrease in the number of retinal rod photoreceptors. These genetic studies suggest the importance of Activin A in development. Thus, Activin A inhibitors should be carefully designed to minimize side effects by improving target cell/tissue-specificity.

This example describes interfering RNAs (e.g., siRNAs, miRNAs, artificial miRNAs (amiRNAs, etc.) targeting nucleic acid sequences encoding Inhibin beta-A, which is a subunit of Activin protein, and expression vectors such as rAAV vectors (and rAAV particles), encoding such interfering RNAs. In some embodiments, the interfering nucleic acids and vectors are useful for treating bone disorders, for example heterotopic ossification (HO), Fibrodysplasia Ossificans Progressiva (FOP), and certain cancers associated with bone (e.g., bone cancers or cancers that have metastasized to bone tissue).

Briefly, rAAV vectors encoding a combination of (i) artificial miRNA (amiR) targeting INHBA. (ii) amiR targeting ACVR1^{R206H}, and (iii) a codon-optimized ACVR1 protein were developed to simultaneously suppress ACVR1^{R206H} and Activin A, while supplementing expression of wild-type ACVR1.

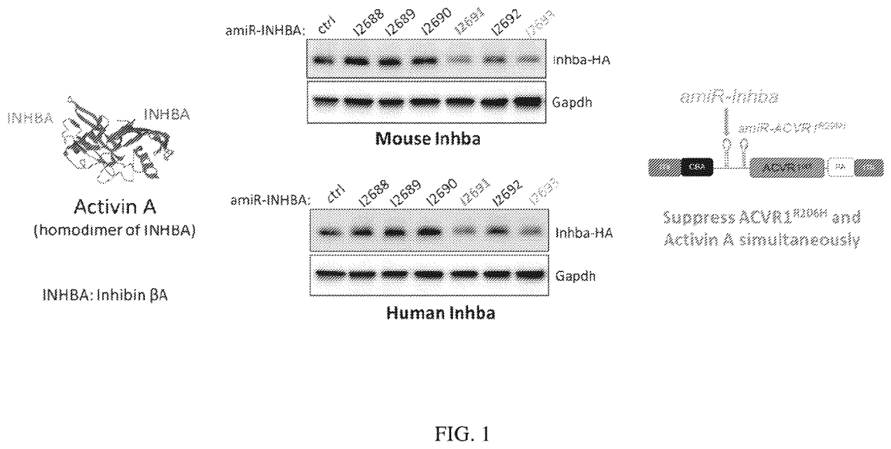

Artificial miRNAs targeting either mouse INHBA or human INHBA were tested (FIG. 1). HA-tagged mouse or human INHBA plasmid was co-transfected with an individual amiR-INHBA in HEK293 cells. Tested amiR-INHBA molecules were human INHBA amiRNA 1 (SEQ ID NO: 3; “I2688”), human INHBA amiRNA 2 (SEQ ID NO: 4; “I2689”), human INHBA amiRNA 3 (SEQ ID NO: 5; “I2690”), human INHBA amiRNA 4 (SEQ ID NO: 6; “I2691”), human INHBA amiRNA 5 (SEQ ID NO: 7; “I2692”), and human INHBA amiRNA 6 (SEQ ID NO: 8; “I2693”). These data indicate that several INHBA-targeting amiRs reduced expression of INHBA protein (FIG. 1).

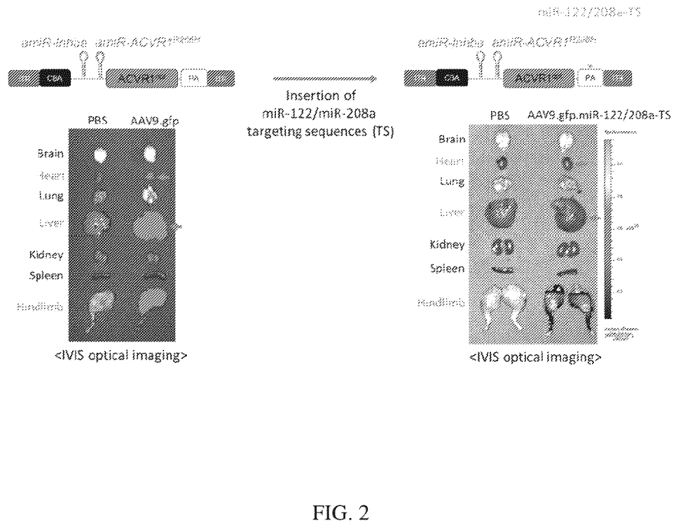

Vectors were further modified to include miRNA binding sites for miR-122 and miR-208a in order to de-target expression in liver cells and cardiac cells, respectively (FIG. 2). Vectors were packaged using AAV9 capsid protein and tested in vivo in mice. Data indicate that inclusion of the combination of miR-122 and miR-208a binding sites de-targeted the AAVs from heart and liver tissue (FIG. 2).

### Example 2

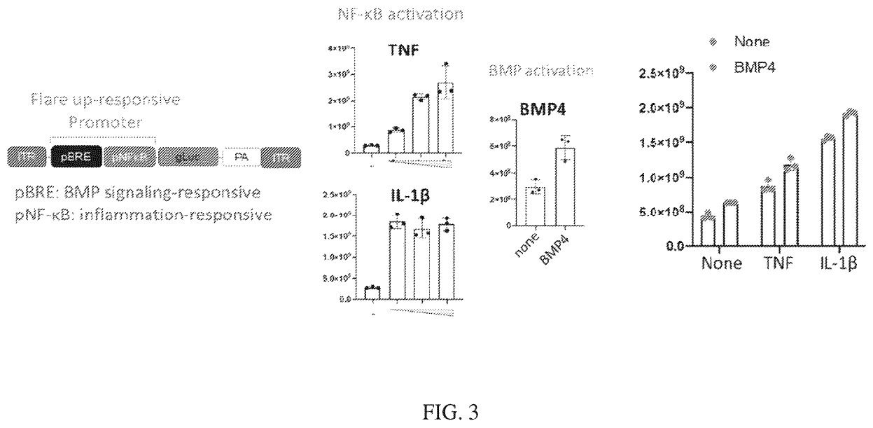

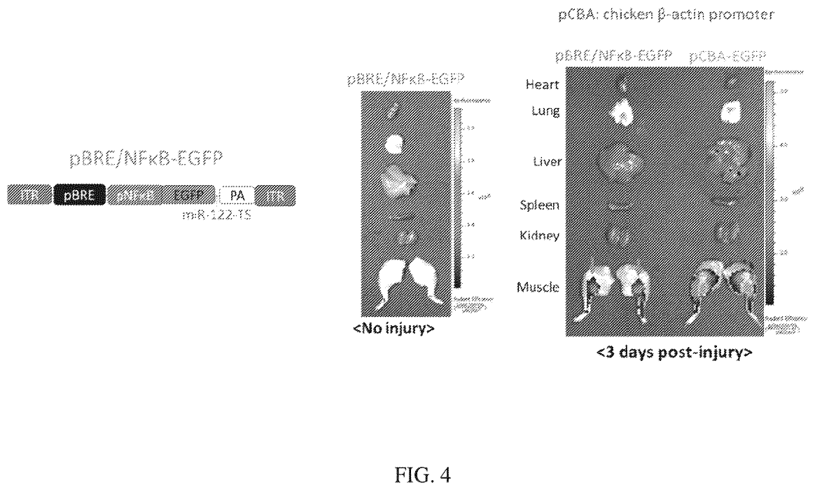

This example describes rAAV vectors comprising promoters that are responsive to inflammation in a subject (e.g., inflammation caused by a FOP flare-up). The rAAV vectors described in Example 1 above were further modified to swap out the chicken beta-actin (CB) promoter for a ‘flare-up-responsive promoter’ comprising (i) an inflammation-responsive promoter (pNF-κB) and (ii) a bone morphogenic protein (BMP) signaling-responsive promoter (pBRE) (FIGS. 3-4).

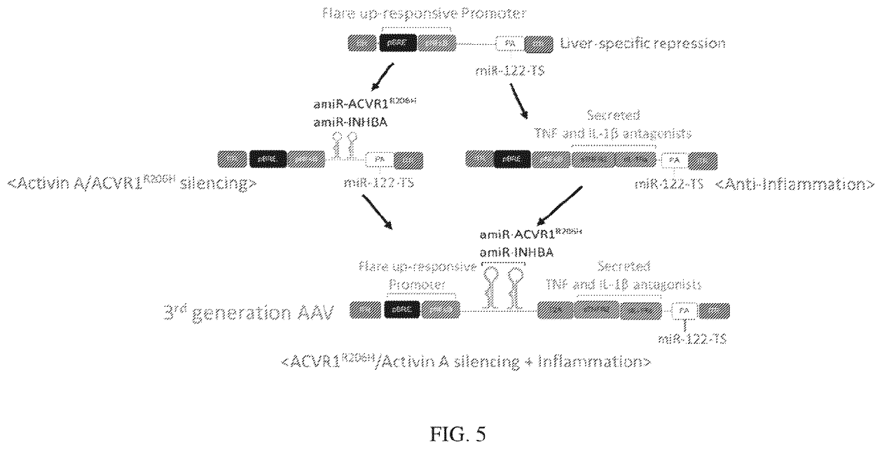

Additionally, rAAV vectors comprising (i) the flare-up-responsive promoter, (ii) artificial miRNA (amiR) targeting INHBA, (iii) amiR targeting ACVR1^{R206H}, and (iv) sequences encoding TNFα inhibitors and IL-1β inhibitors (e.g., soluble TNFR2 and soluble IL-1Rα, respectively) were developed to simultaneously suppress ACVR1^{R206H} and Activin A, while inhibiting inflammation. The designed ‘flare-up-responsive’ rAAVs also included miRNA binding sites for miR-122 for liver de-targeting (FIG. 5).

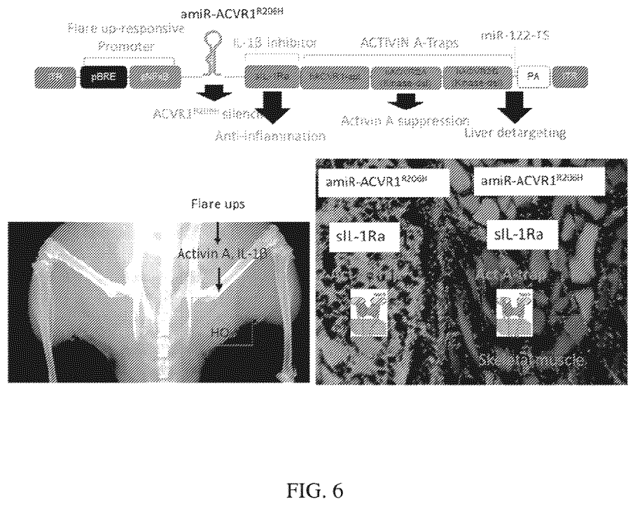

The ‘flare-up-responsive’ rAAVs may be further modified to include sequences encoding one or more of the following, cither as a substitute for or in addition to, the sTNFR2 and sIL1Rα sequences: ACVR2A having a kinase domain deletion (ACVR2A (kinase del)), and/or ACVR2B having a kinase domain deletion (ACVR2B (kinase del)), as shown in FIG. 6. In some embodiments, inclusion of the ACVR2A (kinase del) and/or ACVR2B (kinase del) functions as an Activin A ligand trap.

### Example 3

Fibrodysplasia ossificans progressiva (FOP) a rare disorder characterized by heterotopic ossification (HO). FOP develops by flare-ups that happen spontaneously or by minor trauma which result in HO, but also significant local inflammation and induction of inflammatory cytokines. However, patients have hyperinflammatory state even during periods without clinically evident HO, and have continually elevated cytokines including IL-3,7,8 and 10. Current treatments such as rapamycin and IL-1β inhibitors have been indicated as having some clinical efficacy suggesting immunosuppression could be therapeutic.

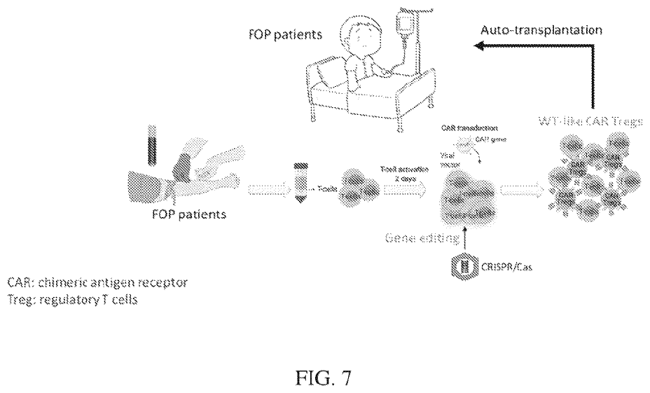

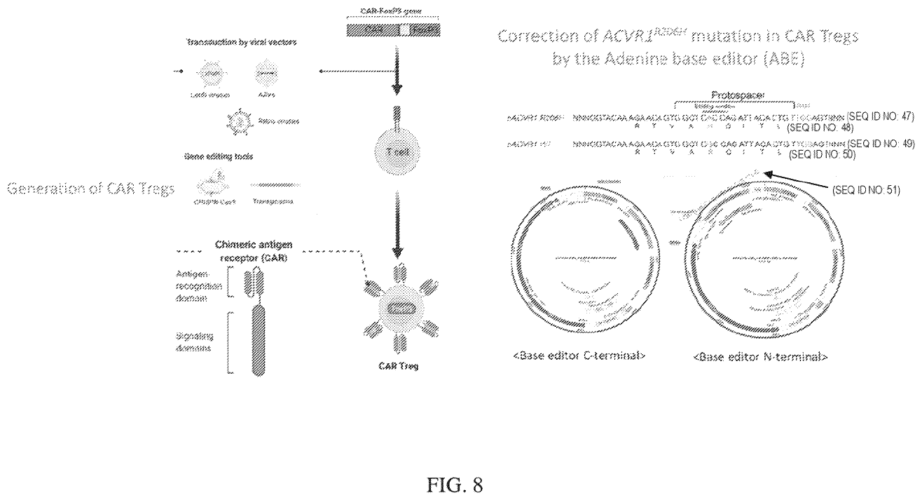

This example describes gene-editing approaches to treating Fibrodysplasia Ossificans Progressiva (FOP), and combination treatments comprising gene-edited cells and rAAV vectors. A CAR Treg that specifically recognizes AAV capsid, and therefore when used in combination with an AAV gene therapy can suppress local inflammation at the site of AAV transduced cells, was developed. The CAR-expressing cell is further modified by gene-editing to correct mutations associated with FOP. FIG. 7 shows a schematic depicting an immunotherapy protocol in which T-cells are isolated from a patient having FOP characterized by one or more mutations in the ACVR1 gene. The isolated T-cells are transduced with a chimeric antigen receptor (CAR)-encoding viral vector to transform the T cells into CAR-Treg cells. The CAR is designed to bind to AAV capsid antigens. The CAR-Treg cells are then subjected to gene editing using a base-editor in order to correct the ACVR1 mutation (e.g., an ACVR1^{R206H} mutation) in the CAR-Treg cells. The gene-edited, CAR-Treg cells are then injected back into the subject, where they suppress the host immune reaction to AAVs encoding INHBA amiRs (e.g., rAAV vectors described herein). FIG. 8 shows a schematic depicting a procedure for production of CAR-Treg cells that have been gene-edited to correct an ACVR1^{R206H} mutation.

### Example 4

### Up-Regulation of Activin a in HO-Tissues of FOP Mice

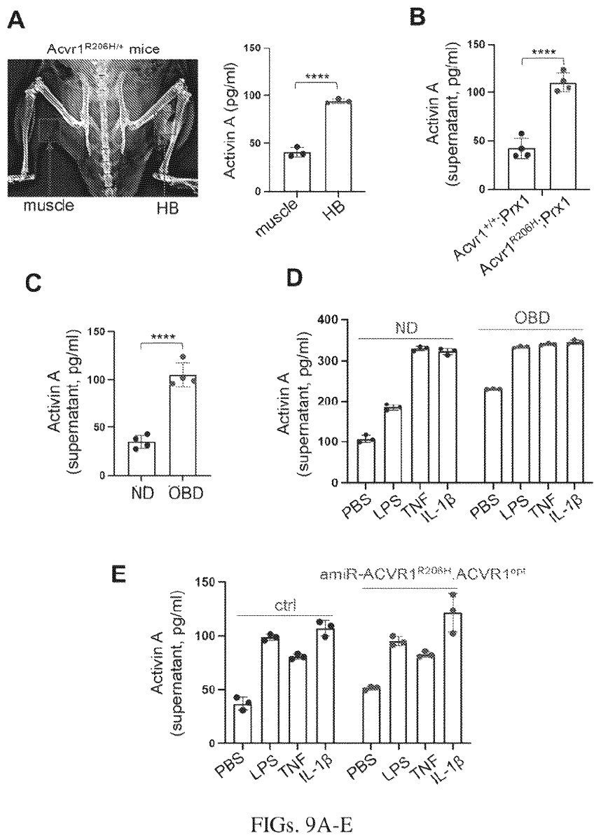

Activin A is a member of the TGF-β superfamily and plays pivotal roles in the regulation of tissue homeostasis, organ development, inflammation, cell proliferation, and apoptosis (Namwanje et al., Cold Spring Harb. Perspect. Biol. 2016, 8). Activin A expression is rapidly upregulated by inflammatory macrophages and other activated immune cells, which leads to the production of pro-inflammatory cytokines, such as TNF, IL-1β, and IL-6, and the recruitment of mast cells, thereby initiating HO pathogenesis (Convente et al., J Bone Miner. Res. 2018, 33. 269-282). Using single cell RNA-seq analysis and developmental trajectories, it has been demonstrated that in addition to inflammatory immune cells. Sox9⁺ mesenchymal and chondrogenic progenitors also expressed Activin A in BMP2-induced HO tissues (Mundy et al., Sci. Signal. 2021, 14), consistent with the finding that Activin A expression was markedly increased in established HO tissues within the injured muscles of ACVR1^{R206H/+} mice compared to non-HO tissues in contralateral legs (FIG. 9A). Likewise, elevated levels of Activin A were also detected in bone marrow-derived stromal cells (BMSCs) isolated from FOP mice (ACVR1^{R206H};Prx1) relative to WT mice (ACVR1^{+/+};Prx1, FIG. 9B), indicating that heterotopic bone-forming, chondrogenic/osteogenic cells are also Activin A-producing cell populations in FOP. Notably, human BMSCs were well responsive to osteogenic cues (FIG. 9C) or pro-inflammatory stimuli, including lipopolysaccharide (LPS), tumor necrosis factor (TNF), or interleukin 1β (IL-1β, FIGS. 9D-9E), resulting in the upregulation of Activin A. These results suggested that chondrogenic/osteogenic cells may facilitate HO development by producing Activin A under flare-up conditions.

### Generation of AAV Gene Therapy Targeting Activin A and ACVR1^{R206H}

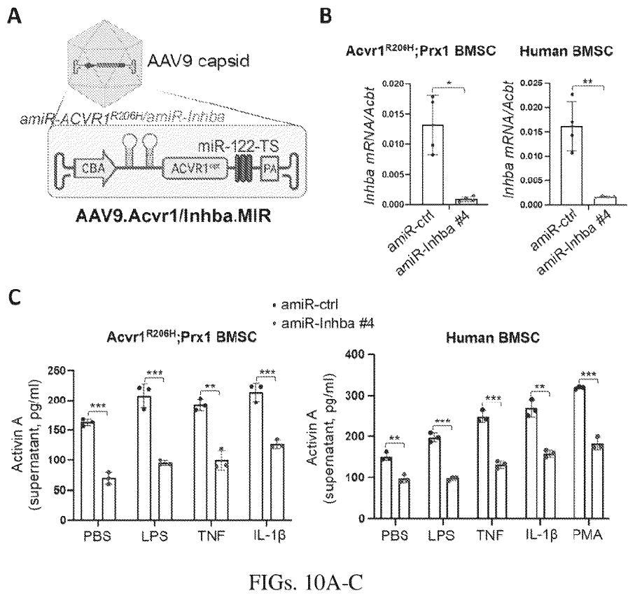

To examine whether upregulation of Activin A in ACVR1^{R206H};Prx1 BMSCs by pro-inflammatory stimuli could be reversed by replacing the mutant ACVR1^{R206H} receptor with wildtype (WT) ACVR1 receptor, ACVR1^{R206H};Prx1 BMSCs were transduced with an AAV vector carrying artificial miRNA targeting ACVR1^{R206H} mRNA (amiR-ACVR1^{R206H}; SEQ ID NO: 35) and codon-optimized human ACVR1 (ACVR1^{opt}; SEQ ID NO: 28) (Yang et al. Nat. Commun. 2022, 13, 6175), then stimulated with LPS, TNF, or IL-1β. ELISA analysis showed little to no alteration in Activin A expression levels in AAV-treated cells (FIG. 9E), suggesting that expression of ACVR1^{R206H} receptor in BMSCs is dispensable to the upregulation of Activin A. Thus, an AAV vector was generated to silence expression of both Activin A and ACVR1^{R206H} receptor to inhibit the Activin A-ACVR1^{R206H} signaling pathway more effectively. Given that Activin A is a homodimer of Inhibins βA (INHBA) (Namwanje et al., Cold Spring Harb. Perspect. Biol. 2016, 8), to silence Activin A expression, six artificial miRNAs (amiRs #1-#6 (SEQ ID NOs: 3-8) comprising the nucleic acid sequence of SEQ ID NOs: 41-45) 20 targeting shared sequences of mouse and human INHBA were designed and cloned into the AAV vector genome that contains amiR ACVR1^{R206H} 1 and ACVR1^{opt} (FIG. 10A). HA tagged mouse or human INHBA plasmid was co-transfected with amiR-INHBA in HEK293 cells, demonstrating amiR-INHBA #4 and #6 to be potent silencers of mouse and human INHBA expression. Note that amiR-INHBA #1-#6 correspond to the amiR-INHBA constructs tested in FIG. 1. In particular, the AAV9 carrying amiR-INHBA #4 was highly effective for silencing both mouse and human INHBA expression in ACVR1^{R206H};Prx1 BMSCs and human BMSCs, respectively (FIG. 10B, hereafter referred to as amiR-INHBA). These cells also showed a significant decrease in upregulation of Activin A by pro-inflammatory stimuli or the mitogen phorbol myristate acetate (PMA. FIG. 10C). Thus, an AAV vector genome simultaneously silencing the expression of Activin A and ACVR1^{R206H}, and expressing ACVR1opt receptor, was successfully generated and packaged into AAV9 capsid (hereafter referred to AAV9.ACVR1/INHBA).

Generation of AAV Gene Therapy with Liver-Specific Repression

Previous studies demonstrated that a single dose (5×10¹³ vg/kg) of systemically delivered AAV9 transduced liver, heart, skeletal muscle, and bone in adult mice (Yang et al., Nat. Commun. 2019, 10, 2958) while skeletal muscle was transduced when transdermally (t.d.) administered at 5×10¹² vg/kg (Yang et al., Nat. Commun. 2022, 13, 6175). Because FOP patients show little to no clinical manifestations in the liver (Pignolo et al., Pediatr. Endocrinol. Rev. 2013, 10 Suppl 2, 437-448), an AAV gene vector for FOP gene therapy was designed to repress the AAV's expression in the liver in order to improve the safety of the AAV gene therapy. Given that the silencing of transgene expression in liver exploited the natural expression of the abundant (>60,000 copies/cell) miRNA, miR-122, which is present in hepatocytes of virtually all animals (Chang et al., RNA Biol. 2004, 1, 106-113; Lagos-Quintana ct al., Curr. Biol. 2002, 12, 735-739), endogenous complementary sites for miR-122 (miR-122-TS) were inserted into the 3′ untranslated region (UTR) of a gfp or ACVR1^{opt} transgene in the AAV vector genome and packaged into AAV9 capsid (AAV9.gfp.MIR. AAV9.ACVR1/INHBA.MIR (comprising ACVR1^{opt} and INHBA amiRNA #4). IVIS optical imaging system revealed that t.d. injection of AAV9.gfp.MIR successfully expressed GFP in the skeletal muscle with little to no expression detected in the brain, heart, lung, liver, kidney, or spleen. This was consistent with fluorescence microscopy showing GFP expression in the skeletal muscle but not in brain, heart, or liver. Thus, the local delivery of the AAV9 gene therapy with liver-abundant miR-122-mediated repression enabled the de-targeting of non-skeletal organs except for skeletal muscle.

AAV Gene Therapy Inhibits Aberrant ACVR1^{R206H} Signaling and Osteogenic Differentiation of ACVR1^{R206H} Skeletal Progenitors

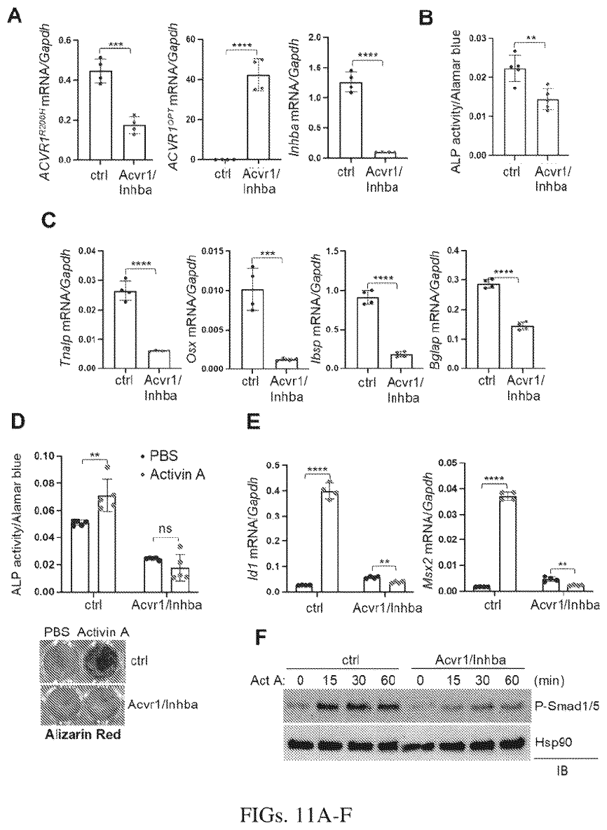

To examine in vitro therapeutic efficacy of AAV9 carrying amiR-ACVR1^{R206H} amiR-INHBA.ACVR1^{opt}.MIR (ACVR1/INHBA), ACVR1^{R206H} 1;Prx1 BMSCs were transduced by the AAV vectors and six days later, knockdown efficiency of INHBA or ACVR1^{R206H} and expression of ACVR1^{opt} were confirmed by RT-PCR analysis (FIG. 11A). Osteogenic differentiation of AAV-transduced BMSCs were examined, demonstrating a significant decrease in alkaline phosphatase (ALP) activity (FIG. 11B), along with the expression of osteogenic genes, including Tnalp, Osterix (Osx), Bone sialoprotein (Ibsp), and Osteocalcin (Bglap, FIG. 11C). Additionally, Activin A-induced ALP activity and extracellular mineralization of these cells were substantially reduced by treatment with ACVR1/INHBA relative to control (FIG. 11D). This was accompanied with a significant decrease in the induction of BMP-responsive genes, Id1 and Max2 (FIG. 11E), and phosphorylation of BMP signaling mediators, Smad1 and 5, in response to Activin A (FIG. 11F). It was thus shown that the AAV gene therapy of silencing INHBA and ACVR1^{R206H} expression and expressing the ACVR1^{opt} receptor is a potent inhibitor of Activin A-induced aberrant BMP signaling and osteogenic differentiation in FOP skeletal progenitors.

### AAV Gene Therapy Prevents Trauma-Induced HO in FOP Mice

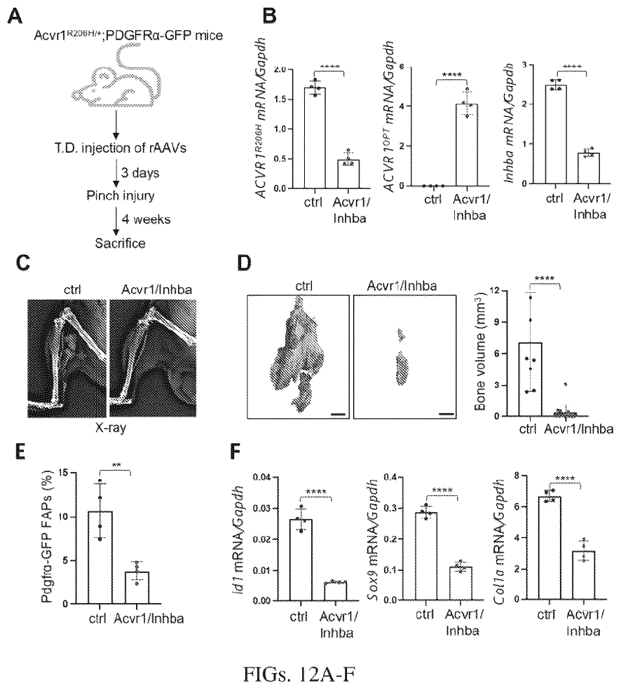

Since constitutive expression of human ACVR1^{R206H} allele causes perinatal lethality in mice (Kaplan et al., Dis. Model Mech. 2012, 5, 756-739; Chakkalakal et al., J. Bone Miner. Res. 2012, 27, 1746-1756), mice harboring a conditional knock-in allele of human ACVR1^{R206H} (ACVR1^{(R206H)Fl}) were crossed with Sox2-Cre mice (ACVR1^{(R206H)Fl/+};Sox2-cre, hereafter referred to as ACVR1^{R206H/+}) where expression of Cre recombinase in epiblasts mediates expression of ACVR1^{R206H} in all tissues. Heterozygous mice (ACVR1^{R206H/+}) were further crossed with Pdgfrα-GFP reporter mice to label Pdgfrα-expressing FAPs, the major cells-of-origin of HO (ACVR1^{R206H/+};Pdgfrα·GFP) (Lees-Shepard et al., Nat. Commun. 2018, 9, 471; Wosczyna et al., J. Bone Miner. Res. 2012, 27, 1004-1017). To examine the ability of AAV gene therapy to prevent trauma-induced HO in FOP. 8-week-old ACVR1^{R206H/+};Pdgfrα-GFP mice were treated with a single dose of transdermal (t.d.) injection of AAV9.ctrl.MIR or AAV9.ACVR1/INHBA.MIR (comprising ACVR1^{opt} and INHBA amiRNA #4) and three days later, pinch injury was introduced into the gastrocnemius muscle (FIG. 12A). Four weeks after the injury, Pdgfrα-expressing FAPs were isolated from the treated muscles by FACS sorting using GFP⁺Scal⁺CD31⁻CD45⁻ markers and knockdown efficiency of ACVR1^{R206H} or INHBA, or expression of ACVR1^{opt} in these cells was validated using RT-PCR analysis (FIG. 12B). Radiography (FIG. 12C) and microCT (FIG. 12D) analyses demonstrated that heterotopic bone mass in the injured muscle was substantially decreased when treated with ACVR1/INHBA.MIR relative to ctrl.MIR. Thus, local delivery of AAV gene therapy targeting both Activin A and ACVR1R206H receptor is highly effective for the prevention of trauma-induced HO in the skeletal muscle of ACVR1^{R206H/+} mice.

To visualize how ACVR1/INHBA.MIR expression prevents the pathogenesis of HO in FOP mice, pinch injury was introduced in the gastrocnemius muscle of AAV-treated ACVR1^{R206H/+};Pdgfrα-GFP mice and four weeks later, Pdgfrα-expressing FAPs in the injured muscle were monitored by fluorescence microscopy using GFP expression. As expected, GFP-expressing Pdgfrα⁺ FAPs within forming HO lesions in ctrl. MIR-treated muscle were highly proliferative and a subset of the cells differentiated into heterotopic bone-forming osteoblasts. This was consistent with histologic analysis showing heterotopic bone and chondrogenic anlagen in the skeletal muscle of these mice. By contrast, there was little to no evidence of HO lesions in the skeletal muscle expressing ACVR1/INHBA.MIR, where GFP-expressing Pdgfrα⁺ FAPs were primarily present in muscle interstitium, similar to Pdgfrα⁺ FAPs present in wildtype muscle (Yang et al., Nat. Commun. 2022, 13, 6175). Likewise, flow cytometry analysis demonstrated a significant decrease in the numbers of GFP⁺Scal⁺CD31⁻CD45⁻ FAPs in the skeletal muscle treated with ACVR1/INHBA.MIR relative to ctrl.MIR (FIG. 12c). Notably, these cells showed a significant reduction in Id1 (BMP-responsive gene), Sox9 (chondrogenic gene), and Col1α1 (osteogenic gene) expression (FIG. 12F), suggesting that local delivery of AAV9.ACVR1/INHBA.MIR (comprising ACVR1^{opt} and INHBA amiRNA #4) effectively suppresses ACVR1^{R206H}-induced aberrant BMP signaling and resultant chondrogenesis and osteogenesis of Pdgfrα⁺ FAPs in ACVR1^{R206H/+} mice. Thus, inhibition of Activin A and ACVR1^{R206H} receptor in the skeletal muscle of FOP mice using nucleic acids targeting INHBA and ACVR1^{R206H} receptor is a promising approach to inhibit the initiation process of traumatic HO in FOP.

### Treatment of Traumatic HO in FOP Mice by AAV Gene Therapy

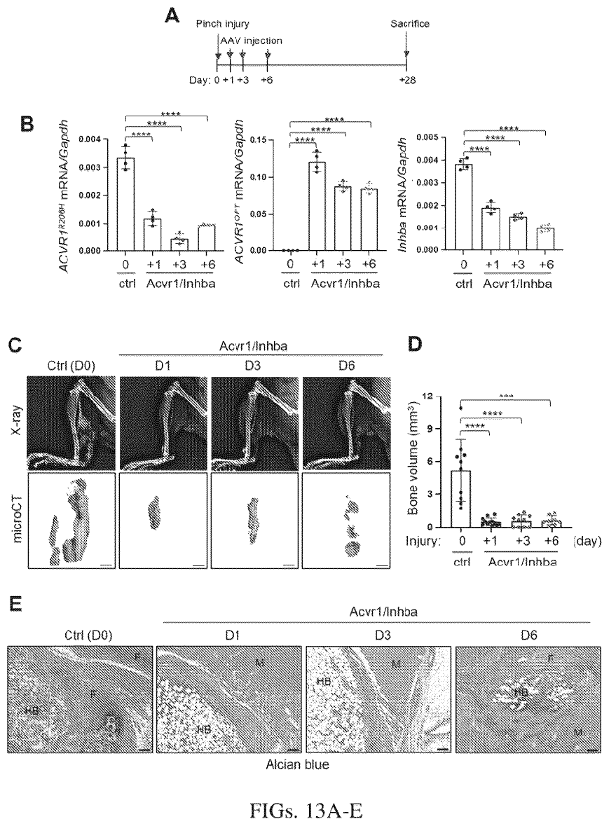

It has been demonstrated that upon muscle injury, FOP mice undergo HO lesion progression from immune cell infiltration (Days 1-3), muscle degeneration and fibroproliferative response (Days 3-7), chondrogenesis (Days 7-14), through osteogenesis with heterotopic bone marrow establishment (Days 14-28) (Lounev et al., J. Bone Joint Surg. Am. 2009, 97, 652-663; Kaplan et al., J. Bone Joint Surg. Am. 2007, 89, 347-357). To define the stage at which AAV gene therapy can suppress HO progression, pinch injury was introduced into the gastrocnemius muscle of 3 week old ACVR1^{R206H/+} mice and one, three, or six days later, AAV9 carrying ctrl.MIR or ACVR1/INHBA.MIR were t.d. injected (FIG. 13A). Four weeks later, knockdown efficiency of ACVR1^{R206H} or INHBA and expression of ACVR1^{opt} in treated muscles was validated using RT-PCR analysis (FIG. 13B). Radiography, microCT, and histo-pathological evaluation of AAV-treated muscle was performed, demonstrating that heterotopic bone mass was markedly decreased by local delivery of ACVR1/INHBA.MIR at different time points (FIGS. 13C-13E). As expected, control-treated muscle developed heterotopic bone with bone marrow compartment via endochondral pathway including formation of fibrotic tissue and chondrogenic analgen. However, treatment with ACVR1/INHBA.MIR on day 1 or 3 post-injury resulted in a significant decrease in the volume of heterotopic bone, which were filled with adipose tissue (inside) and surrounded by regenerated muscle (outside). When treated with ACVR1/INHBA.MIR on day 6 post-injury, small-sized heterotopic bones with bone marrow compartment (inside) were formed and surrounded by regenerated muscle (outside. FIG. 13E). These results demonstrate that treatment with AAV gene therapy before the chondrogenic stage effectively suppresses HO progression. Taken together, it was shown that an AAV-mediated combination gene therapy that executes silencing of INHBA and ACVR1^{R206H} and expresses ACVR1^{opt} is a promising approach to suppress disabling HO, providing proof-of-concept for clinical translation to FOP patients.

### Discussion:

The data provided herein demonstrate that AAV gene therapy targeting Activin A and its receptor ACVR1^{R206H} is a potent suppressor of HO in FOP. In some embodiments, provided herein is a multi-functional AAV vector which executes both silencing of Activin A and ACVR1^{R206H} and expression of codon optimized human ACVR1 (ACVR1^{opt}), while simultaneously de-targeting the liver. As a key stimulator of HO. Activin A is upregulated during chondrogenic/osteogenic differentiation of FOP skeletal progenitors in HO tissues. The AAV gene therapy provided in this example downregulated production of Activin A in these cells. Thus, AAV treatment ablated aberrant activation of BMP-Smad1/5 signaling and osteogenic differentiation of ACVR1^{R206H/+} skeletal progenitors. Additionally, it was highly effective for prevention of trauma-induced HO in ACVR1^{R206H/+} mice. Finally, the insertion of complementary sequences of liver abundant miR-122 repressed the AAV's expression in the liver with no FOP pathology. Thus, in some embodiments, the AAV gene therapies provided herein are used as therapeutic strategies to suppress HO in FOP patients who have lifelong progression of HO.

Currently FDA-approved AAV gene therapies for lipoprotein lipase deficiency (Glybera) (Carpentier et al., The Journal of Clinical Endocrinology and Metabolism 2012, 97, 1635-1644), inherited retinal dystrophy (Luxturna) (Chiu et al., Interational Journal of Molecular Sciences 2021, 22), spinal muscular atrophy (Zolgensma) (Mendell et al., JAM Neurology 2021, 78, 834-841), Duchenne muscular dystrophy (Elevidys) (Mullard, A. Nat. Rev. Drug Discov. 2023. doi: 10.1038/d41573-023-00103-y), and hemophila B (Hemgenix) (Naddaf, M. Nature 2022, 612, 388-389), utilize gene replacement strategies that introduce genes encoding missing proteins or encoding corrective proteins. Conversely, since autosomal dominant, heterozygous mutations in ACVR1^{R206H} (c.617G>A;p.R206H) cause FOP (Shore. E. M. and Kaplan, F. S. Bone 2008, 43, 427-433; Shore et al., Nat. Genet. 2006, 38, 525-527), the gene therapy approaches provided herein, in some embodiments, were designed to replace the mutant ACVR1^{R206H} receptor with the healthy ACVR1 receptor via the combination approach of: 1) silencing of the expression of the ACVR1^{R206H} receptor at the mRNA level using ACVR1^{R206H} allele-specific amiR and 2) dilution of aberrant BMP-Smad1/5 signaling by replacing the mutant ACVR1^{R206H} receptor with a codon-optimized wild-type ACVR1 receptor. In other embodiments, the therapeutic efficacy of the gene therapy approaches herein was further enhanced by silencing expression of the key HO inducer Activin A at the mRNA level using amiR targeting INHBA. These results provide proof-of-concept demonstration that AAV-mediated inhibition of both Activin A and its receptor. ACVR1^{R206H}, in injured muscle is highly effective in suppressing the initiation and progression of HO in FOP mice.

### Methods

### Mice

Mice were housed in standard cages in a temperature-controlled room (22-24° C.) with a 12 h dark-light cycle and fed with a standard chow (LabDiet, #5P7622:22.5% protein, 5.4% fat, 4% fiber, 50% polysaccharide). Mice harboring a knock-in allele of human ACVR1^{R206H} (ACVR1^{(R206H)Fl}) (Lyu et al., Biomedicines 2021, 9. doi: 10.3390/biomedicines9060630) were kindly gifted from the International FOP Association via Dr. Daniel Perrien (Emory University) and maintained on C57BL/6J background. Wildtype mouse exons 5-10, neomycin-resistant gene (neo cassette), one F3 site, and one loxP site were deleted by Cre recombinase, resulting in expression of human eDNA exons 6-11 harboring the R206H mutation and eGFP. ACVR1^{(R206H)Fl} mice were crossed with Sox2-Cre mice (ACVR1^{(R206H)Fl/+};Sox2-cre, hereafter referred to ACVR1^{R206H/+}) where expression of Cre recombinase in epiblasts mediates expression of ACVR1^{R206H} in all tissues; Prx1-Cre mice (ACVR1^{R206H)Fl};Prx1-cre) where expression of Cre recombinase in Prx1+ skeletal progenitors in the limb mesenchyme mediates ACVR1^{R206H}-driven HO. Sox2-Cre and Prx1-Cre mice were purchased from Jackson Laboratory and maintained on C57BL/6J background. To label Pdgfrα expressing FAPs, ACVR1^{R206H/+} mice were crossed with Pdgfrα-GFP reporter mice (Jackson Laboratory. C57BL/6J). Mouse genotypes were determined by PCR using tail genomic DNA. No sex-specific differences in HO phenotypes were observed in these mice. Primer sequences are available upon request. Control littermates were used and analyzed in all experiments. All animals were used in accordance with the NIH Guide for the Care and Use of Laboratory Animals and were handled according to protocols approved by the University of Massachusetts Chan Medical School Institutional Animal Care and Usc Committee (IACUC).

### Cell Culture and Reagents

Human bone marrow-derived mesenchymal stromal cells (BMSCs, #7500) were purchased from ScienCell Research Laboratories. They were cultured according to the manufacturer's manuals. Mouse FAPs were FACS sorted from the digested skeletal muscle of 4-week-old ACVR1^{R206H/+};PDGFRα-GFP mice using cell surface markers (PDGFRα⁻GFP⁺Scal⁺CD31⁻CD45⁻) and cultured in DMEM (Corning) containing 20% FBS (Corning), and 1% penicillin/streptomycin (Corning) (Lees-Shepard et al., Nat. Commun. 2018, 9, 471). Mouse bone marrow-derived stromal cells (BMSCs) were isolated from the long bones of 4-week-old ACVR1^{(R206H)Fl};Prrx1-Cre mice. Cells were maintained in α-MEM medium (Gibco) containing 10% FBS (Corning), 2 mM L-glutamine (Corning), 1% penicillin/streptomycin (Corning), and 1% nonessential amino acids (Corning) while they were differentiated into mature osteoblasts under osteogenic medium containing ascorbic acid (200 uM, Sigma, #A8960) and β-glycerophosphate (10 mM, Sigma, #G9422). Recombinant lipopolysaccharide (#LPS-B5), TNF (#210-TA), IL-1β/IL-IF2 (#201-LB), and Activin A (#338-AC) proteins were purchased from InvivoGen and R&D systems, respectively. Plasmids expressing HA-tagged human or mouse INHBA were purchased from Sino Biological.

rAAV Vector Design and Production

pAAVsc-CB6-ACVR1^{opt} was generated by replacing the mCherry reporter with a codon-optimized version of the human ACVR1 complementary DNA (ACVR1^{opt}), and then, the chicken β-actin (CBA) intron in the plasmid was replaced with the MassBiologics (MBL) intron to reduce the AAV vector genome size. The artificial miRNAs (amiR) against human ACVR1^{R206H} or mouse/human INHBA were designed using a custom Excel macro, which considers miR-33 scaffold design rules to generate optimized amiR cassettes (Xie et al., Mol. Ther. 2020, 28, 422-430). Plasmids were constructed by Gibson assembly and standard molecular biology methods. DNA sequences for amiR-33-ctrl, amiR-33-human ACVR1^{R206H} amiR-33-INHBA were synthesized as gBlocks and cloned into the intronic region of the pAAVse CB6 mCherry plasmid at the restriction enzyme sites (PstI and BglII) (Lin et al., Nat. Commun. 2022, 13, 6869). After verified by sequencing, the constructs were packaged into AAV9 capsid. rAAV production was performed by transient transfection of HEK293 cells, purified by CsCl sedimentation, titered by droplet digital PCR (ddPCR) on a QX200 ddPCR system (Bio-Rad) using the Egfp or mCherry prime/probe set (Gao. G. and Sena-Esteves, M. Molecular Cloning 2012, 2, 1209-1313).

### Quantitative RT-PCR, Immunoblotting, and ELISA Analyses

Total RNA was purified from cells and tissues using QIAzol (QIAGEN) and cDNA was synthesized using the High-Capacity cDNA Reverse Transcription Kit (Applied Biosystems, #4368814). Quantitative RT-PCR was performed using iTaq™ Universal SYBR® Green Supermix (Bio-Rad, #1725122) with CFX connect RT-PCR detection system (Bio-Rad). To measure mRNA levels of the indicated genes in the injured areas or HO lesions of AAV-treated mice, the tibialis muscles were snap-frozen in liquid nitrogen for 30 sec and in turn homogenized in 1 ml of QIAzol for 1 min.

Cells were lysed in TNT lysis buffer (150 mM NaCl, 1% Triton X-100, 1 mM EDTA, 1 mM EGTA, 50 mM NaF, 1 mM Na, VO₄, 1 mM PMSF and protease inhibitor cocktail (Sigma)) and protein amounts from cell lysates were measured using DC protein assay (BioRad). Equivalent amounts of proteins were subjected to SDS-PAGE, transferred to Immobilon-P membranes (Millipore), immunoblotted with anti-GAPDH antibody (1:1000, EMD Millipore, #CB1001), anti-phospho-Smad1/5 antibody (1:1000, Cell Signaling Technology, #9516), and anti-HSP90 antibody (1:1000, BioLegend, #675402), and developed with ECL (ThermoFisher Scientific). Immunoblotting with anti-HSP90 antibody or anti-GAPDH antibody was used as a loading control.

AAV-treated muscles were lysed using NP-40 lysis buffer (ThermoScientfic, #J60766) and protein levels of Activin A in the lysates were measured using the Activin A quantikine ELISA kit (R&D systems, #DAC00B). Alternatively, the supernatant was harvested from AAV-treated ACVR1R206H BMSCs and then subjected to the Activin A ELISA kit.

### Trauma-Induced HO

Prevention model: 5×10¹² vg/kg of rAAV9.gfp.miR.122.TS or rAAV9.amiR-INHBA/ACVR1^{R206H}.ACVR1^{opt}.miR-122.TS was injected transdermally (t.d.) into the tibial muscle of 3-week-old ACVR1^{R206H/+} mice. Three days later, pinch injury was employed into the gastrocnemius muscle. mRNA levels of INHBA, ACVR1^{R206H}, and ACVR1^{opt} were assessed by RT-PCR four weeks post-injury. Heterotopic bone mass in the muscle was assessed by radiography and microCT four weeks post-injury. Alternatively, pinch injury was performed on the gastrocnemius muscle of AAV treated ACVR1^{R206H/+};Pdgfrα GFP mice and four weeks later, AAV-transduced FAPs were FACS sorted from HO sites using GFP⁺Scal⁺CD31⁻CD45⁻ markers. GFP-expressing FAPs in the cryosectioned muscle were visualized using fluorescence microscopy.

Treatment model: 8-week-old ACVR1^{R206H/+} tibial muscles were injected t.d. with rAAV9.gfp.miR-122.TS or rAAV9.amiR-INHBA/ACVR1^{R206H}.ACVR1^{opt}.miR-122.TS one, three, or six days after pinch injury. Four weeks later, mice were euthanized for HO assessment.

### Osteoblast Differentiation

To assess extracellular matrix mineralization in AAV-treated osteoblasts, cells were washed twice with IX phosphate-buffered saline (PBS) and fixed in 70% EtOH for 15 min at room temperature. Fixed cells were washed twice with distilled water and then stained with 2% Alizarin red solution (Sigma. #A5533) for 5 min. Cells were then washed three times with distilled water and examined for the presence of calcium deposits. Mineralization was quantified by the acetic acid extraction method (Gregory et al., Anal. Biochem. 2004, 329, 77-84). For alkaline phosphatase (ALP) staining, osteoblasts were fixed with 10% neutral buffered formalin and stained with the solution containing Fast Blue (Sigma. #FBS25) and Naphthol AS-MX (Sigma, #855). Alternatively, osteoblasts were incubated with 10-fold diluted Alamar Blue solution (Invitrogen, #DAL1100) for cell proliferation. Subsequently, cells were washed and incubated with a solution containing 6.5 mM Na₂CO₃, 18.5 mM NaHCO₃, 2 mM MgCl₂, and phosphatase substrate (Sigma, #S0942), and ALP activity was measured by a spectrometer (BioRad).

### MicroCT and Radiography

MicroCT (uCT35; SCANCO Medical AG; Bruttisellen, Switzerland) was used for qualitative and quantitative assessment of heterotopic bone in muscle and performed by an investigator blinded to the genotypes of the animals under analysis. MicroCT scanning was conducted at 55 kVp and 114 mA energy intensity with 300-ms integration time. The specific voxel size used for whole tibia was 12 μm. All images were reconstructed using image matrices of 1024×1024 pixels. For heterotopic bone evaluation, whole tibia muscle area was contoured. 3D reconstruction images were obtained from contoured 2D images by methods based on distance transformation of the binarized images. Alternatively, the Inveon multimodality 3D visualization program was used to generate fused 3D viewing of multiple static or dynamic volumes of microCT modalities (Siemens Medical Solutions USA, Inc). All images presented are representative of the respective genotypes (n>5).

Trident Specimen Radiography system (Hologic. USA) was used to generate detailed radiographic images of the whole mouse body after euthanasia. The X ray beam intensity was 1 mA 28 kv˜30 KV with AEC (automatic exposure control) for fast image acquisition.

### Histology

For histological analysis, whole hindlimb with muscle was dissected from AAV-treated mice, fixed in 10% neutral buffered formalin for two days at room temperature. and decalcified by 14% EDTA tetrasodium salt, pH 7.6 for 3-4 weeks at 4° C. Samples were kept in 70% ethanol until processed on a vacuum infiltration tissue processor. Sections were done on a microtome (HistoCore Multicut; Leica, USA) at a thickness of 6 μm along the coronal plate from anterior to posterior. Slides were stained with hematoxylin and cosin (H&E) or alcian blue/hematoxylin/orange G.

For frozen sectioning, dissected specimens were fixed in 4% paraformaldehyde for 2˜3 days, followed by 15 days of semi-decalcification using 14% EDTA tetrasodium salt, pH 7.6 at 4° C. Infiltration was processed using 20% sucrose solution prior to OCT embedding. Slides were prepared on Cryostat (LM3050s; Leica USA) at a thickness of 12 μm. Slides were stained with DAPI and hematoxylin and cosin (H&E).

### Statistical Methods

All data were presented as the mean±SD. Sample sizes were calculated on the assumption that a 30% difference in the parameters measured would be considered biologically significant with an estimate of sigma of 10-20% of the expected mean. Alpha and Beta were set to the standard values of 0.05 and 0.8, respectively. No animals or samples were excluded from analysis and animals were randomized to treatment versus control groups, where applicable. For relevant data analysis, where relevant, the the Shapiro-Wilk normality test was first performed for checking normal distributions of the groups. If normality tests passed, two-tailed, unpaired Student's t-test was used and if normality tests failed, and Mann-Whitney tests were used for the comparisons between two groups. For the comparisons of three or four groups, one-way ANOVA was used if normality tests passed, followed by Tukey's multiple comparison test for all pairs of groups. If normality tests failed, the Kruskal-Wallis test was performed and was followed by Dunn's multiple com-parison test. GraphPad PRISM software (v6.0a. La Jolla, CA) was used for statistical analysis. P<0.05 was considered statistically significant. *, P<0.05; **, P<0.01; ***, P<0.001; and ****, P<0.0001.

### Additional Embodiments

Additional embodiments of the present disclosure are encompassed by the following numbered paragraphs:

1. An isolated nucleic acid comprising a transgene comprising a nucleic acid sequence encoding one or more artificial microRNAs (amiR) targeting an INHBA RNA transcript, flanked by adeno-associated virus (AAV) inverted terminal repeats (ITRs).

2. The isolated nucleic acid of paragraph 1, wherein the transgene further comprises a promoter operably linked to the nucleic acid sequence encoding the amiR.

3. The isolated nucleic acid of paragraph 2, wherein the promoter is a chicken beta actin (CBA) promoter or a flare-up-responsive promoter.

7. The isolated nucleic acid of paragraph 3, wherein the flare-up-responsive promoter comprises a first portion comprising a NF-κB promoter, and a second portion comprising a bone morphogenic protein (BMP) signaling-responsive promoter (pBRE).

5. The isolated nucleic acid of any one of paragraphs 1 to 4, wherein the transgene further comprises one or more amiRs targeting ACVR1 gene, optionally wherein the one or more amiRs targeting an ACVR1^{R206H} allele, optionally wherein the one or more amiRs targeting an ACVR1^{R206H} allele comprise the sequence set forth in SEQ ID NO: 35.

6. The isolated nucleic acid of any one of paragraphs 1 to 5, wherein the one or more amiRs targeting an INHBA RNA transcript comprise the sequence set forth in any one of SEQ ID NOs: 3-8.

7 The isolated nucleic acid of any one of paragraphs 1 to 6, wherein the transgene further comprises a nucleic acid sequence encoding an ACVR1 protein, a nucleic acid sequence encoding a soluble TNFR2 (sTNFR2) protein, a nucleic acid sequence encoding a soluble IL-1Rα (sIL-1Rα) protein, or a combination thereof.

8. The isolated nucleic acid of paragraph 7, wherein the nucleic acid sequence encoding the ACVR1 protein is codon-optimized.

9. The isolated nucleic acid of any one of paragraphs 1 to 8, wherein the transgene further comprises one or more miRNA binding sites, optionally wherein the one or more miRNA binding sites comprise one or more miR-122 binding sites, one or more miR-208a binding sites, or a combination thereof.

10. The isolated nucleic acid of any one of paragraphs 1 to 9, wherein the AAV ITRs are AAV2 ITRs.

11. A vector comprising the isolated nucleic acid of any one of paragraphs 1 to 10, wherein the vector is a plasmid.

12. A recombinant adeno-associated virus (rAAV) comprising:
(i) the isolated nucleic acid of any one of paragraphs 1 to 10; and
(ii) one or more AAV capsid proteins.

13. The rAAV of paragraph 12, wherein the capsid protein has a tropism for muscle or bone.

14. The rAAV of paragraph 12 or 13, wherein the AAV capsid protein is of a serotype selected from AAV1, AAV2, AAV3, AAV4, AAV5, AAV6, AAV7, AAV8, AAV9, AAV10, or a variant thereof.

15. An isolated nucleic acid comprising a transgene flanked by adeno-associated virus (AAV) inverted terminal repeats (I′TRs), wherein the transgene comprises
(i) a flare-up-responsive promoter comprising a first portion comprising a NF-κB promoter, and a second portion comprising a bone morphogenic protein (BMP) signaling-responsive promoter (pBRE);
(ii) a nucleic acid sequence encoding one or more artificial microRNAs (amiR) targeting an INHBA RNA transcript;
(iii) a nucleic acid sequence encoding one or more artificial microRNAs (amiR) targeting an ACVR1^{R206H} RNA transcript;
(iv) a nucleic acid sequence encoding a soluble IL-1Rα (sIL-1Rα) protein;
(v) a codon-optimized nucleic acid sequence encoding a wild-type ACVR1 protein;
(vi) a nucleic acid sequence encoding an Activin A trap comprising an ACVR2A kinase deletion mutant protein and an ACVR2B kinase deletion mutant protein; and
(vii) one or more miRNA binding sites.

16. The isolated nucleic acid of paragraph 16, wherein the one or more miRNA binding sites comprise one or more miR-122 binding sites, one or more miR-208a binding sites, or a combination thereof.

17. The isolated nucleic acid of paragraph 15 or 16, wherein the AAV ITRs are AAV2 ITRs.

18. A vector comprising the isolated nucleic acid of any one of paragraphs 15 to 17, wherein the vector is a plasmid.

19. An rAAV comprising:
(i) the isolated nucleic acid of any one of paragraphs 15 to 17; and
(ii) one or more AAV capsid proteins.

20. The rAAV of paragraph 19, wherein the capsid protein has a tropism for muscle or bone.

21. The rAAV of paragraph 19 or 20, wherein the AAV capsid protein is of a serotype selected from AAV1, AAV2, AAV3, AAV4, AAV5, AAV6, AAV7, AAV8, AAV9, AAV10, or a variant thereof.

22. An engineered T cell comprising a chimeric antigen receptor (CAR-T), wherein the T cell has been obtained from a subject having an R206H mutation in an ACVR1 gene, and the cell has been engineered by base-editing to revert the R206H mutation in the ACVR1 gene.

23. The engineered T cell of paragraph 22, wherein the CAR binds to one or more AAV capsid antigens.

24. A method for treating a disease or disorder associated with bone in a subject in need thereof, the method comprising administering the isolated nucleic acid, rAAV, and/or engineered T cell of any of the preceding paragraphs to the subject.

25. The method of paragraph 24, wherein the subject is a human.

26. The method of paragraph 24 or 25, wherein the administration comprises administration to the muscle of the subject, bone of the subject, or systemic administration.

27. An isolated nucleic acid comprising the sequence set forth in any one of SEQ ID NOs: 3-8, 21, and 24.

28. The isolated nucleic acid of paragraph 27, further comprising a miR-33 backbone.

29. An rAAV vector comprising the sequence set forth in any one of SEQ ID NOs: 9-20, 22, and 23, with the proviso that the rAAV vector does not comprise an EGFP or luciferase protein coding sequence.

30. An rAAV comprising the isolated nucleic acid of paragraph 27 or 28 and at least one AAV capsid protein.

31. A composition comprising:
(a) a first nucleic acid sequence encoding an inhibitory nucleic acid that targets an INHBA transcript; and
(b) a second nucleic acid sequence encoding a wild-type Activin A Receptor, type 1 (ACVR1) protein.

32. A composition comprising:
(a) a first nucleic acid sequence encoding an inhibitory nucleic acid that targets an INHBA transcript;
(b) a second nucleic acid sequence encoding a wild-type Activin A Receptor, type 1 (ACVR1) protein; and
(c) a third nucleic acid sequence encoding an inhibitory nucleic acid that targets a mutant ACVR1 transcript.

33. The composition of paragraph 32, wherein the mutant ACVR1 transcript is a ACVR1^{R206H} transcript.

34. The composition of any one of paragraphs 31-33, wherein the inhibitory nucleic acid that targets an INHBA transcript is a double-stranded RNA (dsRNA), small interfering RNA (siRNA), short hairpin RNA (shRNA), microRNA (miRNA), or artificial microRNA (amiR) that targets an INHBA transcript.

35. The composition of any one of paragraphs 31-34, wherein the inhibitory nucleic acid that targets an INHBA transcript comprises a nucleic acid sequence having at least 90%, at least 95%, at least 97.5%, at least 99%, or 100% identity to any one of SEQ ID NOS: 41-45.

36. The composition of paragraph 35, wherein the amiR that targets an INHBA transcript comprises a nucleic acid sequence having at least 90%, at least 95%, at least 97.5%, at least 99%, or 100% identity to any one of SEQ ID NOs: 3-8.

37. The composition of any one of paragraphs 34-36, wherein the amiR that targets an INHBA transcript comprises a nucleic acid sequence having at least 70%, at least 80% at least 90%, at least 95%, at least 97.5%, or at least 99% identity to SEQ ID NO: 6.

38. The composition of any one of paragraphs 32-37, wherein the inhibitory nucleic acid that targets a mutant ACVR1 transcript is a double-stranded RNA (dsRNA), small interfering RNA (siRNA), short hairpin RNA (shRNA), microRNA (miRNA), or artificial microRNA (amiR) that targets a mutant ACVR1 transcript.

39. The composition of any one of paragraphs 32-38, wherein the inhibitory nucleic acid that targets a mutant ACVR1 transcript comprises a nucleic acid sequence having at least 90%, at least 95%, at least 97.5%, at least 99%, or 100% identity to SEQ ID NO: 46.

40. The composition of paragraph 38 or 39, wherein the amiR that targets a mutant ACVR1 transcript comprises the sequence set forth in SEQ ID NO: 35.

41. The composition of any one of paragraphs 31-40 further comprising a promoter operably linked to the first nucleic acid sequence, the second nucleic acid sequence, and/or the third nucleic acid sequence.

42. The composition of paragraph 41, wherein the promoter is a chicken beta actin (CBA) promoter.

43. The composition of paragraph 41, wherein the promoter is a flare-up-responsive promoter.

42. The composition of paragraph 41, wherein the flare-up-responsive promoter comprises a first portion comprising a NF-κB promoter, and a second portion comprising a bone morphogenic protein (BMP) signaling-responsive promoter (pBRE).

43. The composition of any one of paragraphs 31-42, wherein the nucleic acid sequence encoding the ACVR1 protein is codon-optimized.

44. The composition of any one of paragraphs 31-43, wherein the nucleic acid sequence encoding the ACVR1 protein comprises a nucleic acid sequence having at least 90%, at least 95%, at least 97.5%, or at least 99% identity to SEQ ID NO: 30.

45. The composition of any one of paragraphs 31-44, wherein the first nucleic acid sequence, the second nucleic acid sequence, and/or the third nucleic acid sequence further comprises one or more miRNA binding sites, optionally wherein the one or more miRNA binding sites are de-targeting miRNA binding sites.

46. The composition of paragraph 45, wherein the one or more miRNA binding sites comprise one or more miR-122 binding sites, one or more miR-208a binding sites, or a combination thereof,
optionally wherein the one or more miR-122 binding sites comprise or consist of the nucleic acid sequence of SEQ ID NO: 33 and/or wherein the one or more miR-208a binding sites comprise or consist of the nucleic acid sequence of SEQ ID NO: 34.

47. The composition of any one of paragraphs 31-46, wherein the ACVR1 protein comprises an amino acid sequence having at least 90%, at least 95%, at least 97.5%, at least 99%, or 100% identity to SEQ ID NO: 25.

48. The composition of any one of paragraphs 31-47, wherein the first nucleic acid sequence, the second nucleic acid sequence, and/or the third nucleic acid sequence are encoded within a single nucleic acid.

49. The composition of paragraph 48, wherein the single nucleic acid further comprises one or more adeno-associated virus (AAV) inverted terminal repeats (ITRs).

50. A vector comprising the composition of paragraph 48 or 49.

51. A recombinant adeno-associated (rAAV) comprising:
(a) the composition of any one of paragraphs 31-49; and
(b) an AAV capsid protein.

52. The rAAV of paragraph 51, wherein the rAAV targets skeletal tissue or bone.

53. The rAAV of paragraphs 51 or 52, wherein the capsid protein is an AAV1, AAV6, AAV7, AAV8, AAV9, AAV-DJ/8, AAV-Rh10, AAV-retro, AAV-PHP.B, AAV8-PHP.eB, or AAV-PHP.S capsid protein.

54. A pharmaceutical composition comprising (i) the composition of any one of paragraphs 31-49, the vector of paragraph 50, the rAAV of any one of paragraphs 51-53 and (ii) a pharmaceutically acceptable excipient.

55. The pharmaceutical composition of paragraph 54, wherein the composition is formulatec for injection, optionally wherein the injection is transdermal (t.d.) injection.

56. A method for preventing or reducing heterotopic ossification (HO) in a subject comprising administering to the subject an effective amount of the isolated nucleic acid, vector, rAAV, engineered T cell, composition, or pharmaceutical composition of any one of the preceding paragraphs.

57. A method for treating fibrodysplasia ossificans progressiva (FOP) in a subject comprising administering to the subject an effective amount of the isolated nucleic acid, vector, rAAV, engineered T cell, composition, or pharmaceutical composition of any one of the preceding paragraphs.

58. A method for reducing BMP-Smad1/5 signaling in a subject comprising administering to the subject an effective amount of the isolated nucleic acid, vector, rAAV, engineered T cell, composition, or pharmaceutical composition of any one of the preceding paragraphs.

59. The method of any one of paragraphs 56-58, wherein the subject is a human, optionally wherein the subject has at least one copy of a ACVR1^{R206H} allele.

REPRESENTATIVE SEQUENCES

>Mousc INHBA mRNA targct sequence (SEQ ID NO: 1)
ATGCCCTTGCTTTGGCTGAGAGGATTTCTGTTGGCAAGTTGCTGGATTATAGTGAGGAGTTCCCC
CACCCCAGGATCCGAGGGGCACGGCTCAGCCCCGGACTGCCCGTCCTGTGCGCTGGCCACCCTTC
CGAAGGATGGACCTAACTCTCAGCCAGAGATGGTAGAGGCTGTCAAGAAGCACATCTTAAACAT
GCTGCACTTGAAGAAGAGACCCGATGTCACCCAGCCGGTGCCCAAGGCGGCGCTTCTCAACGCG
ATCAGAAAGCTTCATGTGGGTAAAGTGGGGGAGAACGGGTATGTGGAGATAGAGGACGACATT
GGCAGGAGGGCCGAAATGAATGAACTCATGGAGCAGACCTCGGAGATCATCACCTTTGCCGAGT
CAGGCACAGCCAGGAAGACACTGCACTTTGAGATTTCCAAGGAAGGCAGTGACCTGTCAGTAGT
GGAGCGTGCAGAAGTGTGGCTCTTCCTGAAAGTCCCCAAGGCTAACAGAACCAGGACCAAAGTC
ACCATCCGTCTATTTCAGCAGCAGAAGCACCCACAGGGCAGCTTGGACACGGGGGATGAGGCCG
AGGAAATGGGCTTAAAGGGGGAGAGGAGTGAACTGTTGCTATCAGAGAAAGTAGTTGATGCTCG
GAAGAGTACCTGGCACATCTTTCCAGTGTCCAGCAGCATCCAGCGCCTGCTGGACCAGGGAAAG
AGTTCCCTGGACGTGCGGATTGCTTGTGAGCAGTGCCAGGAGAGTGGTGCCAGTCTAGTGCTTCT
GGGCAAGAAGAAGAAGAAAGAGGTGGATGGAGATGGGAAGAAGAAAGATGGGAGTGACGGAG
GGCTGGAAGAGGAAAAGGAACAGTCACATAGACCTTTCCTCATGCTGCAGGCTAGGCAGTCCGA
AGACCACCCTCATCGCAGGCGTAGGCGGGGCTTGGAGTGCGACGGCAAGGTCAACATTTGCTGT
AAGAAACAGTTCTTTGTCAGCTTCAAGGACATTGGCTGGAATGACTGGATCATTGCTCCCTCTGG
CTATCACGCCAATTATTGTGAGGGGGAGTGCCCAAGCCACATAGCAGGCACCTCTGGGTCCTCG
CTCTCCTTCCACTCAACAGTCATTAACCACTACCGCATGAGGGGTCACAGCCCCTTTGCCAACCT
TAAGTCATGCTGTGTGCCCACCAAGCTGAGACCCATGTCCATGCTGTATTACGATGATGGTCAAA
ACATCATCAAAAAGGACATTCAAAACATGATTGTGGAGGAGTGTGGCTGCTCCTAG
>Human INHBA mRNA targct sequence (SEQ ID NO: 2)
ATGCCCTTGCTTTGGCTGAGAGGATTTCTGTTGGCAAGTTGCTGGATTATAGTGAGGAGTTCCCC
CACCCCAGGATCCGAGGGGCACAGCGCGGCCCCCGACTGTCCGTCCTGTGCGCTGGCCGCCCTC
CCAAAGGATGTACCCAACTCTCAGCCAGAGATGGTGGAGGCCGTCAAGAAGCACATTTTAAACA
TGCTGCACTTGAAGAAGAGACCCGATGTCACCCAGCCGGTACCCAAGGCGGCGCTTCTGAACGC
GATCAGAAAGCTTCATGTGGGCAAAGTCGGGGAGAACGGGTATGTGGAGATAGAGGATGACAT
TGGAAGGAGGGCAGAAATGAATGAACTTATGGAGCAGACCTCGGAGATCATCACGTTTGCCGAG
TCAGGAACAGCCAGGAAGACGCTGCACTTCGAGATTTCCAAGGAAGGCAGTGACCTGTCAGTGG
TGGAGCGTGCAGAAGTCTGGCTCTTCCTAAAAGTCCCCAAGGCCAACAGGACCAGGACCAAAGT
CACCATCCGCCTCTTCCAGCAGCAGAAGCACCCGCAGGGCAGCTTGGACACAGGGGAAGAGGCC
GAGGAAGTGGGCTTAAAGGGGGAGAGGAGTGAACTGTTGCTCTCTGAAAAAGTAGTAGACGCTC
GGAAGAGCACCTGGCATGTCTTCCCTGTCTCCAGCAGCATCCAGCGGTTGCTGGACCAGGGCAA
GAGCTCCCTGGACGTTCGGATTGCCTGTGAGCAGTGCCAGGAGAGTGGCGCCAGCTTGGTTCTCC
TGGGCAAGAAGAAGAAGAAAGAAGAGGAGGGGGAAGGGAAAAAGAAGGGCGGAGGTGAAGGT
GGGGCAGGAGCAGATGAGGAAAAGGAGCAGTCGCACAGACCTTTCCTCATGCTGCAGGCCCGG
CAGTCTGAAGACCACCCTCATCGCCGGCGTCGGCGGGGCTTGGAGTGTGATGGCAAGGTCAACA
TCTGCTGTAAGAAACAGTTCTTTGTCAGTTTCAAGGACATCGGCTGGAATGACTGGATCATTGCT
CCCTCTGGCTATCATGCCAACTACTGCGAGGGTGAGTGCCCGAGCCATATAGCAGGCACGTCCG
GGTCCTCACTGTCCTTCCACTCAACAGTCATCAACCACTACCGCATGCGGGGCCATAGCCCCTTT
GCCAACCTCAAATCGTGCTGTGTGCCCACCAAGCTGAGACCCATGTCCATGTTGTACTATGATGA
TGGTCAAAACATCATCAAAAAGGACATTCAGAACATGATCGTGGAGGAGTGTGGGTGCTCATAG
>human INHBA amiRNA I (SEQ ID NO: 3)
GGCAGCCTTGGAGTGGGTTCCTGCCCCCTCGGGCACACAAACAGAGCTGAAGACCACCCTGGGC
ACCTCCTTGGCTGGCCGCATACCTCCTGGCGGGCAGCTGTGtctttcttcttcttcttgcccTGTTCTGGTGGTA
CCCAGGGCAAGATCAGGAAGAAAGACACAGAGGCCTGCCTGGCCCTCGAGAGACTGCCCTGACT
GAAGGCCCTATCAGGTGGGGGAGGGGATCCTGATAGAGGGCACTGCTGCCACTGTTGGGGCCCA
AG
>human INHBA amiRNA 2 (SEQ ID NO: 4)
GGCAGCCTTGGAGTGGGTTCCTGCCCCCTCGGGCACACAAACAGAGCTGAAGACCACCCTGGGC
ACCTCCTTGGCTGGCCGCATACCTCCTGGCGGGCAGCTGTGtttgccactgtcttctctggaTGTTCTGGTGGT
ACCCATCCAGAGATCATAGTGGCAAACACAGAGGCCTGCCTGGCCCTCGAGAGACTGCCCTGAC
TGAAGGCCCTATCAGGTGGGGGAGGGGATCCTGATAGAGGGCACTGCTGCCACTGTTGGGGCCC
AAG
>human INHBA amiRNA 3 (SEQ ID NO: 5)
GGCAGCCTTGGAGTGGGTTCCTGCCCCCTCGGGCACACAAACAGAGCTGAAGACCACCCTGGGC
ACCTCCTTGGCTGGCCGCATACCTCCTGGCGGGCAGCTGTGttcctttcaagtatctctcctTGTTCTGGTGGTA
CCCAAGGAGAGAATCCTGAAAGGAACACAGAGGCCTGCCTGGCCCTCGAGAGACTGCCCTGACT
GAAGGCCCTATCAGGTGGGGGAGGGGATCCTGATAGAGGGCACTGCTGCCACTGTTGGGGCCCA
AG
>human INHBA amiRNA 4 (SEQ ID NO: 6)
GGCAGCCTTGGAGTGGGTTCCTGCCCCCTCGGGCACACAAACAGAGCTGAAGACCACCCTGGGC
ACCTCCTTGGCTGGCCGCATACCTCCTGGCGGGCAGCTGTGtgatctccgaggtctgctccaTGTTCTGGTGGT
ACCCATGGAGCAGTGCCCGGAGATCACACAGAGGCCTGCCTGGCCCTCGAGAGACTGCCCTGAC
TGAAGGCCCTATCAGGTGGGGGAGGGGATCCTGATAGAGGGCACTGCTGCCACTGTTGGGGCCC
AAG
>human INHBA amiRNA 5 (SEQ ID NO: 7)
GGCAGCCTTGGAGTGGGTTCCTGCCCCCTCGGGCACACAAACAGAGCTGAAGACCACCCTGGGC
ACCTCCTTGGCTGGCCGCATACCTCCTGGCGGGCAGCTGTGttttgatgatgttttgaccatTGTTCTGGTGGTA
CCCAATGGTCAATTCGTCATCAAAACACAGAGGCCTGCCTGGCCCTCGAGAGACTGCCCTGACT
GAAGGCCCTATCAGGTGGGGGAGGGGATCCTGATAGAGGGCACTGCTGCCACTGTTGGGGCCCA
AG
>human INHBA amiRNA 6 (SEQ ID NO: 8)
GGCAGCCTTGGAGTGGGTTCCTGCCCCCTCGGGCACACAAACAGAGCTGAAGACCACCCTGGGC
ACCTCCTTGGCTGGCCGCATACCTCCTGGCGGGCAGCTGTGtctttcttcttcttcttgcccTGTTCTGGTGGTA
CCCAGGGCAAGATCAGGAAGAAAGACACAGAGGCCTGCCTGGCCCTCGAGAGACTGCCCTGACT
GAAGGCCCTATCAGGTGGGGGAGGGGATCCTGATAGAGGGCACTGCTGCCACTGTTGGGGCCCA
AG
>human INHBA amiRNA 1-EGFP rAAV (SEQ ID NO: 9)
ctgcgcgctcgctcgctcactgaggccgcccgggcaaagcccgggcgtcgggcgacctttggtcgcccggcctcagtgagcgagcgagcgcgcagagag
ggagtgtagccatgctctaggaagatcaattcggtacaattcacgcgtcgacattgattattgactctggtcgttacataacttacggtaaatggcccgcctggctg
accgcccaacgaccccgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatgcccagtacatgacctt
atgggactttcctacttggcagtacatctactcgaggccacgttctgcttcactctccccatctcccccccctccccacccccaattttgtatttatttattttttaattattt
tgtgcagcgatgggggcggggggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaagcgcgcggggg
cgggagcgggatcagccaccgcggtggcggccctagagtcgatcgaggaactgaaaaaccagaaagttaactggtaagtttagtctttttgtcttttatttcaggt
cccGGCAGCCTTGGAGTGGGTTCCTGCCCCCTCGGGCACACAAACAGAGCTGAAGACCACCCTGG
GCACCTCCTTGGCTGGCCGCATACCTCCTGGCGGGCAGCTGTGtctttcttcttcttcttgcccTGTTCTGGTGG
TACCCAGGGCAAGATCAGGAAGAAAGACACAGAGGCCTGCCTGGCCCTCGAGAGACTGCCCTG
ACTGAAGGCCCTATCAGGTGGGGGAGGGGATCCTGATAGAGGGCACTGCTGCCACTGTTGGGGC
CCAAGgggatccggtggtggtgcaaatcaaagaactgctcctcagtggatgttgcctttacttctaggcctgtacggaagtgttacttctgctctaaaagctgc
ggaattgtacccgcggccgatccaccggtcgccaccatggtgagcaagggcgaggagctgttcaccggggtggtgcccatcctggtcgagctggacggcga
cgtaaacggccacaagttcagcgtgtccggcgagggcgagggcgatgccacctacggcaagctgaccctgaagttcatctgcaccaccggcaagctgcccg
tgccctggcccaccctcgtgaccaccctgacctacggcgtgcagtgcttcagccgctaccccgaccacatgaagcagcacgacttcttcaagtccgccatgcc
cgaaggctacgtccaggagcgcaccatcttcttcaaggacgacggcaactacaagacccgcgccgaggtgaagttcgagggcgacaccctggtgaaccgca
tcgagctgaagggcatcgacttcaaggaggacggcaacatcctggggcacaagctggagtacaactacaacagccacaacgtctatatcatggccgacaagc
agaagaacggcatcaaggtgaacttcaagatccgccacaacatcgaggacggcagcgtgcagctcgccgaccactaccagcagaacacccccatcggcga
cggccccgtgctgctgcccgacaaccactacctgagcacccagtccgccctgagcaaagaccccaacgagaagcgcgatcacatggtcctgctggagttcgt
gaccgccgccgggatcactctcggcatggacgagctgtacaagtaaagcggccatcaagcttatcgataccgtcgactagagctcgctgatcagcctcgactg
tgccttctagttgccagccatctgttgtttgcccctcccccgtgccttccttgaccctggaaggtgccactcccactgtcctttcctaataaaatgaggaaattgcatc
gcattgtctgagtaggtgtcattctattctggggggtggggggggcaggacagcaagggggaggattgggaagacaattaggtagataagtagcatggcgg
gttaatcattaactacaaggaacccctagtgatggagttggccactccctctctgcgcgctcgctcgctcactgaggccgggcgaccaaaggtcgcccgacgcc
cgggctttgcccgggcggcctcagtgagcgagcgagcgcgcag
>human INHBA amiRNA 2-EGFP rAAV (SEQ ID NO: 10)
ctgcgcgctcgctcgctcactgaggccgcccgggcaaagcccgggcgtcgggcgacctttggtcgcccggcctcagtgagcgagcgagcgcgcagagag
ggagtgtagccatgctctaggaagatcaattcggtacaattcacgcgtcgacattgattattgactctggtcgttacataacttacggtaaatggcccgcctggctg
accgcccaacgaccccgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatgcccagtacatgacctt
atgggactttcctacitggcagtacatctactcgaggccacgttctgcttcactctccccatctcccccccctccccacccccaattttgtatttatttattttttaattattt
tgtgcagcgatgggggcggggggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaagcgcgcggggg
cgggagcgggatcagccaccgcggtggcggccctagagtcgatcgaggaactgaaaaaccagaaagttaactggtnagtttagtctttttgtcttttatttcaggt
cccGGCAGCCTTGGAGTGGGTTCCTGCCCCCTCGGGCACACAAACAGAGCTGAAGACCACCCTGG
GCACCTCCTTGGCTGGCCGCATACCTCCTGGCGGGCAGCTGTGtttgccactgtcttctctggaTGTTCTGGTG
GTACCCATCCAGAGATCATAGTGGCAAACACAGAGGCCTGCCTGGCCCTCGAGAGACTGCCCTG
ACTGAAGGCCCTATCAGGTGGGGGAGGGGATCCTGATAGAGGGCACTGCTGCCACTGTTGGGGC
CCAAGgggatccggtggtggtgcaaatcaaagaactgctcctcagtggatgttgcctttacttctaggcctgtacggaagtgttacttctgctctaaaagctgc
ggaattgtacccgcggccgatccaccggtcgccaccatggtgagcaagggcgaggagctgttcaccggggtggtgcccatcctggtcgagctggacggcga
cgtaaacggccacaagttcagcgtgtccggcgagggcgagggcgatgccacctacggcaagctgaccctgaagttcatctgcaccarcggcaagctgcccg
tgccctggcccaccctcgtgaccaccctgacctacggcgtgcagtgcttcagccgctaccccgaccacatgaagcagcacgacttcttcaagtccgccatgcc
cgaaggctacgtccaggagcgcaccatcttcttcaaggacgacggcaactacaagacccgcgccgaggtgaagttcgagggcgacaccctggtgaaccgca
tcgagctgaagggcatcgacttcaaggaggacggcaacatcctggggcacaagctggagtacaactacaacagccacaacgtctatatcatggccgacaagc
agaagaacggcatcaaggtgaacttcaagatccgccacaacatcgaggacggcagcgtgcagctcgccgaccactaccagcagaacacccccatcggcga
cggccccgtgctgctgcccgacaaccactacctgagcacccagtccgccctgagcaaagaccccaacgagaagcgcgatcacatggtcctgctggagttcgt
gaccgccgccgggatcactctcggcatggacgagctgtacaagtaaagcggccatcaagcttatcgataccgtcgactagagctcgctgatcagcctcgactg
tgccttctagttgccagccatctgttgtttgcccctcccccgtgccttccttgaccctggaaggtgccactcccactgtcctttcctaataaaatgaggaaattgcatc
gcattgtctgagtaggtgtcattctattctggggggtggggtggggcaggacagcaagggggaggattgggaagacaattaggtagataagtagcatggcgg
gttaatcattaactacaaggaacccctagtgatggagttggccactccctctctgcgcgctcgctcgctcactgaggccgggcgaccaaaggtcgcccgacgcc
cgggctttgcccgggcggcctcagtgagcgagcgagcgcgcag
>human INHBA amiRNA 3-EGFP rAAV (SEQ ID NO: 11)
ctgcgcgctcgctcgctcactgaggccgcccgggcaazgcccgggcgtcgggcgacctttggtcgcccggcctcagtgagcgagcgagcgcgcagagag
ggagtgtagccatgctctaggaagatcaattcggtacaattcacgcgtcgacattgattattgactctggtcgttacataacttacggtaaatggcccgcctggctg
accgcccaacgaccccgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatgcccagtacatgacctt
atgggactttcctacttggcagtacatctactcgaggccacgttctgcttcactctccccatctcccccccctccccacccccaattttgtatttatttattttttaattattt
tgtgcagcgatgggggcggggggggggggggggggggcgcgcgccaggcggggcggggcggggcgaggggcggggcggggcgaggcggagag
gtgcggcggcagccaatcagagcggcgcgctccgaatgtttccttttatggcgaggcggcggcggcggcggccctataaaaagcgaagcgcgcggggg
cgggagcgggatcagccaccgoggtggcggccctagagtcgatcgaggaactgaaaaaccagasagttaactggtaagtttagtctttttgtcttttatttcaggt
cccGGCAGCCTTGGAGTGGGTTCCTGCCCCCTCGGGCACACAAACAGAGCTGAAGACCACCCTGG
GCACCTCCTTGGCTGGCCGCATACCTCCTGGCGGGCAGCTGTGttcctttcaagtatctctcctTGTTCTGGTGG
TACCCAAGGAGAGAATCCTGAAAGGAACACAGAGGCCTGCCTGGCCCTCGAGAGACTGCCCTGA
CTGAAGGCCCTATCAGGTGGGGGAGGGGATCCTGATAGAGGGCACTGCTGCCACTGTTGGGGCC
CAAGgggatccggtggtggtgcaaatcaaagaactgctcctcagtggatgttgcctttacttctaggcctgtacggaagtgttacttctgctctaaaagctgcg
gaattgtacccgcggccgatccaccggtcgccaccatggtgagcaagggcgaggagctgttcaccggggtggtgcccatcctggtcgagctggacggcgac
gtaaacggccacaagttcagcgtgtccggcgagggcgagggcgatgccacctacggcaagctgaccctgaagttcatctgcaccaccggcaagctgcccgt
gccctggcccaccctcgtgaccaccctgacctacggcgtgcagtgcttcagccgctaccccgaccacatgaagcagcacgacttcttcaagtccgccatgccc
gaaggctacgtccaggagcgcaccatcttcttcaaggacgacggcaactacaagacccgcgccgaggtgaagttcgagggcgacaccctggtgaaccgcat
cgagctgaagggcatcgacttcaaggaggacggcaacatcctggggcacaagctggagtacaactacaacagccacaacgtctatatcatggccgacaagc
agaagaacggcatcaaggtgaacttcaagatccgccacaacatcgaggacggcagcgtgcagctcgccgaccactaccagcagaacacccccatcggcga
cggccccgtgctgctgcccgacaaccactacctgagcacccagtccgccctgagcaaagaccccaacgagaagcgcgatcacatggtcctgctggagttcgt
gaccgccgccgggatcactctcggcatggacgagctgtacaagtaaagcggccatcaagcttatcgataccgtcgactagagctcgctgatcagcctcgactg
tgccttctagttgccagccatctgttgtttgcccctcccccgtgccttccttgaccctggaaggtgccactcccactgtcctttcctaataaaatgaggaaattgcatc
gcattgtctgagtaggtgtcattctattctggggggtggggggggcaggacagcaagggggaggattgggaagacaattaggtagataagtagcatggcgg
gttaatcattaactacaaggaacccctagtgatggagttggccactccctctctgcgcgctcgctcgctcactgaggccgggcgaccaaaggtcgcccgacgcc
cgggctttgcccgggcggcctcagtgagcgagcgagcgcgcag
>human INHBA amiRNA 4-EGFP rAAV (SEQ ID NO: 12)
ctgcgcgctcgctcgctcactgaggccgcccgggcaacgcccgggcgtcgggcgacctttggtcgcccggcctcagtgagcgagcgagcgcgcagagag
ggagtgtagccatgctctaggaagatcaattcggtacaattcacgcgtcgacattgattattgactctggtcgttacataacttacggtaaatggcccgcctggctg
accgcccaacgaccccgcccattgacgtcaataatgacgtatgttcccatagtaacgccaatagggactuccattgacgtcaatgggtggagtantacggtaaa
ctgcccacttggcagtacatcaagtgtatcatatgccaagtacgccccctattgacgtcaatgacggtaaatggcccgcctggcattatgcccagtacatgacctt
atgggactttcctacttggcagtacatctactcgaggccacgttctgcttcactctccccatctcccccccctccccacccccaattttgtatttatttattttttaattattt
tgtgcagcgatgggggcggggggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaagcgcgcggggg
cgggagcgggatcagccaccgcggtggcggccctagagtcgatcgaggaactgaaaaaccagaaagttaactggtaagtttagtctttttgtcttttatttcaggt
cccGGCAGCCTTGGAGTGGGTTCCTGCCCCCTCGGGCACACAAACAGAGCTGAAGACCACCCTGG
GCACCTCCTTGGCTGGCCGCATACCTCCTGGCGGGCAGCTGTGtgatctccgaggtctgctccaTGTTCTGGT
GGTACCCATGGAGCAGTGCCCGGAGATCACACAGAGGCCTGCCTGGCCCTCGAGAGACTGCCCT
GACTGAAGGCCCTATCAGGTGGGGGAGGGGATCCTGATAGAGGGCACTGCTGCCACTGTTGGGG
CCCAAGgggatccggtggtggtgcaaatcaaagaactgctcctcagtggatgttgcctttacttctaggcctgtacggaagtgttacttctgctctanaagctg
cggaattgtacccgcggccgatccaccggtcgccaccatggtgagcaagggcgaggagctgttcaccggggtggtgcccatcctggtcgagctggacggcg
acgtaaacggccacaagttcagcgtgtccggcgagggcgagggcgatgccacctacggcaagctgaccctgaagttcatctgcaccaccggcaagctgccc
gtgccctggcccaccctcgtgaccaccctgacctacggcgtgcagtgcttcagccgctaccccgaccacatgaagcagcacgacttcttcaagtccgccatgc
ccgaaggctagtccaggagcgcaccatcttcttcaaggacgacggcaactacaagacccgcgccgaggtgaagttcgagggcgacaccctggtgaaccgc
atcgagctgaagggcatcgacttcaaggaggacggcaacatcctggggcacaagctggagtacaactacaacagccacaacgtctatatcatggccgacaag
cagaagaacggcatcaaggtgaacttcaagatccgccacaacatcgaggacggcagcgtgcagctcgccgaccactaccagcagaacacccccatcggcg
acggccccgtgctgctgcccgacaaccactacctgagcacccagtccgccctgagcaaagaccccaacgagaagcgcgatcacatggtcctgctggagttc
gtgaccgccgccgggatcactctcggcatggacgagctgtacaagtaaagcggccatcaagcttatcgataccgtcgactagagctcgctgatcagcctcgac
tgtgccttctagttgccagccatctgttgtttgcccctccccgtgccttccttgaccctggaaggtgccactcccactgtcctttcctaataaaatgaggaaattgcat
cgcattgtctgagtaggtgtcattctattctggggggtggggggggcaggacagcaagggggaggattgggaagacaattaggtagataagtagcatggcg
ggttaatcattaactacaaggaacccctagtgatggagttggccactccctctctgcgcgctcgctcgctcactgaggccgggcgaccaaaggtcgcccgacg
cccgggctttgcccgggcggcctcagtgagcgagcgagcgcgcag
>human INHBA amiRNA 5-EGFP rAAV (SEQ ID NO: 13)
ctgcgcgctcgctcgctcactgaggccgcccgggcaaagcccgggcgtcgggcgacctttggtcgcccggcctcagtgagcgagcgagcgcgcagagag
ggagtgtagccatgctctaggaagatcaattcggtacaattcacgcgtcgacattgattattgactctggtcgttacataacttacggtaaatggcccgcctggctg
accgcccaacgaccccgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatgcccagtacatgacctt
atgggactttcctacttggcagtacatctactcgaggccacgttctgcttcactctccccatctcccccccctccccacccccaattttgtatttatttattttttaattattt
gtgcggcggcagccaatcagagcggcgcgctccgaaagtttccttttatggcgaggcggcggcggcggcggccctataaaaagcgaagcgcgcggcggg
cgggagcgggatcagccaccgcggtggggccctagagtcgatcgaggaactgaaaaaccagaaagttaactggtaagtttagtctttttgtcttttatttcaggt
cccGGCAGCCTTGGAGTGGGTTCCTGCCCCCTCGGGCACACAAACAGAGCTGAAGACCACCCTGG
GCACCTCCTTGGCTGGCCGCATACCTCCTGGCGGGCAGCTGTGttttgatgatgttttgaccatTGTTCTGGTGG
TACCCAATGGTCAATTCGTCATCAAAACACAGAGGCCTGCCTGGCCCTCGAGAGACTGCCCTGA
CTGAAGGCCCTATCAGGTGGGGGAGGGGATCCTGATAGAGGGCACTGCTGCCACTGTTGGGGCC
CAAGgggatccggtggtggtgcaaatcaaagaactgctcctcagtggatgttgcctttacttctaggcctgtacggaagtgttacttctgctctaaaagctgcg
gaattgtacccgcggccgatccaccggtcgccaccatggtgagcaagggcgaggagctgttcaccggggtggtgcccatcctggtcgagctggacggcgac
gtaaacggccacaagttcagcgtgtccggcgagggcgagggcgatgccacctacggcaagctgaccctgaagttcatctgcaccaccggcaagctgcccgt
gccctggcccaccctcgtgaccaccctgacctacggcgtgcagtgcttcagccgctaccccgaccacatgaagcagcacgacttcttcaagtccgccatgccc
gaaggctacgtccaggagcgcaccatcttcttcaaggacgacggcaactacaagacccgcgccgaggtgaagttcgagggcgacaccctggtgaaccgcat
cgagctgaagggcatcgacttcaaggaggacggcaacatcctggggcacaagctggagtacaactacaacagccacaacgtctatatcatggccgacaagc
agaagaacggcatcaaggtgaacttcaagatccgccacaacatcgaggacggcagcgtgcagctcgccgaccactaccagcagaacacccccatcggcga
cggccccgtgctgctgcccgacaaccactacctgagcacccagtccgccctgagcaaagaccccaacgagaagcgcgatcacatggtcctgctggagttcgt
gaccgccgccgggatcactctcggcatggacgagctgtacaagtaaagcggccatcaagcttatcgataccgtcgactagagctcgctgatcagcctcgactg
tgccttctagttgccagccatctgttgtttgcccctcccccgtgccttccttgaccctggaaggtgccactcccactgtcctttcctaataaaatgaggaaattgcatc
gttaatcattaactacaaggaacccctagtgatggagttggccactccctctctgcgcgctcgctcgctcactgaggccgggcgaccaaaggtcgcccgacgcc
gcattgtctgagtaggtgtcattctattctggggggtggggggggcaggacagcaagggggaggattgggaagacaattaggtagataagtagcatggcgg
cgggctttgcccgggcggcctcagtgagcgagcgagcgcgcag
>human INHBA amiRNA 6-EGFP rAAV (SEQ ID NO: 14)
ctgcgcgctcgctcgctcactgaggccgcccgggcaaagcccgggcgtcgggcgacctttggtcgcccggcctcagtgagcgagcgagcgcgcagagag
ggagtgtagccatgctctaggaagatcaattcggtacaattcacgcgtcgacattgattattgactctggtcgttacataacttacggtaaatggcccgcctggctg
accgcccaacgaccccgcccattgacgtcaataatgacgtatgttcccatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattatgcccagtacatgacctt
atgggactttcctacttggcagtacatctactcgaggccacgttctgcttcactctccccatctcccccccctccccacccccaattttgtatttatttattttttaattattt
tgtgcagcgatgggggcggggggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaagcgcgcggcggg
cgggagcgggatcagccaccgcggtggcggccctagagtcgatcgaggaactgaaaaaccagaaagttaactggtaagtttagtctttttgtctittatttcaggt
cccGGCAGCCTTGGAGTGGGTTCCTGCCCCCTCGGGCACACAAACAGAGCTGAAGACCACCCTGG
GCACCTCCTTGGCTGGCCGCATACCTCCTGGGGGGCAGCTGTGtctttcttcttcttcttgcccTGTTCTGGTGG
TACCCAGGGCAAGATCAGGAAGAAAGACACAGAGGCCTGCCTGGCCCTCGAGAGACTGCCCTG
ACTGAAGGCCCTATCAGGTGGGGGAGGGGATCCTGATAGAGGGCACTGCTGCCACTGTTGGGGC
CCAAGgggatccggtggtggtgcaaatcaaagaactgctcctcagtggatgttgcctttacttctaggcctgtacggaagtgttacttctgctctaaaagctgc
ggaattgtacccgcggccgatccaccggtcgccaccatggtgagcaagggcgaggagctgttcaccggggggtgcccatcctggtcgagctggacggcga
cgtaaacggccacaagttcagcgtgtccggcgagggcgagggcgatgccacctacggcaagctgaccctgaagttcatctgcaccaccggcaagctgcccg
tgccctggcccaccctcgtgaccaccctgacctacggcgtgcagtgcttcagccgctaccccgaccacatgaagcagcacgacttcttcaagtccgccatgcc
cgaaggctacgtccaggagcgcaccatcttcttcaaggacgacggcaactacaagacccgcgccgaggtgaagttcgagggcgacaccctggtgaaccgca
tcgagctgaagggcatcgacttcaaggaggacggcaacatcctggggcacaagctggagtacaactacaacagccacaacgtctatatcatggccgacaagc
agaagaacggcatcaaggtgaacttcaagatccgccacaacatcgaggacggcagcgtgcagctcgccgaccactaccagcagaacacccccatcggcga
cggccccgtgctgctgcccgacaaccactacctgagcacccagtccgccctgagcaaagaccccaacgagaagcgcgatcacatggtcctgctggagttcgt
gaccgccgccgggatcactctcggcatggacgagctgtacaagtaaagcggccatcaagcttatcgataccgtcgactagagctcgctgatcagcctcgactg
tgccttctagttgccagccatctgtigtttgcccctcccccgtgccttccttgaccctggaaggtgccactcccactgtcctttcctaataaaatgaggaaattgcatc
gcattgtctgagtaggtgtcattctattctggggggtggggggggcaggacagcaagggggaggattgggaagacaattaggtagataagtagcatggcgg
gttaatcattaactacaaggaacccctagtgatggagttggccactccctctctgcgcgctcgctcgctcactgaggccgggcgaccaaaggtcgcccgacgcc
cgggctttgcccgggcggcctcagtgagcgagcgagcgcgcag
>pAAV. EndoID1-BRE NFKB. amiR-33-ACVR1-INHBA-EGFP.miR-122TS (SEQ ID NO: 15)
ctgcgcgctcgctcgctcactgaggccgcccgggcaaagcccgggcgtcgggcgacctttggtcgcccggcctcagtgagcgagcgagcgcgcagagag
ggagtggccaactccatcactaggggttccttgtagttaatgattaacccgccatgctacttatctaccagggtaatggggatcctCTAGCactatagctagtc
gacattgattattgactagttattaatagtaatcaattacggggtcattagttcatagcccatatatggagttccgcgttacataacttacggtaaatggcccgcctggc
tgaccgcccaacgacccccgcccattgacgtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatgtcgaggGCTTCGCGCCCTAAGTCTGCAGGTGAC
GGGCTCAGGGGCGGGGGCTGGGTGGGGGGGAGCGGAGAATGCTCCAGCCCAGTTTGCCGTCTCC
ATGGCGACCGCCCGCGCGGCGCCAGCCTGACAGCCCGTCCGGGTTTTATGAATGGGTGACGTCA
CGGGCCTGGCGTCTAACGGTCTGAGCCGCTTGTTCAGACGCTGACACAGACCAGCCCGGGAAAG
GTGAGCTCACAGAGGGGACTTTCCGAGAGATCTACAGAGGGGACTTTCCGAGAGCGAGCTTGGG
CTGCAGGTCGACCGTCCATCCATTCACAGCGCTTCTATAAAGGCGCCAGCTGAGGCGCCTACTAC
TCCAACCGCGACTGCAGCGAGCAACTGAGAAGACTGGATAGAGCCGGCGGTTCCGCGAACGAG
CAGTGACCGCGCTCCCACCCAGCTCTGCTCTGCAGCTCCACCAGTGTCTCTCTAGAcgggagcaagctttc
agatcgcctggagacgccatccacgctgttttgacctccatagaagacaccgggaccgatccagcctccgcggccgggaacggtgcattggaacgcggattc
cccgtgccaagagtgacgtaagtaccgcctatagagtctataggcccacccccttggcttcttatgcatgctatactgtttttggcttggggtctatacacccccgct
tcctcatgttTGCTGCCCGTGACCAGCACGTCAACGATTTTGTGGGCACGGGCGACACcgcagtgtagtctgagca
gtactcgttgctgccgcgcgcgccaccagacataatagctgacagactaacagactgttcctttccatgggtcttttctgcagtcaccgtcgccgccAGGGC
TCTGCGTTTGCTCCAGGTAGTCCGCTGCTCCCTTGGGCCTGGGCCCACTGACAGCCCTGGTGCCT
CTGGCCGGCTGCACACCTCCTGGCGGGCAGCTGTGtgtaatctggtgagccactgtTGTTCTGGCAATACCTG
ACAGTGGCAGATCAGATTACACACGGAGGCCTGCCCTGACTGCCCACGGTGCCGTGGCCAAAGA
GGATCTAAGGGCACCGCTGAGGGCCTACCTAACCATCGTGGGGAATAAGGACAGTGTCACCCAT
TTAAATGGCAGCCTTGGAGTGGGTTCCTGCCCCCTCGGGCACACAAACAGAGCTGAAGACCACC
CTGGGCACCTCCTTGGCTGGCCGCATACCTCCTGGCGGGCAGCTGTGtgatctccgaggtctgctccaTGTTCT
GGTGGTACCCATGGAGCAGTGCCCGGAGATCACACAGAGGCCTGCCTGGCCCTCGAGAGACTGC
GGGCCCAAGACCGGtgaattccgccaccatggtgagcaagggcgaggagctgttcaccggggtggtgcccatcctggtcgagctggacggcga
CCTGACTGAAGGCCCTATCAGGTGGGGGAGGGGATCCTGATAGAGGGCACTGCTGCCACTGTTG
cgtaaacggccacaagttcagcgtgtccggcgagggcgagggcgatgccacctacggcaagctgaccctgaagttcatctgcaccaccggcaagctgcccg
tgccctggcccaccctcgtgaccaccctgacctacggcgtgcagtgcttcagccgctaccccgaccacatgaagcagcacgacttcttcaagtccgccatgcc
cgaaggctacgtccaggagcgcaccatcttcttcaaggacgacggcaactacaagacccgcgccgaggtgaagttcgagygcgacaccctggtgaaccgca
tcgagctgaagggcatcgacttcaaggaggacggcaacatcctggggcacaagctggagtacaactacaacagccacaacgtctatatcatggccgacaagc
agaagaacggcatcaaggtgaacttcaagatccgccacaacatcgaggacggcagcgtgcagctcgccgaccactaccagcagaacacccccatcggcga
cggccccgtgctgctgcccgacaaccactacctgagcacccagtccgccctgagcaaagaccccaacgagaagcgcgatcacatggtcctgctggagttcgt
gaccgccgccgggatcactctcggcatggacgagctgtacaagtaaccTgAacaaacaccattgtcacactccaacaaacaccattgtcacactccaacaaa
caccattgtcacactccatgaggatccgatctttttccctctgccaaaaattatggggacatcatgaagccccttgagcatctgacttctggctaataaaggaaattta
ttttcattgcaatagtgtgttggaattttttgtgtctctcactcggaagcaattcgttgatctgaatttcgaccacccataatacccattaccctggtagataagtagcatg
gcgggttaatcattaactacaaggaacccctagtgatggagttggccactccctctctgcgcgctcgctcgctcactgaggccgggcgaccaaaggtcgcccg
acgcccgggcittgcccgggcggcctcagtgagcgagcgagcgcgcag
>pAA Vss-CB-PI(MBL-amiRACVR1-INHBA)-opt-ACVR1-miR-122T (SEQ ID NO: 16)
ctgcgcgctcgctcgctcactgaggccgcccgggcaaagcccgggcgtcgggcgacctttggtcgcccggcctcagtgagcgagcgagcgcgcagagag
ggagtggccaactccatcactaggggttccttgtagttaatgattaacccgccatgctacttatctaccagggtaatggggatcctctagaactatagctagtcgac
attgattattgactagttattaatagtaatcaattacggggtcattagttcatagcccatatatggagttccgcgttacataacttacggtaaatggcccgcctggctga
ccgcccaacgacccccgcccattgacgtcaataatgacgtatgttcccatagtaacgccaatagggactttccattgacgtcaatgggggagtatttacggtaaa
ctgcccacttggcagtacatcaagtgtatcatatgccaagtacgccccctattgacgtcaatgacggtaaatggcccgcctggcattatgcccagtacatgacctt
atgggactttcctacttggcagtacatctacgtattagtcatcgctattaccatgtcgaggccacgttctgcttcactctccccatctcccccccctccccacccccaa
gaggcggagaggtgcggcggcagccaatcagagcggcgcgctccgaaagtttccttttatggcgaggcggcggcggcggcggccctataaaaagcgaag
cgcgcggcgggcgggagcaagctttcagatcgcctggagacgccatccacgctgttttgacctccatagaagacaccgggaccgatccagcctccgcggcc
gggaacggtgcattggaacgcggattccccgtgccaagagtgacgtaagtaccgcctatagagtctataggcccacccccttggcttcttatgcatgctatactgt
ttttggcttggggtctatacacccccgcttcctcatgttTGCTGCCCGTGACCAGCACGTCAACGATTTTGTGGGCACGGG
CGACACcgcagtgtagtctgagcagtactcgttgctgccgcgcgcgccaccagacataatagctgacagactaacagactgttcctttccatgggtcttttct
gcaAGGGCTCTGCGTTTGCTCCAGGTAGTCCGCTGCTCCCTTGGGCCTGGGCCCACTGACAGCCCT
ATACCTGACAGTGGCAGATCAGATTACACACGGAGGCCTGCCCTGACTGCCCACGGTGCCGTGG
GGTGCCTCTGGCCGGCTGCACACCTCCTGGCGGGCAGCTGTGtgtaatctggtgagccactgtTGTTCTGGCA
CCAAAGAGGATCTAAGGGCACCGCTGAGGGCCTACCTAACCATCGTGGGGAATAAGGACAGTGT
CACCCtgcagtcaccgtcgccgccaccggGGCAGCCTTGGAGTGGGTTCCTGCCCCCTCGGGCACACAAACA
GAGCTGAAGACCACCCTGGGCACCTCCTTGGCTGGCCGCATACCTCCTGGCGGGCAGCTGTGtgatc
tccgaggtctgctccaTGTTCTGGTGGTACCCATGGAGCAGTGCCCGGAGATCACACAGAGGCCTGCCTGG
CTGCTGCCACTGTTGGGGCCCAAGtgaattccgccaccatggtcgatggagtgatgatcctgcctgtcctgattatgattgccctgcccagc
CCCTCGAGAGACTGCCCTGACTGAAGGCCCTATCAGGTGGGGGAGGGGATCCTGATAGAGGGCA
cccagcatggaagatgaaaaacctaaagtcaaccctaagctgtatatgtgcgtgtgcgagggcctgagctgcggaaacgaggatcactgcgagggccagca
gtgtttcagctccctgtccatcaatgacggcttccacgtgtaccagaagggctgctttcaggtgtatgagcagggcaagatgacctgtaagacaccaccttcccca
ggacaggcagiggagtgctgtcagggcgattggtgtaaccggaatatcaccgcccagctgccaacaaagggcaagtctttccccggcacacagaactttcacc
tggaagtgggcctgatcatcctgagcgtggtgttcgccgtgtgcctgctggcatgtctgctgggagtggccctgagaaagtttaagcggagaaaccaggagcg
gctgaatccaagagatgtggagtacggcaccatcgagggcctgatcaccacaaatgtgggcgactctacactggccgacctgctggatcacagctgcaccag
cggctccggatctggcctgccctttctggtgcagaggaccgtggcccggcagatcaccctgctggagtgcgtgggcaagggccggtacggagaagtgtgga
gaggatcctggcagggagagaacgtggcagtgaagatcttctctagccgggatgagaagtcttggtttagagagacagagctgtataacacagtgatgctgag
gcacgagaatatcctgggcttcatcgcctccgacatgacctctcgccactcctctacacagctgtggctgatcacccactaccacgagatgggctccctgtacga
ttacctccagctgaccacactggacacagtgtcttgcctgcggatcgtgctgtctatcgccagcggcctggcacacctgcacatcgagatctttggaacccaggg
caagccagcaatcgcacacagagatctgaagtctaagaacatcctggtgaagaagaatggccagtgctgtatcgccgatctgggcctggccgtgatgcacagc
cagtccaccaaccagctggacgtgggcaacaatcctcgggtgggcacaaagagatacatggccccagaggtgctggatgagacaatccaggtggactgcttc
gatagctataagagggtggacatctgggcctttggcctggtgctgtgggaggtggcaaggaggatggtgagcaacggcatcgtggaggactacaagccaccc
ttctatgacgtggtgcctaatgatccatcctttgaggacatgcgcaaggtggtgtgcgtggatcagcagaggcccaacatccctaatcgctggttcagcgacccc
accctgacatccctggccaagctgatgaaggagtgttggtatcagaatcctagcgccaggctgaccgccctgcgcatcaagaaaactctgactaaaatcgacaa
tagcctggatacactgaaaaccgactgctgaccTgAacaaacaccattgtcacactccaacaaacaccattgtcacactccaacaaacaccattgtcacactcc
atgaggatccgatctttttccctctgccaaaaattatggggacatcatgaagccccttgagcatctgacttctggctaataaaggaaatttattttcattgcaatagtgt
gttggaattttttgtgtctctcactcggaagcaattcgttgatctgaatttcgaccacccataatacccattaccctggtagataagtagcatgggggttaatcattaa
ctacaaggaacccctagtgatggagttggccactccctctctgcgcgctcgctcgctcactgaggccgggcgaccaaaggtcgcccgacgcccgggctttgc
ccgggcggcctcagtgagcgagcgagcgcgcag
>pAAV. EndoID1-BRE NFKB. amiR-33-ACVR1-INHBA-STNFR2.miR-122TS (SEQ ID NO: 17)
ctgcgcgctcgctcgctcactgaggccgcccgggcaaagcccgggcgtcgggcgacctttggtcgcccggcctcagtgagcgagcgagcgcgcagagag
ggagtggccatctccatcactaggggttccttgtagttaatgattaacccgccatgctacttatctaccagggtaatggggatcctCTAGCactatagctagtc
gacattgattattgactagttattaatagtaatcaattacggggtcattagttcatagcccatatatggagitccgcgttacataacttacggtaaatggcccgcctggc
tgaccgcccaacgacccccgcccattgacgtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatgtcgaggGCTTCGCGCCCTAAGTCTGCAGGTGAC
GGGCTCAGGGGCGGGGGCTGGGTGGGGGGGAGCGGAGAATGCTCCAGCCCAGTTTGCCGTCTCC
ATGGCGACCGCCCGCGCGGCGCCAGCCTGACAGCCCGTCCGGGTTTTATGAATGGGTGACGTCA
CGGGCCTGGCGTCTAACGGTCTGAGCCGCTTGTTCAGACGCTGACACAGACCAGCCCGGGAAAG
GTGAGCTCACAGAGGGGACTTTCCGAGAGATCTACAGAGGGGACTTTCCGAGAGCGAGCTTGGG
CTGCAGGTCGACCGTCCATCCATTCACAGCGCTTCTATAAAGGCGCCAGCTGAGGCGCCTACTAC
TCCAACCGCGACTGCAGCGAGCAACTGAGAAGACTGGATAGAGCCGGCGGTTCCGCGAACGAG
CAGTGACCGCGCTCCCACCCAGCTCTGCTCTGCAGCTCCACCAGTGTCTCTCTAGAcgggagcaagctttc
agatcgcctggagacgccatccacgctgttttgacctccatagaagacaccgggaccgatccagcctccgcggccgggaacggtgcattggaacgcggattc
cccgtgccaagagtgacgtaagtaccgcctatagagtctataggcccacccccttggcttcttatgcatgctatactgtttttggcttggggtctatacacccccgct
tcctcatgttTCCTGCCCGTGACCAGCACGTCAACGATTTTGTGGGCACGGGCGACACcgcagtgtagtctgagca
gtactcgttgctgccgcgcgcgccaccagacataatagctgacagactaacagactgttcctttccatgggtcttttctgcagtcaccgtcgccgccAGGGC
TCTGCGTTTGCTCCAGGTAGTCCGCTGCTCCCTTGGGCCTGGGCCCACTGACAGCCCTGGTGCCT
CTGGCCGGCTGCACACCTCCTGGCGGGCAGCTGTGtgtaatctggtgagccactgtTGTTCTGGCAATACCTG
ACAGTGGCAGATCAGATTACACACGGAGGCCTGCCCTGACTGCCCACGGTGCCGTGGCCAAAGA
GGATCTAAGGGCACCGCTGAGGGCCTACCTAACCATCGTGGGGAATAAGGACAGTGTCACCCAT
TTAAATGGCAGCCTTGGAGTGGGTTCCTGCCCCCTCGGGCACACAAACAGAGCTGAAGACCACC
CTGGGCACCTCCTTGGCTGGCCGCATACCTCCTGGCGGGCAGCTGTGtgatctccgaggtctgctccaTGTTCT
GGTGGTACCCATGGAGCAGTGCCCGGAGATCACACAGAGGCCTGCCTGGCCCTCGAGAGACTGC
CCTGACTGAAGGCCCTATCAGGTGGGGGAGGGGATCCTGATAGAGGGCACTGCTGCCACTGTTG
GGGCCCAAGACCGGtGCCACCatggcgcccgtcgccgtctgggccgcgctggccgtcggactggagctctgggctgcggcgcacgcctt
gcccgcccaggtggcatttacaccctacgccccggagcccgggagcacatgccggctcagagaatactatgaccagacagctcagatgtgctgcagcaaatg
ctcgccgggccaacatgcaaaagtcttctgtaccaagacctcggacaccgtgtgtgactcctgtgaggacagcacatacacccagctctggaactgggttcccg
agtgcttgagctgtggctcccgctgtagctctgaccaggtggaaactcaagcctgcactcgggaacagaaccgcatctgcacctgcaggcccggctggtactg
cgcgctgagcaagcaggaggggtgccggctgtgcgcgccgctgcgcaagtgccgcccgggcttcggcgtggccagaccaggaactgaaacatcagacgt
ggtgtgcaagccctgtgccccggggacgttctccaacargacttcatccacggatatttgcaggccccaccagatctgtaacgtggtggccatccctgggaatg
caagcatggatgcagtctgcacgtccacgtcccccacccggagtatggccccaggggcagtacacttaccccagccagtgtccacacgatcccaacacacgc
agccaactccagaacccagcactgctccaagcacctccttcctgctcccaatgggccccagccccccagctgaagggagcactggcgacttcgctcttccagtt
tgatgtacaagtaacgTgAacasacaccattgtcacactccaacaaacaccattgtcacactccaacaaacaccattgtcacactccatgaggatccgatcttttt
ccctctgccaaaaattatggggacatcatgaagccccttgagcatctgacttctggctaataaaggaaatttattttcattgcaatagtgtgttggaattttttgtgtctct
cactcggaagcaattcgttgatctgaatttcgaccacccataatacccattaccctggtagataagtagcatggcgggttaatcattaactacaaggaacccctagt
gatggagttggccactccctctctgcgcgctcgctcgctcactgaggccgggcgaccaaaggtcgcccgacgcccgggctttgcccgggcggcctcagtga
gcgagcgagcgcgcag
>pAAV. EndoID1-BRE NFKB. amiR-33-ACVR1-INHBA-sIL1Ra.miR-122TS (SEQ ID NO: 18)
ctgcgcgctcgctcgctcactgaggccgcccgggcaacgcccgggcgtcgggcgacctttggtcgcccggcctcagtgagcgagcgagcgcgcagagag
ggagtggccasctccatcactaggggttccttgtagttaatgattaacccgccatgctacttatctaccagggtaatggggatcctCTAGCactatagctagtc
gacattgattattgactagtrattaatagtaatcaattacggggtcattagttcatagcccatatatggagttccgcgttacataacttacggtaaatggcccgcctggc
tgaccgcccaacgacccccgcccattgacgtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatgtcgaggGCTTCGCGCCCTAAGTCTGCAGGTGAC
GGGCTCAGGGGCGGGGGCTGGGTGGGGGGGAGCGGAGAATGCTCCAGCCCAGTTTGCCGTCTCC
ATGGCGACCGCCCGCGCGGCGCCAGCCTGACAGCCCGTCCGGGTTTTATGAATGGGTGACGTCA
CGGGCCTGGCGTCTAACGGTCTGAGCCGCTTGTTCAGACGCTGACACAGACCAGCCCGGGAAAG
GTGAGCTCACAGAGGGGACTTTCCGAGAGATCTACAGAGGGGACTTTCCGAGAGCGAGCTTGGG
CTGCAGGTCGACCGTCCATCCATTCACAGCGCTTCTATAAAGGCGCCAGCTGAGGCGCCTACTAC
TCCAACCGCGACTGCAGCGAGCAACTGAGAAGACTGGATAGAGCCGGCGGTTCCGCGAACGAG
CAGTGACCGCGCTCCCACCCAGCTCTGCTCTGCAGCTCCACCAGTGTCTCTCTAGAcgggagcaagctttg
agatcgcctggagacgccatccacgctgttttgacctccatagaagacaccgggaccgatccagcctccgcggccgggaacggtgcattggaacgcggattg
cccgtgccaagagtgacgtaagtaccgcctatagagtctataggcccacccccttggcttcttatgcatgctatactgtttttggcttggggtctatacacccccgct
tcctcatgttTCCTGCCCGTGACCAGCACGTCAACGATTTTGTGGGCACGGGCGACACcgcagtgtagtctgagca
gtactcgttgctgccgcgcgcgccaccagacataatagctgacagactaacagactgttcctttccatgggtcttttctgcagtcaccgtcgccgccAGGGC
TCTGCGTTTGCTCCAGGTAGTCCGCTGCTCCCTTGGGCCTGGGCCCACTGACAGCCCTGGTGCCT
CTGGCCGGCTGCACACCTCCTGGCGGGCAGCTGTGtgtaatctggtgagccactgtTGTTCTGGCAATACCTG
ACAGTGGCAGATCAGATTACACACGGAGGCCTGCCCTGACTGCCCACGGTGCCGTGGCCAAAGA
GGATCTAAGGGCACCGCTGAGGGCCTACCTAACCATCGTGGGGAATAAGGACAGTGTCACCCAT
TTAAATGGCAGCCTTGGAGTGGGTTCCTGCCCCCTCGGGCACACAAACAGAGCTGAAGACCACC
GGGCCCAAGACCGGtGCCACCatggaaatctgcagaggcctccgcagtcacctaatcactctcctcctcttcctgttccattcagagacgatct
CTGGGCACCTCCTTGGCTGGCCGCATACCTCCTGGCGGGCAGCTGTGtgatctccgaggtctgctccaTGTTCT
GGTGGTACCCATGGAGCAGTGCCCGGAGATCACACAGAGGCCTGCCTGGCCCTCGAGAGACTGC
CCTGACTGAAGGCCCTATCAGGTGGGGGAGGGGATCCTGATAGAGGGCACTGCTGCCACTGTTG
gccgaccctctgggagaaaatccagcaagatgcaagccttcagaatctgggatgttaaccagaagaccttctatctgaggaacaaccaactagttgctggatact
tgcaaggaccaaatgtcaatttagaagaaaagatagatgtggtacccattgagcctcatgctctgttcttgggaatccatggagggaagatgtgcctgtcctgtgtc
aagtctggtgatgagaccagactccagctggaggcagttaacatcactgacctgagcgagaacagaaagcaggacaagcgcttcgccttcatccgctcagac
agtggccccaccaccagttttgagtctgccgcctgccccggttggttcctctgcacagcgatggaagctgaccagcccgtcagcctcaccaatatgcctgacga
aggcgtcatggtcaccaaattctacttccaggaggacgagtagtaatgtacaagtaacgTgAacacacaccattgtcacactccaacaaacaccattgtcacac
tccaacaaacaccattgtcacactccatgaggatccgatctttttccctctgccaaaaattatggggacatcatgaagccccttgagcatctgacttctggctaataa
aggaaatttatttcattgcaatagtgtgttggaattttttgtgtctctcactcggaagcaattcgttgatctgaatttcgaccacccataatacccattaccctggtagat
aagtagcatggcgggttaatcattaactacaaggaacccctagtgatggagttggccactccctctctgcgcgctcgctcgctcactgaggccgggcgaccaaa
ggtcgcccgacgcccgggctttgcccgggcggcctcagtgagcgagcgagcgcgcag
>pAAV. EndoID1-BRE NFKB. amiR-33-ACVR1-INHBA-sILIRa. sTNFR2.miR-122TS (SEQ ID NO: 19)
ctgcgcgctcgctcgctcactgaggccgcccgggcaazgcccgggcgtcgggcgacctttggtcgcccggcctcagtgagcgagcgagcgcgcagagag
ggagtggccaactccatcactaggggttccttgtagttaatgattaacccgccatgctacttatctaccagggtaatggggatcctCTAGCactatagctagtc
gacattgattattgactagttattaatagtaatcaattacggggtcattagttcatagcccatatatggagttccgcgttacataacttacggtaaatggcccgcctggc
tgaccgcccaacgacccccgcccattgacgtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatgtcgaggGCTTCGCGCCCTAAGTCTGCAGGTGAC
GGGCTCAGGGGCGGGGGCTGGGTGGGGGGGAGCGGAGAATGCTCCAGCCCAGTTTGCCGTCTCC
ATGGCGACCGCCCGCGCGGCGCCAGCCTGACAGCCCGTCCGGGTTTTATGAATGGGTGACGTCA
CGGGCCTGGCGTCTAACGGTCTGAGCCGCTTGTTCAGACGCTGACACAGACCAGCCCGGGAAAG
GTGAGCTCACAGAGGGGACTTTCCGAGAGATCTACAGAGGGGACTTTCCGAGAGCGAGCTTGGG
CTGCAGGTCGACCGTCCATCCATTCACAGCGCTTCTATAAAGGCGCCAGCTGAGGCGCCTACTAC
TCCAACCGCGACTGCAGCGAGCAACTGAGAAGACTGGATAGAGCCGGCGGTTCCGCGAACGAG
CAGTGACCGCGCTCCCACCCAGCTCTGCTCTGCAGCTCCACCAGTGTCTCTCTAGAcgggagcaagctttc
agatcgcctggagacgccatccacgctgttttgacctccatagaagacaccgggaccgatccagcctccgcggccgggaacggtgcattggaacgcggattc
cccgtgccaagagtgacgtaagtaccgcctatagagtctataggcccacccccttggcttcttatgcatgctatactgtttttggcttggggtctatacacccccgct
tcctcatgttTCCTGCCCGTGACCAGCACGTCAACGATTTTGTGGGCACGGGCGACACcgcagtgtagtctgagca
gtactcgttgctgccgcgcgcgccaccagacataatagctgacagactaacagactgttcctttccatgggtcttttctgcagtcaccgtcgccgccAGGGC
TCTGCGTTTGCTCCAGGTAGTCCGCTGCTCCCTTGGGCCTGGGCCCACTGACAGCCCTGGTGCCT
CTGGCCGGCTGCACACCTCCTGGCGGGCAGCTGTGtgtaatctggtgagccactgtTGTTCTGGCAATACCTG
ACAGTGGCAGATCAGATTACACACGGAGGCCTGCCCTGACTGCCCACGGTGCCGTGGCCAAAGA
GGATCTAAGGGCACCGCTGAGGGCCTACCTAACCATCGTGGGGAATAAGGACAGTGTCACCCAT
TTAAATGGCAGCCTTGGAGTGGGTTCCTGCCCCCTCGGGCACACAAACAGAGCTGAAGACCACC
CTGGGCACCTCCTTGGCTGGCCGCATACCTCCTGGCGGGCAGCTGTGtgatctccgaggtctgctccaTGTTCT
GGTGGTACCCATGGAGCAGTGCCCGGAGATCACACAGAGGCCTGCCTGGCCCTCGAGAGACTGC
CCTGACTGAAGGCCCTATCAGGTGGGGGAGGGGATCCTGATAGAGGGCACTGCTGCCACTGTTG
GGGCCCAAGACCGGtGCCACCatggaaatctgcagaggcctccgcagtcacctaatcactctcctcctcttcctgttccattcagagacgatct
gccgaccctctgggagaaaatccagcaagatgcaagccttcagaatctgggatgttaaccagaagaccttctatctgaggaacaaccaactagttgctggatact
tgcaaggaccaaatgtcaatttagaagaaaagatagatgtggtacccattgagcctcatgctctgttcttgggaatccatggagggaagatgtgcctgtcctgtgtc
aagtctggtgatgagaccagactccagctggaggcagttaacatcactgacctgagcgagaacagaaagcaggacaagcgcttcgccttcatccgctcagac
agtggccccaccaccagttttgagtctgccgcctgccccggttggttcctctgcacagcgatggaagctgaccagcccgtcagcctcaccaatatgcctgacga
aggcgtcatggtcaccaaattctacttccaggaggacgagggatccggagagggcagaggaagtctgctaacatgcggtgacgtcgaggagaatcctggac
ctatggcgcccgtcgccgtctgggccgcgctggccgtcggactggagctctgggctgcggcgcacgccttgcccgcccaggtggcatttacaccctacgccg
cggagcccgggagcacatgccggctcagagaatactatgaccagacagctcagatgtgctgcagcaaatgctcgccgggccaacatgcaaaagtcttctgta
ccaagacctcggacaccgtgtgtgactcctgtgaggacagcacatacacccagctctggaactggcttcccgagtgcttgagctgtggctcccgctgtagctct
gaccaggtggaaactcaagcctgcactcgggaacagaaccgcatctgcacctgcaggcccggctggtactgcgcgctgagcaagcaggaggggtgccggg
tgtgcgcgccgctgcgcaagtgccgcccgggcttcggcgtggccagaccaggaactgaaacatcagacgtggtgtgcaagccctgtgccccggggacgttc
tccaacacgacttcatccacggatatttgcaggccccaccagatctgtaacgtggtggccatccctgggaatgcaagcatggatgcagtctgcacgtccacgtcc
cccacccggagtatggccccaggggcagtacacttaccccagccagtgtccacacgatcccaacacacgcagccaactccagaacccagcactgctccaag
cacctccttcctgctcccaatgggccccagccccccagctgaagggagcactggcgacttcgctcttccagtttgattacaagtaaccTgAacaaacaccatt
gtcacactccaacaaacaccattgtcacactccaacaaacaccattgtcacactccatgaggatccgatctttttccctctgccaaaaattatggggacatcatgaa
gccccttgagcatctgacttctggctaataaaggaaatttattttcattgcaatagtgtgttggaattttttgtgtctctcactcggaagcaattcgttgatctgaatttcga
ccacccataatacccattaccctggtagataagtagcatggcgggttaatcattaactacaaggaacccctagtgatggagttggccactccctctctgcgcgctg
gctcgctcactsaggccgggcgaccaaaggtcgcccgacgcccgggctttgcccgggggcctcagtgagcgagcgagcgcgcag
>pAAV. EndoID1-BRE NFKB. sIL-1Ra-activin A trap(LN).miR-122TS (SEQ ID NO: 20)
ctgcgcgctcgctcgctcactgaggccgcccgggcaacgcccgggcgtcgggcgacctttggtcgcccggcctcagtgagcgagcgagcgcgcagagag
ggagtggccacctccatcactaggggttccttgtagttaatgattaacccgccatgctacttatctaccagggtaatggggatcctCTAGCactatagctagtg
gacattgattattgactagttattaatagtaatcaattacggggtcattagttcatagcccatatatggagttccgcgttacataacttacggtaaatggcccgcctggc
tgaccgcccaacgacccccgcccattgacgtcaataatgacgtatgttcccatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatgtcgaggGCTTCGCGCCCTAAGTCTGCAGGTGAC
GGGCTCAGGGGCGGGGGCTGGGTGGGGGGGAGCGGAGAATGCTCCAGCCCAGTTTGCCGTCTCC
ATGGCGACCGCCCGCGCGGCGCCAGCCTGACAGCCCGTCCGGGTTTTATGAATGGGTGACGTCA
CGGGCCTGGCGTCTAACGGTCTGAGCCGCTTGTTCAGACGCTGACACAGACCAGCCCGGGAAAG
GTGAGCTCACAGAGGGGACTTTCCGAGAGATCTACAGAGGGGACTTTCCGAGAGCGAGCTTGGG
CTGCAGGTCGACCGTCCATCCATTCACAGCGCTTCTATAAAGGCGCCAGCTGAGGCGCCTACTAC
TCCAACCGCGACTGCAGCGAGCAACTGAGAAGACTGGATAGAGCCGGCGGTTCCGCGAACGAG
CAGTGACCGCGCTCCCACCCAGCTCTGCTCTGCAGCTCCACCAGTGTCTCTCTAGAcgggagcaagctttc
agatcgcctggagacgccatccacgctgttttgacctccatagaagacaccgggaccgatccagcctccgcggccgggaacggtgcattggaacgcggattc
cccgtgccaagagtgacgtaagtaccgcctatagagtctataggcccacccccttggcttcttatgcatgctatactgtttttggcttggggtctatacacccccgct
tcctcatgttTCCTGCCCGTGACCAGCACGTCAACGATTTTGTGGGCACGGGCGACACcgcagtgtagtctgagca
gtactcgttgctgccgcgcgcgccaccagacataatagctgacagactaacagactgttcctttccatgggtcttttctgcagtcaccgtcgccgccaccggtgaa
ttccgccaccatggaaatctgcagaggcctccgcagtcacctaatcactctcctcctcttcctgttccattcagagacgatctgccgaccctctgggagaaaatcc
agcaagatgcaagccttcagaatctgggatgttaaccagaagaccttctatctgaggaacaaccaactagttgctggatacttgcaaggaccaaatgtcaatttag
aagaaaagatagatgtggtacccattgagcctcatgctctgttcttgggaatccatggagggaagatgtgcctgtcctgtgtcaagtctggtgatgagaccagact
ccagctggaggcagttaacatcactgacctgagcgagaacagaaagcaggacaagcgcttcgccttcatccgctcagacagtggccccaccaccagttttgag
tctgccgcctgccccggttggttcctctgcacagcgatggaagctgaccagcccgtcagcctcaccaatatgcctgacgaaggcgtcatggtcaccaaattctac
ttccaggaggacgagggaagcggagagggcagaggaagtctgctaacatgcggtgacgtcgaggagaatcctggacctatggtcgatggagtgatgatcct
gcctgtcctgattatgattgccctgcccagccccagcatggaagatgaaaaacctaaagtcaaccctaagctgtatatgtgcgtgtgcgagggcctgagctgcg
gaaacgaggatcactgcgagggccagcagtgtttcagctccctgtccatcaatgacggcttccacgtgtaccagaagggctgctttcaggtgtatgagcagggc
aagatgacctgraagacaccaccttccccaggacaggcagtggagtgctgtcagggcgattggtgtaaccggaatatcaccgcccagctgccaacaaagggc
aagtctttccccggcacacagaactttcacctggaagtgggcctgatcatcctgagcgtggtgttcgccgtgtgcctgctggcatgtctgctgggagtggccctg
ggtggaggcgggtctggaggcgggggtagtggcgggggtggaagcatgggagctgctgcaaagttggcgtttgccgtctttcttatctcctgttcttcaggtgct
atacttggtagarcagaaactcaggagtgtcttttctttaatgctaattgggaaaaagacagaaccaatcaaactggtgttgaaccgtgttatggtgacaaagataaa
cggcggcattgttttgctacctggaagaatatttctggttccattgaaatagtgaaacaaggttgttggctggatgatatcaactgctatgacaggactgattgtgtag
aaaaaaaagacagccctgaagtatatttttgttgctgtgagggcaatatgtgtaatgaaaagttttcttattttccggagatggaagtcacacagcccacttcaaatcc
agttacacctaagccaccctattacaacatcctgctctattccttggtgccacttatgttaattgcggggattgtcatttgtgcattttgggCgtacaggcatcacaag
atggcctaccctcctgtacttgttccaactcaagacccaggaccacccccaccttctccattactaggtttgaaaccactgcagttattagggggggaggcgggt
cgggtggcggaggtagcggaggcggtggaatgacggcgccctgggtggccctcgccctcctctggggatcgctgtgcgccggctctgggcgtggggagg
ctgagacacgggagtgcatctactacaacgccaactgggagctggagcgcaccaaccagagcggcctggagcgctgcgaaggcgagcaggacaagcgg
ctgcactgctacgcctcctggcgcaacagctctggcaccatcgagctcgtgaagaagggctgctggctagatgacttcaactgctacgataggcaggagtgtgt
ggccactgaggagaacccccaggtgtacttctgctgctgtgaaggcaacttctgcaacgaacgcttcactcatttgccagaggctgggggcccggaagtcacg
tacgagccacccccgacagcccccaccctgctcacggtgctggcctactcactgctgcccatcgggggcctttccctcatcgtcctgctggccttttggatgtac
cggcatcgcaagcccccctacggtcatgtggacatccatgaggaccctgggcctccaccaccatcccctctggtgggcctgaagccactgcagctgctgTA
AtgtacaagtaaccTgAacaaacaccattgtcacactccaacaaacaccattgtcacactccaacaaacaccattgtcacactccatgaggatccgatctttttc
cctctgccaaaaattatggggacatcatgaagccccttgagcatctgacttctggctaataaaggaaatttattttcattgcaatagtgtgttggaattttttgtgtctctc
actcggaagcaattcgttgatctgaatttcgaccacccataatacccattaccctggtagataagtagcatggcgggttaatcattaactacaaggaacccctagtg
atggagttggccactccctctctgcgcgctcgctcgctcactgaggccgggcgaccaaaggtcgcccgacgcccgggctttgcccgggcggcctcagtgagc
gagcgagcgcgcag
>Flare-up-responsive promoter (SEQ ID NO: 21)
tacggtaaatggcccgcctggctgaccgcccaacgaccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacggtaaatggcccgcct
ggcattatgcccagtacatgaccttatgggactttcctacttggcagtacatctactcgaggccacgttctgcttcacGCTTCGCGCCCTAAGTCT
GCAGGTGACGGGCTCAGGGGCGGGGGCTGGGTGGGGGGGAGCGGAGAATGCTCCAGCCCAGTT
TGCCGTCTCCATGGCGACCGCCCGCGCGGCGCCAGCCTGACAGCCCGTCCGGGTTTTATGAATGG
GTGACGTCACGGGCCTGGCGTCTAACGGTCTGAGCCGCTTGTTCAGACGCTGACACAGACCAGC
CCGGGAAAGGTGAGCTCACAGAGGGGACTTTCCGAGAGATCTACAGAGGGGACTTTCCGAGAGC
GAGCTTGGGCTGCAGGTCGACCGTCCATCCATTCACAGCGCTTCTATAAAGGCGCCAGCTGAGG
CGCCTACTACTCCAACCGCGACTGCAGCGAGCAACTGAGAAGACTGGATAGAGCCGGCGGTTCC
GCGAACGAGCAGTGACCGCGCTCCCACCCAGCTCTGCTCTGCAGCTCCACCAGTGTCTCTCTAGA
>pAA Vss EndolD1-BRE NFKB PI-Gaussia (SEQ ID NO: 22)
cctgcaggcagctgcgcgctcgctcgctcactgaggccgcccgggcaaagcccgggcgtcgggcgacctttggtcgcccggcctcagtgagcgagcgagc
gcgcagagagggagtggccaactccatcactaggggttcctgccgcgtcgacattgattattgactctggtcgttacataacttacggtaaatggcccgcctggc
tgaccgcccaacgaccccgcccattgacgtcaataatgacgtatgttcccatagtaacgccaatagggactttccattgacgtcaatgggtggagtatttacggta
aactgcccacttggcagtacatcaagtgtatcatatgccaagtacgccccctattgacgtcaatgacggtaaatggcccgcctggcattatgcccagtacatgacc
ttatgggactttcctacttggcagtacatctactcgaggccacgttctgcttcacGCTTCGCGCCCTAAGTCTGCAGGTGACGGGCT
CAGGGGCGGGGGCTGGGTGGGGGGGAGCGGAGAATGCTCCAGCCCAGTTTGCCGTCTCCATGGC
GACCGCCCGCGCGGCGCCAGCCTGACAGCCCGTCCGGGTTTTATGAATGGGTGACGTCACGGGC
CTGGCGTCTAACGGTCTGAGCCGCTTGTTCAGACGCTGACACAGACCAGCCCGGGAAAGGTGAG
CTCACAGAGGGGACTTTCCGAGAGATCTACAGAGGGGACTTTCCGAGAGCGAGCTTGGGCTGCA
GGTCGACCGTCCATCCATTCACAGCGCTTCTATAAAGGCGCCAGCTGAGGCGCCTACTACTCCAA
CCGCGACTGCAGCGAGCAACTGAGAAGACTGGATAGAGCCGGCGGTTCCGCGAACGAGCAGTG
ACCGCGCTCCCACCCAGCTCTGCTCTGCAGCTCCACCAGTGTCTCTCTAGAgccaccgcggtggcggccctag
agtcgatcgaggaactgaaaaaccagaaagttaactggtaagtttagtctttttgtcttttatttcaggtcccggatccggtggtggtgcaaatcaaagaactgctcct
cagtggatgttgcctttacttctaggcctgtacggaagtgttacttctgctctaaaagctgcggaattgtacccgcggccgatccaccggtcgccaccatctagcat
gggagtcaaagttctgtttgccctgatctgcatcgctgtggccgaggccaagcccaccgagaacaacgaagacttcaacatcgtggccgtggccagcaacttc
gcgaccacggatctcgatgctgaccgcgggaagttgcccggcaagaagctgccgctggaggtgctcaaagagatggaagccaatgcccggaaagctggct
gcaccaggggctgtctgatctgcctgtcccacatcaagtgcacgcccaagatgaagaagttcatcccaggacgctgccacacctacgaaggcgacaaagagt
ccgcacagggcggcataggcgaggcgatcgtcgacattcctgagattcctgggttcaaggacttggagcccatggagcagttcatcgcacaggtcgatctgtg
tgtggactgcacaactggctgcctcaaagggcttgccaacgtgcagtgttctgacctgctcaagaagtggctgccgcaacgctgtgcgacctttgccagcaaga
tccagggccaggtggacaagatcaagggggccggtggtgactagctcgacgctgatcagcctcgactgtgccttctagttgccagccatctgttgtttgcccctc
ccccgtgccttccttgaccctggaaggtgccactcccactgtcctttcctaataaaatgaggaaattgcatcgcattgtctgagtaggtgtcattctattctggggggt
ggggtggggcaggacagcaagggggaggattgggaagacaaggccgcaggaacccctagtgatggagttggccactccctctctgcgcgctcgctcgctc
actgaggccgggcgaccaaaggtcgcccgacgcccgggctttgcccgggcggcctcagtgagcgagcgagcgcgcagctgcctgcagg
>pAA Vss-CB-PI(MBL-amiRACVR1-INHBA)-opt-ACVR1-miR-122T (SEQ ID NO: 23)
ctgcgcgctcgctcgctcactgaggccgcccgggcaaagcccgggcgtcgggcgacctttggtcgcccggcctcagtgagcgagcgagcgcgcagagag
ggagtggccaactccatcactaggggttccttgtagttaatgattaacccgccatgctacttatctaccagggtaatggggatcctctagaactatagctagtcgac
attgattattgactagttattaatagtaatcaattacggggtcattagttcatagcccatatatggagttccgcgttacataacttacggtaaatggcccgcctggctga
ctgcccacttggcagtacatcaagtgtatcatatgccaagtacgccccctattgacgtcaatgacggtaaatggcccgcctggcattatgcccagtacatgacctt
atgggactttcctacttggcagtacatctacgtattagtcatcgctattaccatgtcgaggccacgttctgcttcactctccccatctcccccccctccccacccccaa
gaggcggagaggtgcggcggcagccaatcagagcggcgcgctccgaaagtttccttttatggcgaggcggcggcggcggcggccctataaaaagcgaag
cgcgcggcgggcgggagcaagctttcagatcgcctggagacgccatccacgctgttttgacctccatagaagacaccgggaccgatccagcctccgcggcc
gggaacggtgcattggaacgcggattccccgtgccaagagtgacgtaagtaccgcctatagagtctataggcccacccccttggcttcttatgcatgctatactgt
ttttggcttggggtctatacacccccgcttcctcatgttTGCTGCCCGTGACCAGCACGTCAACGATTTTGTGGGCACGGG
GGTGCCTCTGGCCGGCTGCACACCTCCTGGCGGGCAGCTGTGtgtaatctggtgagccactgtTGTTCTGGCA
CGACACcgcagtgtagtctgagcagtactcgttgctgccgcgcgcgccaccagacataatagctgacagactaacagactgttcctttccatgggtcttttct
gcaAGGGCTCTGCGTTTGCTCCAGGTAGTCCGCTGCTCCCTTGGGCCTGGGCCCACTGACAGCCCT
ATACCTGACAGTGGCAGATCAGATTACACACGGAGGCCTGCCCTGACTGCCCACGGTGCCGTGG
CCAAAGAGGATCTAAGGGCACCGCTGAGGGCCTACCTAACCATCGTGGGGAATAAGGACAGTGT
CACCCtgcagtcaccgtcgccgccaccggtGGCAGCCTTGGAGTGGGTTCCTGCCCCCTCGGGCACACAAACA
GAGCTGAAGACCACCCTGGGCACCTCCTTGGCTGGCCGCATACCTCCTGGCGGGCAGCTGTGtgatc
tccgaggtctgctccaTGTTCTGGTGGTACCCATGGAGCAGTGCCCGGAGATCACACAGAGGCCTGCCTGG
CCCTCGAGAGACTGCCCTGACTGAAGGCCCTATCAGGTGGGGGAGGGGATCCTGATAGAGGGCA
CTGCTGCCACTGTTGGGGCCCAAGtgaattccgccaccatggtcgatggagtgatgatcctgcctgtcctgattatgattgccctgcccagc
cccagcatggaagatgaaaaacctaaagtcaaccctaagctgtatatgtgcgtgtgcgagggcctgagctgcggaaacgaggatcactgcgagggccagca
gtgtttcagctccctgtccatcaatgacggcttccacgtgtaccagaagggctgctttcaggtgtatgagcagggcaagatgacctgtaagacaccaccttcccca
ggacaggcagtggagtgctgtcaggggattggtgtaaccggaatatcaccgcccagctgccaacaaagggcaagtctttccccggcacacagaactttcacc
tggaagtgggætgatcatcctgagcgtggtgttcgccgtgtgcctgctggcatgtctgctgggagtggccctgagaaagtttaagcggagaaaccaggagcg
gctgaatccaagagatgtggagtacggcaccatcgagggcctgatcaccacaaatgtgggcgactctacactggccgacctgctggatcacagctgcaccag
cggctccggatctggcctgccctttctggtgcagaggaccgtggcccggcagatcaccctgctggagtgcgtgggcaagggccggtacggagaagtgtgga
gaggatcctggcagggagagaacgtggcagtgaagatcttctctagccgggatgagaagtcttggtttagagagacagagctgtataacacagtgatgctgag
gcacgagaatatcctgggcttcatcgcctccgacatgacctctcgccactcctctacacagctgtggctgatcacccactaccacgagatgggctccctgtacga
ttacctccagctgaccacactggacacagtgtcttgcctgcggatcgtgctgtctatcgccagcggcctggcacacctgcacatcgagatctttggaacccaggg
caagccagcactcgcacacagagatctgaagtctaagaacatcctggtgaagaagaatggccagtgctgtatcgccgatctgggcctggccgtgatgcacagc
cagtccaccaaccagctggacgtgggcaacaatcctcgggtgggcacaaagagatacatggccccagaggtgctggatgagacaatccaggtggactgcttc
gatagctataagagggtggacatctgggcctttggcctggtgctgtgggaggtggcaaggaggatggtgagcaacggcatcgtggaggactacaagccaccc
tagcctggatatactgaaaaccgactgctgaccTgAacaaacaccattgtcacactccaacaaacaccattgtcacactccaacaaacaccattgtcacactcc
ttctatgacgtggtgcctaatgatccatcctttgaggacatgcgcaaggtggtgtgcgtggatcagcagaggcccaacatccctaatcgctggttcagcgacccc
accctgacatccctggccaagctgatgaaggagtgttggtatcagaatcctagcgccaggctgaccgccctgcgcatcaagaaaactctgactaaaatcgacaa
atgaggatccgatctttttccctctgccaaaaattatggggacatcatgaagccccttgagcatctgacttctggctaataaaggaaatttattttcattgcaatagtgt
gttggaattttttgtgtctctcactcggaagcaattcgttgatctgaatttcgaccacccataatacccattaccctggtagataagtagcatgggggttaatcattaa
ctacaaggaacccctagtgatggagttggccactccctctctgcgcgctcgctcgctcactgaggccgggcgaccaaaggtcgcccgacgcccgggctttgc
ccgggcggcctcagtgagcgagcgagcgcgcag
>miR-122/miR-208 binding sites (SEQ ID NO: 24)
ACAAGCTTTTTGCTCGTCTTATACAAGCTTTTTGCTCGTCTTATACAAGCTTTTTGCTCGTCTTATa
caaacaccattgtcacactccaacaaacaccattgtcacactccaacaaacaccattgtcacactcca
>Wild-type human Activin A Rcceptor, type I (ACVR1) protein (SEQ ID NO: 25)
MVDGVMILPVLIMIALPSPSMEDEKPKVNPKLYMCVCEGLSCGNEDHCEGQQCFSSLSINDGFHVYQ
KGCFQVYEQGKMTCKTPPSPGQAVECCQGDWCNRNITAQLPTKGKSFPGTQNFHLEVGLIILSVVFA
VCLLACLLGVALRKFKRRNQERLNPRDVEYGTIEGLITTNVGDSTLADLLDHSCTSGSGSGLPFLVQR
TVARQITLLECVGKGRYGEVWRGSWQGENVAVKIFSSRDEKSWFRETELYNTVMLRHENILGFIASD
MTSRHSSTQLWLITHYHEMGSLYDYLQLTTLDTVSCLRIVLSIASGLAHLHIEIFGTQGKPAIAHRDLK
SKNILVKKNGQCCIADLGLAVMHSQSTNQLDVGNNPRVGTKRYMAPEVLDETIQVDCFDSYKRVDI
WAFGLVLWEVARRMVSNGIVEDYKPPFYDVVPNDPSFEDMRKVVCVDQQRPNIPNRWFSDPTLTSL
AKLMKECWYQNPSARLTALRIKKTLTKIDNSLDKLKTDC
>Human ACVR1^{R206H} protein (SEQ ID NO: 26)
MVDGVMILPVLIMIALPSPSMEDEKPKVNPKLYMCVCEGLSCGNEDHCEGQQCFSSLSINDGFHVYQ
KGCFQVYEQGKMTCKTPPSPGQAVECCQGDWCNRNITAQLPTKGKSFPGTQNFHLEVGLIILSVVFA
VCLLACLLGVALRKFKRRNQERLNPRDVEYGTIEGLITTNVGDSTLADLLDHSCTSGSGSGLPFLVQR
TVAHQITLLECVGKGRYGEVWRGSWQGENVAVKIFSSRDEKSWFRETELYNTVMLRHENILGFIASD
MTSRHSSTQLWLITHYHEMGSLYDYLQLTTLDTVSCLRIVLSIASGLAHLHIEIFGTQGKPAIAHRDLK
SKNILVKKNGQCCIADIGLAVMHSQSTNQLDVGNNPRVGTKRYMAPEVLDETIQVDCEDSYKRVDI
WAFGLVLWEVARRMVSNGIVEDYKPPFYDVVPNDPSFEDMRKVVCVDQQRPNIPNRWFSDPTLTSL
AKLMKECWYQNPSARLTALRIKKTLTKIDNSLDKLKTDC
>Human Activin A (INHBA) protein (SEQ ID NO: 27)
MPLLWLRGFLLASCWIIVRSSPTPGSEGHSAAPDCPSCALAALPKDVPNSQPEMVEAVKKHILNMLH
LKKRPDVTQPVPKAALLNAIRKLHVGKVGENGYVEIEDDIGRRAEMNELMEQTSEIITFAESGTARKT
LHFEISKEGSDLSVVERAEVWLFLKVPKANRTRTKVTIRLFQQQKHPQGSLDTGEEAEEVGLKGERSE
LLLSEKVYDARKSTWHVFPVSSSIQRLLDQGKSSLDVRIACEQCQESGASLVLLGKKKKKEEEGEGK
KKGGGEGGAGADEEKEQSHRPFLMLQARQSEDHPHRRRRRGLECDGKVNICCKKQFFVSFKDIGWN
DWIIAPSGYHANYCEGECPSHIAGTSGSSLSFHSTVINHYRMRGHSPFANLKSCCVPTKLRPMSMLYY
DDGQNIIKKDIQNMIVEECGCS
>Codon-optimized wild-type human Activin A Rcceptor. type 1 (ACVR1) (SEQ ID NO: 28)
atggtcgatggagtgatgatcctgcctgtcctgattatgattgccctgcccagccccagcatggaagatgaaaaacctaaagtcaaccctaagctgtatatgtgcg
tgtgcgagggcctgagctgcggaaacgaggatcactgcgagggccagcagtgtttcagctccctgtccatcaatgacggcttccacgtgtacccgaagggctg
ctttcaggtgtatgagcagggcaagatgacctgtaagacaccaccttccccaggacaggcagtggagtgtgtcagggcgattggtgtaaccggaatatcacc
gcccagctgccaacaaagggcaagtctttccccggcacacagaactttcacctggaagtgggcctgatcatcctgagcgtggtgttcgccgtgtgcctgctggc
atgtctgctgggagtggccctgagaaagtttaagcggagaaaccaggagcggctgaatccaagagatgtggagtacggcaccatcgagggcctgatcaccac
aaatgtgggcgactctacactggccgacctgctggatcacagctgcaccagcggctccggatctggcctgccctttctggtgcagaggaccgtggcccggca
gatcaccctgctggagtgcgtgggcaagggccggtacggagaagtgtggagaggatcctggcagggagagaacgtggcagtgaagatcttctctagccggg
atgagaagtcttggtttagagagacagagctgtataacacagtgatgctgaggcacgagaatatcctgggcttcatcgcctccgacatgacctctcgccactcctc
tacacagctgtggctgatcacccactaccacgagatgggctccctgtacgattacctccagctgaccacactggacacagtgtcttgcctgcggatcgtgctgtct
atcgccagcggcctggcacacctgcacatcgagatctttggaacccagggcaagccagcaatcgcacacagagatctgaagtctaagaacatcctggtgaag
aagaatggccagtgctgtatcgccgatctgggcctggccgtgatgcacagccagtccaccaaccagctggacgtgggcaacaatcctcggggggcacaaa
gagatacatggccccagaggtgctggatgagacaatccaggtggactgcttcgatagctataagagggtggacatctgggcctttggcctggtgctgtgggag
gtggcaaggaggatggtgagcaacggcatcgtggaggactacaagccacccttctatgacgtggtgcctaatgatccatcctttgaggacatgcgcaaggtgg
tgtgcgtggatcagcagaggcccaacatccctaatcgctggttcagcgaccccaccctgacatccctggccaagctgatgaaggagtgttggtatcagaatcct
agcgccaggctgaccgccctgcgcatcaagaaaactctgactaaaatcgacaatagcctggataacctgaaaaccgactgctga
>Human ACVR1^{R206H} mRNA (SEQ ID NO: 29) (Ts may be substituted w/ Us)
GCTCTTTGCAGCCGCCGCCGCCGCCGCAGCCTCCCCCTCGGCGCAGGGGGAACTTTTCTACTCTC
TGTGACGGTCTCCCCCGCCGCCCCCGGGGGCAAGCCCAGCTGGTCCGCCCGCCCCGCCCGCGGTC
GTGCTCCCAGCCCGAGCCTCTGCGCCTCGGGAAGTTTATTCTGCCCGCCCTCCTCCCCTTCCTCCT
CCTCTTCACCCCGCCTCCCCGCCCTCCCCCGGCTCTTTCCCCCGGGTGGCTGCCGCTGCCGCCGCT
GCCGCTGCCAGCCTGCTCCTCGGCCAGCCGCGCCGGCCCCAGGGCCGGGAAGCTCCCGGCCCCA
CGCACCGCTCCGCTGCAGCCACCGCAGCCGCCGCCGCTCTGGGCCGGCCCTCGGCCCCCGGCTCC
CGGCCCCGCAGAGTTCCGGGCTCCCTCGCCGGCTGCACCGCCCCGCCCCGCCCCGCGCCGCCCCG
CGCCGCGCCGACCTGCAGCGCCCGGCTGCCTCGCACTCCGCCTCCCCCGGCTCAGCCCCCGGCCG
CGGCGGGACCCGAGCCTGGAGCATTGGTAAGCGTCACACTGCCAAAGTGAGAGCTGCTGGAGAA
CTCATAATCCCAGGAACGCCTCTTCTACTCTTCGAGTACCCCAGTGACCAGAGTGAGAGAAGCTC
TGAACGAGGGCACGCGGCTTGAAGGACTGTGGGCAGATGTGACCAAGAGCCTGCATTAAGTTGT
ACAATGGTAGATGGAGTGATGATTCTTCCTGTGCTTATCATGATTGCTCTCCCCTCCCCTAGTATG
GAAGATGAGAAGCCCAAGGTCAACCCCAAACTCTACATGTGTGTGTGTGAAGGTCTCTCCTGCG
GTAATGAGGACCACTGTGAAGGCCAGCAGTGCTTTTCCTCACTGAGCATCAACGATGGCTTCCAC
GTCTACCAGAAAGGCTGCTTCCAGGTTTATGAGCAGGGAAAGATGACCTGTAAGACCCCGCCGT
GCCCACTAAAGGAAAATCCTTCCCTGGAACACAGAATTTCCACTTGGAGGTTGGCCTCATTATTC
CCCCTGGCCAAGCCGTGGAGTGCTGCCAAGGGGACTGGTGTAACAGGAACATCACGGCCCAGCT
TCTCTGTAGTGTTCGCAGTATGTCTTTTAGCCTGCCTGCTGGGAGTTGCTCTCCGAAAATTTAAAA
GGCGCAACCAAGAACGCCTCAATCCCCGAGACGTGGAGTATGGCACTATCGAAGGGCTCATCAC
CACCAATGTTGGAGACAGCACTTTAGCAGATTTATTGGATCATTCGTGTACATCAGGAAGTGGCT
CTGGTCTTCCTTTTCTGGTACAAAGAACAGTGGCTCGCCAGATTACACTGTTGGAGTGTGTCGGG
GCATGAAAATATCTTAGGTTTCATTGCTTCAGACATGACATCAAGACACTCCAGTACCCAGCTGT
AAAGGCAGGTATGGTGAGGTGTGGAGGGGCAGCTGGCAAGGGGAGAATGTTGCCGTGAAGATC
TTCTCCTCCCGTGATGAGAAGTCATGGTTCAGGGAAACGGAATTGTACAACACTGTGATGCTGAG
GGTTAATTACACATTATCATGAAATGGGATCGTTGTACGACTATCTTCAGCTTACTACTCTGGAT
ACAGTTAGCTGCCTTCGAATAGTGCTGTCCATAGCTAGTGGTCTTGCACATTTGCACATAGAGAT
ATTTGGGACCCAAGGGAAACCAGCCATTGCCCATCGAGATTTAAAGAGCAAAAATATTCTGGTT
AAGAAGAATGGACAGTGTTGCATAGCAGATTTGGGCCTGGCAGTCATGCATTCCCAGAGCACCA
ATCAGCTTGATGTGGGGAACAATCCCCGTGTGGGCACCAAGCGCTACATGGCCCCCGAAGTTCT
AGATGAAACCATCCAGGTGGATTGTTTCGATTCTTATAAAAGGGTCGATATTTGGGCCTTTGGAC
TTGTTTTGTGGGAAGTGGCCAGGCGGATGGTGAGCAATGGTATAGTGGAGGATTACAAGCCACC
GTTCTACGATGTGGTTCCCAATGACCCAAGTTTTGAAGATATGAGGAAGGTAGTCTGTGTGGATC
AACAAAGGCCAAACATACCCAACAGATGGTTCTCAGACCCGACATTAACCTCTCTGGCCAAGCT
AATGAAAGAATGCTGGTATCAAAATCCATCCGCAAGACTCACAGCACTGCGTATCAAAAAGACT
TTGACCAAAATTGATAATTCCCTCGACAAATTGAAAACTGACTGTTGACATTTTCATAGTGTCAA
GAAGGAAGATTTGACGTTGTTGTCATTGTCCAGCTGGGACCTAATGCTGGCCTGACTGGTTGTCA
GAATGGAATCCATCTGTCTCCCTCCCCAAATGGCTGCTTTGACAAGGCAGACGTCGTACCCAGCC
ATGTGTTGGGGAGACATCAAAACCACCCTAACCTCGCTCGATGACTGTGAACTGGGCATTTCACG
CACAGAGAAATCCTAAAAGAGATCTGGGCATTAAGTCAGTGGCTTTGCATAGCTTTCACAAGTCT
AACTGTTCACACTGCAGAGACTAATGTTGGACAGACACTGTTGCAAAGGTAGGGACTGGAGGAA
CCTAGACACTCCCCACGGGAAACTCAAGGAGGTGGTGAATTTTTAATCAGCAATATTGCCTGTGC
TTCTCTTCTTTATTGCACTAGGAATTCTTTGCATTCCTTACTTGCACTGTTACTCTTAATTTTAAAG
ACCCAACTTGCCAAAATGTTGGCTGCGTACTCCACTGGTCTGTCTTTGGATAATAGGAATTCAAT
TTGGCAAAACAAAATGTAATGTCAGACTTTGCTGCATTTTACACATGTGCTGATGTTTACAATGA
TTTTACAAAACTGCTTTGTGCATATGTTAAAGCTTATTTTTATGTGGTCTTATGATTTTATTACAG
TGCCGAACATTAGGAATTGTTTATACACAACTTTGCAAATTATTTATTACTTGTGCACTTAGTAGT
AAATGTTTTTAACACTATACTCTAAAATGGACATTTTCTTTTATTATCAGTTAAAATCACATTTTA
AGTGCTTCACATTTGTATGTGTGTAGACTGTAACTTTTTTTCAGTTCATATGCAGAACGTATTTAG
CCATTACCCACGTGACACCACCGAATATATTACTGATTTAGAAGCAAAGATTTCAGTAGAATTTT
AGTCCTGAACGCTACGGGGAAAATGCATTTTCTTCAGAATTATCCATTACGTGCATTTAAACTCT
GCCAGAAAAAAATAACTATTTTGTTTTAATCTACTTTTTGTATTTAGTAGTTATTTGTATAAATTA
AATAAACTGTTTTCAAGTCAAA
>CBA Promoter (SEQ ID NO: 30)
gcgccaggcggggggggggggcgaggggggggcggggcgaggcggagaggtgcggcggcagccaatcagagcggcgcgctccgaaagtttcct
tttatggcgaggcggcggcggcggcggccctataaaaagcgaagcgcgcggcggg
>CMV Enhancer (SEQ ID NO: 31)
ctagtcgacattgattattgactagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgacgtcaatgggtggagtatt
tacggtaaactccccacttggcagtacatcaagtgtatcatatgccaagtacgccccctattgacgtcaatgacggtaaatggcccgcctggcattatgcccagta
catgaccttatgggactttcctacttggcagtacatctacgtattagtcatcgctattac
>miR-122 Binding Site (SEQ ID NO: 32)
acaaacaccattgtcacactcca
>miR-208a Binding Site (SEQ ID NO: 33)
ACAAGCTTTTTGCTCGTCTTAT
>NF-KB Promoter (SEQ ID NO: 34)
TGAGCTCACAGAGGGGACTTTCCGAGAGATCTACAGAGGGGACTTTCCGAGAGCGAGCTTGGGC
TGCAGGTCGACCGTCCATCCATTCACAGCGCTTCTATAAAGGCGCCAGCTGAGGCGCCTACTACT
CCAACCGCGACTGCAGCGAGCAACTGAGAAGACTGGATAGAGCCGGCGGTTCCGCGAACGAGC
AGTGACCGCGCTCCCACCCAGCTCTGCTCTGCAGCTCCACCAGTGTCTCTCTAGA
>amiR-ACVR1^{R206H} (SEQ ID NO: 35)
agggctctgcgtttgctccaggtagtccgctgctcccttgggcctgggcccactgacagccctggtgcctctggccggctgcacacctcctggcgggcagctgt
gtgtaatctggtgagccactgttgttctggcaatacctgacagtggcagatcagattacacacggaggcctgccctgactgcccacggtgccgtggccaaagag
gatctaagggcaccgctgagggcctacctaaccatcgtggggaataaggacagtgtcaccc
>Human soluble Tumor Necrosis Factor Rcceptor, Type 2 (TNFR2), protein (SEQ ID NO: 36)
MAPVAVWAALAVGLELWAAAHALPAQVAFTPYAPEPGSTCRLREYYDQTAQMCCSKCSPGQHAK
VFCTKTSDTVCDSCEDSTYTQLWNWVPECLSCGSRCSSDQVETQACTREQNRICTCRPGWYCALSK
QEGCRLCAPLRKCRPGFGVARPGTETSDVVCKPCAPGTFSNTTSSTDICRPHQICNVVAIPGNASMDA
VCTSTSPTRSMAPGAVHLPQPVSTRSQHTQPTPEPSTAPSTSFLLPMGPSPPAEGSTGDFALPV
>Human soluble Interleukin-1 Rcceptor Antagonist (IL-IRα), protein (SEQ ID NO: 37)
MEICRGLRSHLITLLLFLFHSETICRPSGRKSSKMQAFRIWDVNOKTFYLRNNQLVAGYLQGPNVNLE
EKIDVVPIEPHALFLGIHGGKMCLSCVKSGDETRLQLEAVNITDLSENRKQDKRFAFIRSDSGPTTSFES
AACPGWFLCTAMEADQPVSLTNMPDEGVMVTKFYFQEDE
>pBRE nucleic acid sequence (SEQ ID NO: 38)
GCTTCGCGCCCTAAGTCTGCAGGTGACGGGCTCAGGGGCGGGGGCTGGGTGGGGGGGAGCGGA
GAATGCTCCAGCCCAGTTTGCCGTCTCCATGGCGACCGCCCGCGCGGCGCCAGCCTGACAGCCC
GTCCGGGTTTTATGAATGGGTGACGTCACGGGCCTGGCGTCTAACGGTCTGAGCCGCTTGTTCAG
ACGCTGACACAGACCAGCCCGGGAAAGG
>Human kinase-dcad ACVR2A, protein (SEQ ID NO: 39)
gctatacttggtagatcagaaactcaggagtgtcttttctttaatgctaattgggaaaaagacagaaccaatcaaactggtgttgaaccgtgttatggtgacaaagat
aaacggcggcattgttttgctacctggaagaatatttctggttccattgaaatagtgaaacaaggttgttggctggatgatatcaactgctatgacaggactgattgtg
tagaaaaaaaagacagccctgaagtatatttttgttgctgtgagggcaatatgtgtaatgaaaagttttcttatittccggagatggaagtcacacagcccacttcaaa
tccagttacacctaagccaccctattacaacatcctgctctattccttggtgccacttatgttaattgcggggattgtcatttgtgcattttgggCgtacaggcatcaca
agatggcctaccctcctgtacttgttccaactcaagacccaggaccacccccaccttctccattactaggtttgaaaccactgcagttatta
>Human kinase-dcad ACVR2B, protein (SEQ ID NO: 40)
Tctgggcgtggggaggctgagacacgggagtgcatctactacaacgccaactgggagctggagcgcaccaaccagagcggcctggagcgctgcgaagg
cgagcaggacaagcggctgcactgctacgcctcctggcgcaacagctctggcaccatcgagctcgtgaagaagggctgctggctagatgacttcaactgcta
cgataggcaggagtgtgtggccactgaggagaacccccaggtgtacttctgctgctgtgaaggcaacttctgcaacgaacgcttcactcatttgccagaggctg
ggggcccggaagtcacgtacgagccacccccgacagcccccaccctgctcacggtgctggcctattcactgctgcccatcgggggcctttccctcatcgtcct
gctggccttttggatgtaccggcatcgcaagcccccctacggtcatgtggacatccatgaggaccctgggcctccaccaccatcccctctggtgggcctgaagc
cactgcagctgctg
>human INHBA inhibitory sequence (1) (SEQ ID NO: 41) (Ts may be substituted w/ Us)
tctttcttcttcttcttgccc
>human INHBA inhibitory sequence (2) (SEQ ID NO: 42) (Ts may be substituted w/ Us)
tttgccactgtcttctctgga
>human INHBA inhibitory sequence (3) (SEQ ID NO: 43) (Ts may be substituted w/ Us)
ttcctttcaagtatctctcct
>human INHBA inhibitory sequence (4) (SEQ ID NO: 44) (Ts may be substituted w/ Us)
tgatctccgaggtctgctcca
>human INHBA inhibitory sequence (5) (SEQ ID NO: 45) (Ts may be substituted w/ Us)
ttttgatgatgttttgaccat
>amiR-ACVR 1 R2061
(SEQ ID NO: 46)
tgtaatctggtgagccactgt
*Within any one of the representative nucleic acid sequences provided herein, the thymine (T) nucelotides may be substituted with uracil (U) nucleotides (c.g., for nucleic acids in an RNA format).

What is claimed is:

1. A composition comprising:
(a) a first nucleic acid sequence encoding an inhibitory nucleic acid that targets an INHBA transcript; and
(b) a second nucleic acid sequence encoding a wild-type Activin A Receptor, type 1 (ACVR1) protein.

2. A composition comprising:
(a) a first nucleic acid sequence encoding an inhibitory nucleic acid that targets an INHBA transcript;
(b) a second nucleic acid sequence encoding a wild-type Activin A Receptor, type 1 (ACVR1) protein; and
(c) a third nucleic acid sequence encoding an inhibitory nucleic acid that targets a mutant ACVR1 transcript, optionally wherein the mutant ACVR1 transcript is a ACVR1^{R206H} transcript.

3. The composition of claim 1 or 2, wherein the inhibitory nucleic acid that targets an INHBA transcript is a double-stranded RNA (dsRNA), small interfering RNA (siRNA), short hairpin RNA (shRNA), microRNA (miRNA), or artificial microRNA (amiR) that targets an INHBA transcript.

4. The composition of any one of claims 1-3, wherein the inhibitory nucleic acid that targets an INHBA transcript comprises a nucleic acid sequence having at least 90%, at least 95%, at least 97.5%, at least 99%, or 100% identity to any one of SEQ ID NOs: 41-45.

5. The composition of claim 3 or 4, wherein the amiR that targets an INHBA transcript comprises a nucleic acid sequence having at least 90%, at least 95%, at least 97.5%, at least 99%, or 100% identity to any one of SEQ ID NOs: 3-8.

6. The composition of any one of claims 2-5, wherein the inhibitory nucleic acid that targets a mutant ACVR1 transcript is a double-stranded RNA (dsRNA), small interfering RNA (siRNA), short hairpin RNA (shRNA), microRNA (miRNA), or artificial microRNA (amiR) that targets a mutant ACVR1 transcript.

7. The composition of any one of claims 2-6, wherein the inhibitory nucleic acid that targets a mutant ACVR1 transcript comprises a nucleic acid sequence having at least 90%, at least 95%, at least 97.5%, at least 99%, or 100% identity to SEQ ID NO: 46.

8. The composition of claim 6 or 7, wherein the amiR that targets a mutant ACVR1 transcript comprises the sequence so forth in SEQ ID NO: 35.

9. The composition of any one of claims 1-8 further comprising a promoter operably linked to the first nucleic acid sequence, the second nucleic acid sequence, and/or the third nucleic acid sequence, optionally wherein the promoter is a chicken beta actin (CBA) promoter or a flare-up-responsive promoter.

10. The composition of claim 9, wherein the flare-up-responsive promoter comprises a first portion comprising a NF-κB promoter, and a second portion comprising a bone morphogenic protein (BMP) signaling-responsive promoter (pBRE).

11. The composition of any one of claim 1-10, wherein the nucleic acid sequence encoding the ACVR1 protein is codon-optimized, optionally wherein the nucleic acid sequence encoding the ACVR1 protein comprises the nucleic acid sequence of SEQ ID NO: 28.

12. The composition of any one of claims 1-11, wherein the nucleic acid sequence encoding the ACVR1 protein comprises a nucleic acid sequence having at least 90%, at least 95%, at least 97.5%, or at least 99% identity to SEQ ID NO: 30.

13. The composition of any one of claims 1-12, wherein the first nucleic acid sequence, the second nucleic acid sequence, and/or the third nucleic acid sequence further comprises one or more miRNA binding sites, optionally wherein the one or more miRNA binding sites are de-targeting miRNA binding sites.

14. The composition of claim 13, wherein the one or more miRNA binding sites comprise one or more miR-122 binding sites, one or more miR-208a binding sites, or a combination thereof,
optionally wherein the one or more miR-122 binding sites comprise or consist of the nucleic acid sequence of SEQ ID NO: 33 and/or wherein the one or more miR-208a binding sites comprise or consist of the nucleic acid sequence of SEQ ID NO: 34.

15. The composition of any one of claims 1-14, wherein the ACVR1 protein comprises an amino acid sequence having at least 90%, at least 95%, at least 97.5%, at least 99%, or 100% identity to SEQ ID NO: 25.

16. The composition of any one of claims 1-15, wherein the first nucleic acid sequence, the second nucleic acid sequence, and/or the third nucleic acid sequence are encoded within a single nucleic acid, optionally wherein the single nucleic acid further comprises one or more adeno-associated virus (AAV) inverted terminal repeats (ITRs).

17. An isolated nucleic acid comprising a transgene comprising a nucleic acid sequence encoding one or more artificial microRNAs (amiR) targeting an INHBA RNA transcript, flanked by adeno-associated virus (AAV) inverted terminal repeats (ITRs).

18. The isolated nucleic acid of claim 17, wherein the transgene further comprises a promoter operably linked to the nucleic acid sequence encoding the amiR, optionally wherein the promoter is a chicken beta actin (CBA) promoter or a flare-up-responsive promoter.

19. The isolated nucleic acid of claim 17 or 18, wherein the transgene further comprises one or more amiRs targeting ACVR1 gene, optionally wherein the one or more amiRs targeting an ACVR1^{R206H} allele, optionally wherein the one or more amiRs targeting an ACVR1^{R206H} allele comprise the sequence set forth in SEQ ID NO: 35.

20. The isolated nucleic acid of any one of claims 17-19, wherein the one or more amiRs targeting an INHBA RNA transcript comprise the sequence set forth in any one of SEQ ID NOs: 3-8.

21. The isolated nucleic acid of any one of claims 17-20, wherein the transgene further comprises:
(a) a nucleic acid sequence encoding an ACVR1 protein, a nucleic acid sequence encoding a soluble TNFR2 (sTNFR2) protein, a nucleic acid sequence encoding a soluble IL-1Rα (sIL-1Rα) protein, or a combination thereof; and/or
(b) one or more miRNA binding sites, optionally wherein the one or more miRNA binding sites comprise one or more miR-122 binding sites, one or more miR-208a binding sites, or a combination thereof.

22. An isolated nucleic acid comprising the sequence set forth in any one of SEQ ID NOs: 3-8, 21, and 24.

23. A vector comprising the composition of any one of claims 1-16 or the isolated nucleic acid of any one of claims 17-22, optionally wherein the vector is a plasmid.

24. An recombinant adeno-associated (rAAV) comprising:
(i) the composition of any one of claims 1-16 or the isolated nucleic acid of any one of claims 17-22; and
(ii) one or more AAV capsid proteins, optionally wherein the capsid protein has a tropism for muscle or bone.

25. The rAAV of claim 24, wherein the AAV capsid protein is of a serotype selected from AAV1, AAV2, AAV3, AAV4, AAV5, AAV6, AAV7, AAV8, AAV9, AAV10, or a variant thereof.

26. A pharmaceutical composition comprising:
(i) the composition of any one of claims 1-16 or the isolated nucleic acid of any one of claims 17-22, the vector of claim 23, or the rAAV of claim 24 or 25; and
(ii) a pharmaceutically acceptable excipient, optionally wherein the pharmaceutical composition is formulated for injection, optionally wherein the injection is transdermal (t.d.) injection.

27. A method for treating a disease or disorder associated with bone in a subject in need thereof, the method comprising administering the composition of any one of claims 1-16 or the isolated nucleic acid of any one of claims 17-22, the vector of claim 23, the rAAV of claim 24 or 25, or the pharmaceutical composition of claim 26 to the subject.

28. The method of claim 27, wherein the disease or disorder associated with bone is heterotopic ossification (HO) or fibrodysplasia ossificans progressiva (FOP).

29. The method of claim 27 or 28, wherein the subject is a human, optionally wherein the subject has at least one copy of a ACVR1^{R206H} allele.

30. The method of any one of claims 27-29, wherein administering comprises administration to the muscle of the subject, administration to the bone of the subject, or systemic administration.
